(19) United States
(12) Patent Application Publication
(10) Pub. No.: US 2025/0043295 A1
(43) Pub. Date: Feb. 6, 2025

(54) MODIFIED AGROBACTERIA FOR EDITING PLANTS

(71) Applicant: Tropic Biosciences UK Limited, Norwich Nrfolk (GB)

(72) Inventors: Steven RUDDER, Norwich Norfolk (GB); Eyal MAORI, Norwich Norfolk (GB); Robert Tristan GREEN, Norwich Norfolk (GB)

(73) Assignee: Tropic Biosciences UK Limited, Norwich Nrfolk (GB)

(21) Appl. No.: 18/720,593

(22) PCT Filed: Dec. 15, 2022
(86) PCT No.: PCT/EP2022/086045

§ 371 Date: Jun. 14, 2024

(30) Foreign Application Priority Data
Dec. 17, 2021 (GB) 2118416.3

(51) Int. Cl. C12N 15/82; C12N 9/22; C12N 15/11

(52) U.S. Cl. CPC C12N 15/8205; C12N 9/22; C12N 15/11; C12N 15/8274; C12N 2310/20

(57) ABSTRACT

The present invention relates to modified bacteria for introducing desired mutations in target sequences in plant cells, wherein the bacteria has reduced VirD5 activity and carries site-specific DNA editing machinery. The invention also provides methods for generating plant cells, plant parts, plants or populations thereof using such bacteria.

### CROSS-REFERENCE TO RELATED APPLICATIONS

This application is a national stage application under 35 U.S.C. § 371 of International Application No. PCT/EP2022/086045, filed internationally on Dec. 15, 2022, which claims the benefit of priority of Great Britain Application No. 2118416.3, filed on Dec. 17, 2021, the contents of each of which are hereby incorporated by reference in their entirety.

### REFERENCE TO AN ELECTRONIC SEQUENCE LISTING

The content of the electronic sequence listing (251502013400seqlist.xml; Size: 50,007 bytes; and Date of Creation: Jun. 7, 2024) is herein incorporated by reference in its entirety.

### FIELD OF THE INVENTION

The present invention relates to modified bacteria for introducing desired mutations in target sequences in plant cells and methods for generating plant cells, plant parts, plants or populations thereof using such bacteria.

### BACKGROUND OF THE INVENTION

The ability of Agrobacterium to transfer DNA to plant cells has been harnessed for the purposes of plant genetic engineering. Rhizobium and Ensifer have similar DNA transfer and genetic engineering capabilities.

Agrobacterium-mediated plant genetic transformation requires the presence of two genetic components, generally on the bacterial Ti plasmid. The first essential component is the T-DNA, a region defined by conserved 25 bp imperfect repeats called border sequences. The second genetic component is the virulence (vir) region, which is composed of at least seven major loci (virA, virB, virC, virD, virE, virF and virG) encoding components of the bacterial protein machinery mediating T-DNA processing and transfer. The VirA and VirG proteins are two-component regulators that activate the expression of other vir genes on the Ti plasmid. The remaining Vir proteins are involved in the processing, transfer and integration of the T-DNA.

A single-strand form of the T-DNA (T-strand) and Vir effector proteins are transferred to the plant cell. Following transfer, the T-strands form complexes with Vir and plant proteins that traffic through the cytoplasm and enter the nucleus. T-DNA integration into the plant genome can occur. Although transient expression of a T-DNA encoded gene can occur without integration into the genome, the integration of T-DNA into the genome establishes a permanent transformation event that permits stable expression.

An exogenous DNA sequence can be integrated into a plant genome using a binary system in which the exogenous DNA sequence is cloned into T-DNA that is encoded on a T-Binary plasmid, whereas the virulence genes are expressed from a separate plasmid (also referred to as a “helper” plasmid). Agrobacterium transformation is then used to integrate the T-DNA region including the exogenous DNA sequence into the plant genome, so that the plant expresses a new protein from the integrated exogenous DNA sequence. Accordingly, Agrobacterium is commonly used to generate transgenic plants.

The exogenous DNA sequence expressing the protein of interest may at the same time also be integrated into the plant genome via homologous recombination, without the rest of the T-DNA region, but this generally occurs at low frequency. Mutant Agrobacterium have been described, for example in WO 2015/174514, that may exhibit more efficient integration via homologous recombination of exogenous genes cloned into a T-DNA region, in particular, exogenous genes associated with seed yield or environmental stress, for example.

There is a requirement for improved methods for modifying plants.

### SUMMARY OF THE INVENTION

The inventors have developed a modified bacterium for introducing desired mutations in target sequences in plant cells and methods for generating plant cells, plant parts, plants or populations thereof using such bacteria.

The invention can be used for introducing precise mutations into a plant, with reduced or no integration of exogenous DNA into the plant genome. The invention uses a modified bacterium that has reduced VirD5 activity and that carries site-specific DNA editing machinery. The examples demonstrate that the bacterium of the invention enables gene editing to occur via transient expression of the DNA-editing agents from the T-DNA, with reduced T-DNA integration. The invention therefore allows the efficient generation of plants carrying specific mutations without integration of exogenous DNA. Such plants may be described as gene-edited, because their genome comprises specific, targeted mutations, and non-transgenic, because their genome does not comprise integrated exogenous DNA. Such plants are preferred by consumers and regulators.

A modified bacterium that has reduced VirD5 activity and that carries site-specific DNA editing machinery is particularly useful for generating gene-edited, preferably non-transgenic plants, because reducing VirD5 activity is not expected to accelerate degradation of the T-DNA molecules in the plant cell or nucleus, which would lead to reduced expression of the DNA editing machinery. Reducing VirD5 activity in accordance with the invention does not alter the stability or sequence of the T-DNA at all, allowing longer, and higher levels of transient expression. Reducing VirD5 activity also has more predictable outcomes in a range of different plants, for example compared with altering border sequences, the role of which is poorly understood. Also, reducing VirD5 activity is more compatible with expression of DNA editing machinery than, for example, altering border sequences, which could allow integration at double stranded breaks repaired by NHEJ.

Therefore, in a first aspect, the invention provides a bacterium capable of transferring nucleotide sequences to a plant cell, comprising: (a) a nucleotide sequence encoding vir genes, wherein the expression and/or activity of VirD5 is reduced or destroyed, and (b) a T-DNA sequence encoding at least one site-specific DNA-editing agent operable to introduce at least one mutation in at least one target sequence in a plant cell.

In certain embodiments, the site-specific DNA-editing agent comprises a base editor and the T-DNA sequence also encodes one or more guide RNAs specific to the target sequence or sequences. Base editors directly convert one base or base pair into another to directly generate precise point mutations in DNA. The examples demonstrate that a bacterium of the invention comprising T-DNA expressing a base editor, specifically a APOBEC base editor, and one or more guide RNAs, can effectively generate gene-edited non-transgenic plants.

In certain embodiments, the site-specific DNA-editing agent comprises a prime editor and the T-DNA sequence also encodes one or more guide RNAs that are pegRNAs specific to the target sequence or sequences. Prime editors are effective for generating targeted insertions, deletions and base conversions and generating gene-edited non-transgenic plants in accordance with the invention.

In certain embodiments, the site-specific DNA-editing agent comprises an endonuclease and the T-DNA sequence also comprises at least one donor template operable to introduce the at least one mutation via homology-dependent repair (HDR) or non-homologous end-joining (NHEJ), and optionally encodes one or more guide RNAs specific to the target sequence or sequences. HDR and NHEJ with donor templates enables precise edits to be made in the DNA and the generation of gene-edited non-transgenic plants in accordance with the invention.

The bacterium for use in the invention is capable of transferring nucleotide sequences to a plant cell. Preferred such bacteria are bacteria of the genus Agrobacterium, the genus Rhizobium, and the genus Ensifer. Procedures for mediating gene transfer using these bacteria are well characterized. In preferred embodiments, the bacterium is Agrobacterium tumefaciens. In preferred embodiments, the Agrobacterium tumefaciens bacterium is derived from Agrobacterium tumefaciens strain EHA105 or Agrobacterium tumefaciens strain AGL1. The examples demonstrate that Agrobacterium tumefaciens bacteria according to the invention are effective for generating gene-edited non-transgenic plants.

In certain embodiments, the expression and/or activity of VirD5 encoded by the nucleotide sequence encoding vir genes is destroyed. The examples demonstrate that destroying the expression and/or activity of VirD5 improves the efficiency of generating gene-edited non-transgenic plants.

In certain embodiments, the reduction or destruction of the expression and/or activity of VirD5 encoded by the nucleotide sequence encoding vir genes is mediated by at least one mutation in the sequence encoding VirD5. In preferred embodiments, the at least one mutation in the sequence encoding VirD5 is selected from the group consisting of: (a) at least one nucleotide insertion; (b) at least one nucleotide deletion; (c) an insertion-deletion (indel); (d) an inversion; (e) at least one nucleotide substitution; and (f) any combination of (a) to (e), wherein optionally the insertion or deletion is a frame shift insertion or deletion. In certain embodiments, the at least one mutation in the sequence encoding VirD5 is at least one nonsense or missense nucleotide substitution.

In certain embodiments, the at least one mutation in the sequence encoding VirD5 is an insertion and the inserted sequence encodes a selectable marker. The examples demonstrate that such a mutation is effective for disrupting the virD5 gene and additionally allows selection of the mutated sequence.

In certain embodiments, the reduction or destruction of the expression of VirD5 encoded by the nucleotide sequence encoding vir genes is mediated by expression of a silencing RNA targeting VirD5.

In preferred embodiments of the invention, the nucleotide sequence encoding vir genes is a plasmid, most preferably a Vir-helper plasmid. In certain embodiments, the plasmid is a Ti plasmid or a Ri plasmid. Vir-helper, Ti and Ri plasmids are particularly effective for mediating the delivery of genetic material to plant cells. According to some embodiments, the nucleotide sequence encoding vir genes is a plasmid which does not comprise a T-DNA sequence, such as, but not limited to, a Vir-Helper plasmid in a T-Binary system.

In certain embodiments, the at least one target sequence includes ACO or PPO, preferably ACO1 or PPO2. The examples demonstrate that the bacteria of the invention are effective for introducing mutations in ACO1 or PPO2, to obtain ACO1 or PPO2 edited non-transgenic plants.

In certain embodiments, the at least one mutation in at least one target sequence in a plant cell includes at least one mutation that results in a selectable trait in the plant cell, optionally wherein the selectable trait is herbicide resistance. Where a mutation results in a selectable trait, plants can be screened for presence of the selectable trait in order to determine which plants have the mutation. The examples demonstrate that such a process allows efficient selection of plants carrying desired edits, in particular using a bacterium 10 which introduces a mutation in a gene that results in resistance to a herbicide.

In certain embodiments, the at least one mutation in at least one target sequence in a plant cell includes at least one mutation in at least one acetolactate synthase (ALS) gene, wherein the at least one mutation in the ALS gene provides resistance to an ALS inhibitor. In preferred embodiments, the ALS gene is the acetolactate synthase 1 (ALS1) gene or the acetolactate synthase 2 (ALS2) gene in banana and wherein the plant cell is a banana cell. In preferred embodiments, the at least one mutation in the ALS gene is a substitution that introduces a substitution in the encoded amino acid sequence, preferably at Pro-187 in banana ALS1 or Pro-181 in ALS2, most preferably Pro187Ser in ALS1 or Pro181Ser in ALS2. In certain embodiments, the ALS inhibitor is a sulfonylurea, imidazolinone, triazolopyrimidine, pyrimidinyl oxybenzoate, or sulfonylamino carbonyl triazolinones, preferably wherein the ALS inhibitor is chlorsulfuron.

In certain embodiments, the T-DNA sequence encodes at least one site-specific DNA-editing agent operable to introduce at least one mutation in an additional target sequence. The examples demonstrate that the bacterium of the invention is effective for co-editing two or more target sequences at the same time. This allows for effective generation of plants carrying two or more specific mutations. Furthermore, a bacterium operable to introduce mutations in two or more target sequences enables highly efficient selection of gene-edited non-transgenic plants. This is because plants edited at the first target sequence are likely to also have been edited at the second sequence. For example, if one mutation introduces a 30 selectable trait, this can be used to select plants that are enriched for the second mutation.

Accordingly, in certain embodiments, the T-DNA sequence encodes: (a) at least one CRISPR-associated endonuclease or modified CRISPR-associated endonuclease; (b) a first guide RNA specific to a first target sequence, and (c) a second guide RNA specific to a second target sequence, wherein the at least one endonuclease is operable to introduce at least one mutation into the first target sequence, and is operable to introduce at least one mutation into the second target sequence. This co-editing approach enables the introduction of mutations at more than one target sequence. Preferably, the at least one mutation introduced into the first target sequence results in a selectable trait in the plant cell, optionally wherein the selectable trait is herbicide resistance, which allows for highly efficient selection of gene-edited non-transgenic plants carrying both mutations.

In certain embodiments, the at least one modified CRISPR-associated endonuclease is one or more base editors operable to introduce at least one mutation into the first target sequence that results in a selectable trait in the plant cell, optionally wherein the selectable trait is herbicide resistance, and operable to introduce at least one mutation into the second target sequence. The examples demonstrate that a bacterium of the invention with a T-DNA that encodes a base editor able to introduce base conversion mutations in a first and second target sequence to generate co-edited, non-transgenic plants.

In certain embodiments, the T-DNA sequence also encodes a donor template operable to introduce at least one mutation via homology-dependent repair (HDR) or non-homologous end-joining (NHEJ) into the second target sequence.

In certain embodiments, the T-DNA sequence also encodes a first donor template operable to introduce at least one mutation via homology-dependent repair (HDR) or non-homologous end-joining (NHEJ) into the first target sequence that results in a selectable trait in the plant cell, optionally wherein the selectable trait is herbicide resistance, and a second donor template operable to introduce at least one mutation into the second target sequence. In certain embodiments, the at least one modified CRISPR-associated endonuclease is one or more prime editors and the first guide RNA is a first pegRNA specific to a first target sequence and operable to introduce at least one mutation into the first target sequence that results in a selectable trait in the plant cell, optionally wherein the selectable trait is herbicide resistance, and the second guide RNA is a second pegRNA specific to a second target sequence and operable to introduce at least one mutation into the second target sequence.

In certain embodiments, the at least one CRISPR-associated endonuclease or modified CRISPR-associated endonuclease comprise at least two different endonucleases, wherein a first endonuclease is operable to introduce at least one mutation into the first target sequence, and a second endonuclease is operable to introduce at least one mutation into the second target sequence. By using different endonucleases, more powerful and efficient gene editing can be achieved. In certain such embodiments, the first endonuclease is not operable to introduce any mutation into the second target sequence and/or the second endonuclease is not operable to introduce any mutation into the first target sequence. Such an arrangement allows different editing actions to be performed at the first and second target sequences, without risk of undesired editing outcomes occurring. In such embodiments, the first endonuclease may form a ribonucleoprotein complex with a first guide RNA specific for a first target sequence, and the second endonuclease may form a ribonucleoprotein complex with a second guide RNA specific for a second target sequence. For example, an endonuclease may be used that incorporates a specific type of guide RNA. For example, Cas9 uses both crRNA and trRNA to form an sgRNA, whilst Cas12a only requires a crRNA. Therefore, in certain embodiments, the first endonuclease is Cas9 or a modified Cas9 and the first guide RNA is a sgRNA, and the second endonuclease is Cas12a or a modified Cas12a and the second guide RNA is a crRNA, or the first endonuclease is Cas12a or a modified Cas12a and the first guide RNA is a crRNA, and the second endonuclease is Cas9 or a modified Cas9 and the second guide RNA is a sgRNA. Alternatively, or in combination, the first and second endonucleases may recognise different PAM sequences. In such embodiments the first and second endonucleases may be separately selected from SpCas9, CjCas9 and Cas12a.

In certain embodiments, the first and the second endonuclease are both operable to introduce at least one mutation into the first target sequence and/or are both operable to introduce at least one mutation into the second target sequence. In such embodiments, the first endonuclease and/or the second endonuclease may form complexes with both a first guide RNA specific for a first target sequence and a second guide RNA specific for a second target sequence. For example, the first and second endonucleases may recognise the same PAM sequence and/or incorporate the same type of guide RNA. Such an arrangement will generate greater editing diversity. When different endonucleases are used, they may preferably introduce different modifications. For example, the first endonuclease may be a base editor and the second endonuclease a prime editor, or vice versa. Or the first endonuclease may be a base editor and the second endonuclease a CRISPR-associated endonuclease such as Cas9, or vice versa. Accordingly, the T-DNA may also encode a donor template operable to introduce at least one mutation via homology-dependent repair (HDR) or non-homologous end-joining (NHEJ) into the first or second target sequence. In any of the above embodiments, the at least one mutation introduced into the first target sequence results in a selectable trait in the plant cell, optionally wherein the selectable trait is herbicide resistance, which allows for highly efficient selection of gene-edited non-transgenic plants carrying both mutations.

In certain embodiments, the nucleotide sequence encoding vir genes, optionally a Vir-helper plasmid, a Ti plasmid or Ri plasmid, does not include a T-DNA cassette and the T-DNA is encoded by an additional plasmid. The examples demonstrate that a bacterium with a Vir-helper plasmid which comprises the vir gene operon, and a second binary plasmid which encodes the T-DNA, is effective for generating gene-edited non-transgenic plants.

In certain embodiments, the additional plasmid encodes one or more additional elements selected from the group consisting of: a plant selectable marker, a bacterial selectable marker, a reporter gene, and at least one bacterial origin of replication.

In certain embodiments, the plant cell is selected from the group consisting of: cell of a suspension culture (such as an Embryonic Cell Suspension), embryogenic cell, cell of a meristematic region, cell of a callus tissue, leave cell, root cell, shoot cell, somatic cell, flower cell, pollen cell, microspore, protoplast, and a combination thereof.

In certain embodiments, the plant cell is of banana, coffee, or rice.

In certain embodiments, the banana is selected from the group consisting of: Musa acuminata, Musa balbisiana, Musa itinerans, autotriploid Musa acuminata ‘Cavendish’, and autotriploid Musa acuminata ‘Gros Michel’. The examples demonstrate that the bacteria and methods of the invention are effective for generating gene-edited non-transgenic banana, particularly Cavendish banana.

In a second aspect, the invention provides a method of generating a plant, plant part, plant cell or population thereof comprising at least one mutation in at least one target sequence, the method comprising contacting a plant, plant part or plant cell with the bacterium of the invention, optionally wherein the method further comprises regenerating said cell or plant part to obtain a whole plant. The examples demonstrate that the method of the invention effectively and efficiently generates plants, plant parts, plant cells or populations thereof that contains the desired mutation in a target sequence but are non-transgenic.

In certain embodiments, the at least one mutation in at least one target sequence includes: (a) at least one mutation in a first target sequence which results in a selectable trait in the plant, plant part or plant cell, optionally wherein the selectable trait is herbicide resistance, optionally wherein the first target sequence is an ALS gene and optionally wherein the mutation provides herbicide resistance to ALS inhibitors; and (b) at least one mutation in a second gene. The examples demonstrate that introducing a mutation that results in a selectable trait and a mutation in a second gene enables highly efficient selection of gene-edited non-transgenic plants. This is because plants edited at the first target sequence and carrying the selectable trait are likely to also have been edited at the second sequence. The selectable trait can be used to select plants that are enriched for the second mutation, as shown in the examples. The examples demonstrate that introducing mutations in ALS genes is effective for providing resistance to an ALS inhibitor, which allows effective selection.

In certain embodiments, at least one of the mutations in a target sequence results confers a selectable trait to the plant cell, plant part or plant; optionally wherein the selectable trait is herbicide resistance; optionally wherein at least one of the target sequences is in an ALS gene and wherein the mutation provides herbicide resistance to ALS inhibitors.

In preferred embodiments, the genome of the plant, plant part or plant cell that is generated does not comprise any integrated T-DNA sequence. The examples demonstrate that the method of the invention can be used to produce plants which do have any integrated T-DNA sequence, with high frequency. A lack of T-DNA sequence integration is attractive to growers and consumers and presents greatly reduced potential issues for growers wishing to obtain certain types of regulatory approval.

In certain embodiments, the method further comprises selecting at least one plant cell, plant part or plant that comprises at least one mutation in the target sequence or sequences and does not comprise any integrated T-DNA sequence in its genome. In certain embodiments, said selection comprises genotyping.

In certain embodiments, the method further comprises selecting a cell, plant part or plant having the selectable trait.

In preferred embodiments, the selectable trait is herbicide resistance and selecting is by selecting cells, plant parts or plants which are herbicide resistant; optionally wherein the target sequence is an ALS gene and selecting is by selecting cells, plant parts or plants which are resistant to ALS inhibitors.

In certain embodiments, at least 40%, optionally at least 55%, preferably at least 65% of the plant cells, plant parts or plants in the population resulting from the method do not comprise integrated T-DNA in their genome and comprise a mutation in the second gene. The examples demonstrate that the methods of the invention are effective for providing populations highly enriched in plants comprising a mutation in a second gene and which do not have any integrated T-DNA sequence, when the mutation in the first sequence has been selected for.

In certain embodiments, the method comprises: (a) introducing at least one mutation in a first target sequence that results in a selectable trait in the plant cell, optionally wherein the selectable trait is herbicide resistance, (b) introducing at least one mutation into a second target sequence, (c) selecting a plant, plant part, plant cell or population thereof that comprises the selectable trait, optionally by treating with a herbicide, wherein the selected plant, plant part, plant cell or population comprises or is enriched for the mutation in the second target sequence, and optionally wherein the selected plant, plant part, plant cell or population does not comprise integrated T-DNA in their genome, or is enriched for plants, plant parts or plant cells that do not comprise integrated T-DNA in their genome.

In certain embodiments, the method further comprises generating at least one plant embryo, plant part or plant from the cell, plant part or plant selected.

In certain embodiments, said plant, plant part or plant cell is banana, coffee, or rice.

In preferred embodiments, said plant, plant part or plant cell is of a banana cultivar selected from the group consisting of Musa acuminata, Musa balbisiana, Musa itinerans, autotriploid Musa acuminata ‘Cavendish’, and autotriploid Musa acuminata ‘Gros Michel’.

In a third aspect, the invention provides a plant, plant part, plant cell or population thereof generated by the method of the invention.

In certain embodiments, at least 40%, optionally at least 55%, preferably at least 65% of the plant cells, plant parts or plants in the population resulting from the method do not comprise integrated T-DNA in their genome and comprise a mutation in the second gene. The examples demonstrate that plant populations resulting from the methods of the invention are highly enriched for plants comprising a mutation in a second gene and which do not have any integrated T-DNA sequence, when the mutation in the first sequence has been selected for.

In preferred embodiments, the invention provides an Agrobacterium cell comprising: (a) a Vir-helper plasmid encoding vir genes, wherein the expression and/or activity of VirD5 is destroyed, and (b) a T-DNA sequence encoding at least one base editor operable to introduce at least one mutation in at least one target sequence in a plant cell, and one or more guide RNAs specific to the at least one target sequence in a plant cell. In particularly preferred embodiments, the base editor is a fusion polypeptide comprising a modified CRISPR-associated endonuclease and a deaminase moiety, preferably APOBEC.

In preferred embodiments, the invention provides an Agrobacterium cell comprising: (a) a Vir-helper plasmid encoding vir genes, wherein the expression and/or activity of VirD5 is destroyed, and (b) a T-DNA sequence encoding at least one prime editor operable to introduce at least one mutation in at least one target sequence in a plant cell, and one or more guide RNAs that are pegRNAs specific to the at least one target sequence in a plant cell. In particularly preferred embodiments, the prime editor is a fusion polypeptide comprising nCas9 and a reverse transcriptase.

In preferred embodiments, the invention provides a bacterium cell capable of transferring nucleotide sequences to a plant cell, preferably an Agrobacterium cell, comprising: (a) a Vir-helper encoding vir genes, wherein the expression and/or activity of VirD5 is destroyed, and (b) a T-DNA sequence encoding at least one CRISPR-associated endonuclease or modified CRISPR-associated endonuclease operable to introduce at least one mutation in at least one target sequence in a plant cell and one or more guide RNAs specific to the at least one target sequence in a plant cell, wherein the at least one mutation in at least one target sequence comprises a mutation which results in a selectable trait in the plant cell, optionally wherein the selectable trait is herbicide resistance, optionally wherein the at least one mutation includes at least one mutation in at least one acetolactate synthase (ALS) gene, wherein the at least one mutation in the ALS gene provides resistance to an ALS inhibitor.

In preferred embodiments, the invention provides an Agrobacterium cell comprising: (a) a Vir-helper plasmid encoding vir genes, wherein the expression and/or activity of VirD5 is destroyed, and (b) a T-DNA sequence encoding at least one CRISPR-associated endonuclease or modified CRISPR-associated endonuclease operable to introduce at least one mutation in at least one target sequence in a plant cell, one or more guide RNAs specific to the at least one target sequence in a plant cell and at least one donor template operable to introduce the at least one mutation via homology-dependent repair (HDR).

In preferred embodiments, the invention provides an Agrobacterium cell comprising: (a) a Vir-helper plasmid encoding vir genes, wherein the expression and/or activity of VirD5 is destroyed, and (b) a T-DNA sequence encoding at least one CRISPR-associated endonuclease or modified CRISPR-associated endonuclease operable to introduce at least one mutation in at least one target sequence in a plant cell, one or more guide RNAs specific to the at least one target sequence in a plant cell and at least one donor template operable to introduce the at least one mutation via homology-dependent repair (HDR) or Non-Homologous End Joining (NHEJ).

In some embodiments, the invention provides an Agrobacterium cell comprising:
(a) a Vir-helper plasmid encoding vir genes, wherein the expression and/or activity of VirD5 is reduced or destroyed, and
(b) a T-DNA sequence encoding:
   i) at least one CRISPR-associated endonuclease or modified CRISPR-associated endonuclease,
   ii) a first guide RNA specific to a first target sequence,
   iii) a second guide RNA specific to a second target sequence, and
   iv) a donor template operable to introduce the at least one mutation via homology-dependent repair (HDR) or Non-Homologous End Joining (NHEJ) into the second target sequence,
wherein the at least one CRISPR-associated endonuclease or modified CRISPR-associated endonuclease is operable to introduce at least one mutation into the first target sequence and is operable to introduce at least one mutation into the second target sequence via said donor template; optionally wherein the at least one mutation in the first target sequence results in a selectable trait in a plant cell, optionally wherein the selectable trait is herbicide resistance, optionally wherein the at least one mutation includes at least one mutation in at least one acetolactate synthase (ALS) gene, wherein the at least one mutation in the ALS gene provides resistance to an ALS inhibitor.

In some embodiments, the invention provides an Agrobacterium cell comprising:
(a) a Vir-helper plasmid encoding vir genes, wherein the expression and/or activity of VirD5 is reduced or destroyed, and
(b) a T-DNA sequence encoding:
   i) at least one CRISPR-associated endonuclease or modified CRISPR-associated endonuclease,
   ii) a first guide RNA specific to a first target sequence,
   iii) a second guide RNA specific to a second target sequence,
   iv) a first donor template operable to introduce the at least one mutation via homology-dependent repair (HDR) or Non-Homologous End Joining (NHEJ) into the first target sequence, and
   v) a second donor template operable to introduce the at least one mutation via homology-dependent repair (HDR) or Non-Homologous End Joining (NHEJ) into the second target sequence;
wherein the at least one CRISPR-associated endonuclease or modified CRISPR-associated endonuclease is operable to introduce at least one mutation into the first target sequence via said second donor template and is operable to introduce at least one mutation into the second target sequence via said second donor template; optionally wherein the at least one mutation in the first target sequence results in a selectable trait in a plant cell, optionally wherein the selectable trait is herbicide resistance, optionally wherein the at least one mutation includes at least one mutation in at least one acetolactate synthase (ALS) gene, wherein the at least one mutation in the ALS gene provides resistance to an ALS inhibitor.

In preferred embodiments, the invention provides an Agrobacterium cell comprising:
(a) a Vir-helper plasmid encoding vir genes, and (b) a T-DNA sequence encoding:
   i) at least one base editor,
   ii) a first guide RNA specific to at least one ALS gene, and
   iii) a second guide RNA specific to a second target sequence,
   wherein the at least one base editor is operable to introduce at least one mutation into the at least one ALS gene that provides resistance to an ALS inhibitor, and is operable to introduce at least one mutation into the second target sequence. In particularly preferred embodiments, the base editor is a fusion polypeptide comprising a modified CRISPR-associated endonuclease and a deaminase moiety, preferably APOBEC.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding:
i) at least one base editor,
ii) a first guide RNA specific to at least one ALS gene,
iii) a second guide RNA specific to a second target sequence, and
iv) a first donor template operable to introduce at least one mutation into the ALS gene via homology-dependent repair (HDR) or Non-Homologous End Joining (NHEJ),
wherein the at least one base editor is operable to introduce the at least one mutation into the at least one ALS gene that provides resistance to an ALS inhibitor, and is operable to introduce at least one mutation into the second target sequence. In particularly preferred embodiments, the base editor is a fusion polypeptide comprising a modified CRISPR-associated endonuclease and a deaminase moiety, preferably APOBEC.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding:
i) at least one prime editor,
ii) a first guide RNA that is a pegRNA and is specific to at least one ALS gene, and
iii) a second guide RNA that is a pegRNA and is specific to a second target sequence,
wherein the at least one prime editor is operable to introduce at least one mutation into the at least one ALS gene that provides resistance to an ALS inhibitor, and is operable to introduce at least one mutation into the second target sequence. In particularly preferred embodiments, the prime editor is a fusion polypeptide comprising nCas9 and a reverse transcriptase.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding:
i) at least one CRISPR-associated endonuclease or modified CRISPR-associated endonuclease,
ii) a first guide RNA specific to at least one ALS gene,
iii) a second guide RNA specific to a second target sequence,
iii) a first donor template, and
iv) a second donor template,
wherein the at least one CRISPR-associated endonuclease or modified CRISPR-associated endonuclease and the first donor template are operable to introduce at least one mutation via HDR or NHEJ into the at least one ALS gene that provides resistance to an ALS inhibitor, and the at least one CRISPR-associated endonuclease or modified CRISPR-associated endonuclease and the second donor template are operable to introduce at least one mutation via HDR or NHEJ into the second target sequence.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding at least one base editor operable to introduce at least one mutation in at least one target sequence in a banana cell, and one or more guide RNAs specific to the at least one target sequence in a banana cell, optionally wherein the at least one target sequence comprises an ALS gene. In particularly preferred embodiments, the base editor is a fusion polypeptide comprising a modified CRISPR-associated endonuclease and a deaminase moiety, preferably APOBEC.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding at least one prime editor operable to introduce at least one mutation in at least one target sequence in a banana cell, and one or more guide RNAs that are pegRNAs specific to the at least one target sequence in a banana cell, optionally wherein the at least one target sequence comprises an ALS gene. In particularly preferred embodiments, the prime editor is a fusion polypeptide comprising nCas9 and a reverse transcriptase.

In preferred embodiments, the invention provides an Agrobacterium cell comprising: (a) a Vir-helper plasmid encoding vir genes, wherein the expression and/or activity of VirD5 is reduced or destroyed, and (b) a T-DNA sequence encoding at least one CRISPR-associated endonuclease or modified CRISPR-associated endonuclease operable to introduce at least one mutation in at least one target sequence in a banana cell, one or more guide RNAs specific to the at least one target sequence in a banana cell and at least one donor template operable to introduce the at least one mutation via homology-dependent repair (HDR).

In preferred embodiments, the invention provides an Agrobacterium cell comprising: (a) a Vir-helper plasmid encoding vir genes, wherein the expression and/or activity of VirD5 is destroyed, and (b) a T-DNA sequence encoding:
i) at least one base editor,
ii) a first guide RNA specific to at least one ALS gene in a banana cell, and
iii) a second guide RNA specific to a second target sequence in a banana cell,
wherein the at least one base editor is operable to introduce at least one mutation into the at least one ALS gene that provides resistance to an ALS inhibitor, and is operable to introduce at least one mutation into the second target sequence. In particularly preferred embodiments, the base editor is a fusion polypeptide comprising a modified CRISPR-associated endonuclease and a deaminase moiety, preferably APOBEC.

Accordingly, the invention provides the following numbered embodiments:
1. A bacterium capable of transferring nucleotide sequences to a plant cell, comprising:
   (a) a nucleotide sequence encoding vir genes, wherein the expression and/or activity of VirD5 is reduced or destroyed, and
   (b) a T-DNA sequence encoding at least one site-specific DNA-editing agent operable to introduce at least one mutation in at least one target sequence in a plant cell.
2. The bacterium of embodiment 1, wherein the site-specific DNA editing agent comprises an endonuclease selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease.
3. The bacterium of embodiment 1, wherein the site-specific DNA-editing agent comprises a CRISPR-associated endonuclease or a modified CRISPR-associated endonuclease and the T-DNA sequence also encodes one or more guide RNAs specific to the at least one target sequence in a plant cell.
4. The bacterium of embodiment 1, wherein the site-specific DNA-editing agent comprises a CRISPR-associated endonuclease or a modified CRISPR-associated endonuclease selected from the group consisting of: a base editor, a prime editor, a Cas9 endonuclease, or an endonuclease selected from the group consisting of SpCas9, xCas9, SpCas9-NG, SaCas9, AsCpf1, LbCpf1, CjCas9, NmCas9, StCas9, TdCas9, eSpCas9, HypaCas9, Cas9-SpRY/SpG, Cas4-Cas1-Cas2 complex and MAD7.
5. The bacterium of embodiment 1, wherein the site-specific DNA-editing agent comprises a base editor and the T-DNA sequence also encodes one or more guide RNAs specific to the at least one target sequence in a plant cell.
6. The bacterium of embodiment 5, wherein the base editor is a fusion polypeptide comprising a modified CRISPR-associated endonuclease and a deaminase moiety.
7. The bacterium of embodiment 6, wherein the modified CRISPR-associated endonuclease is nCas9 or dCas9 and the deaminase moiety is a cytidine deaminase moiety or an adenine deaminase moiety.
8. The bacterium of embodiment 6, wherein the base editor is selected from the group consisting of: APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VQR-ABE, VRER-ABE, and SaKKH-ABE.
9. The bacterium of embodiment 1, wherein the site-specific DNA-editing agent comprises a prime editor and the T-DNA sequence also encodes one or more guide RNAs that are pegRNAs specific to the at least one target sequence in a plant cell.
10. The bacterium of embodiment 9, wherein the prime editor is a fusion polypeptide comprising a modified CRISPR-associated endonuclease and a reverse transcriptase.
11. The bacterium of embodiment 10, wherein the modified CRISPR-associated endonuclease is nCas9.
12. The bacterium of embodiment 10, wherein the prime editor is selected from the group consisting of PE2, PE2-VQR, PE2-VRQR, PE2-VRER, PE2-NG, PE2-SpG, PE2-SpRY, PE2*, PE4, PE5 and PE3.
13. The bacterium of embodiment 1, wherein the site-specific DNA-editing agent comprises an endonuclease, such as a CRISPR-associated endonuclease, a modified CRISPR-associated endonuclease, a transcription activator-like effector nuclease or a zinc finger nuclease, and the T-DNA sequence also encodes at least one donor template operable to introduce the at least one mutation via homology-dependent repair (HDR) or non-homologous end-joining (NHEJ), and optionally encodes one or more guide RNAs specific to the at least one target sequence in a plant cell.
14. The bacterium of any preceding embodiment, wherein the bacterium is of the genus Agrobacterium, the genus Rhizobium, or the genus Ensifer, optionally wherein the bacterium is selected from the group consisting of: Agrobacterium tumefaciens, Agrobacterium fabrum str. C58, Agrobacterium genomosp, Agrobacterium sp. S2/73, Agrobacterium sp. 13-2099-1-2, Agrobacterium sp. NCPPB 925, Agrobacterium rhizogenes, Agrobacterium salinitolerans, Agrobacterium vitis, Agrobacterium arsenijevicii, Agrobacterium deltaense, Agrobacterium larrymoorei, Rhizobium sp. AB2/73, Rhizobium sp. 16-488-2b, Rhizobium sp. 16-488-2a, Rhizobium sp. 16-449-1b, Rhizobium sp. L58/93, Rhizobium sp. L245/93, Rhizobium sp. E27B/91, Rhizobium sp. K1/93, Rhizobium sp. BK007, Rhizobium tumorigenes, Rhizobium skierniewicense, Rhizobium lusitanum, Neorhizobium sp. NCHU2750, Neorhizobium galegae, Ensifer sp. YR511, and Ensifer adhaerens.
15. The bacterium of embodiment 14, wherein the bacterium is Agrobacterium tumefaciens.
16. The bacterium of embodiment 15, wherein the Agrobacterium tumefaciens bacterium is derived from Agrobacterium tumefaciens strain EHA105 or Agrobacterium tumefaciens strain AGL1.
17. The bacterium of any preceding embodiment, wherein the expression and/or activity of VirD5 encoded by the nucleotide sequence encoding vir genes is destroyed.
18. The bacterium of any preceding embodiment, wherein said reduction or destruction of the expression and/or activity of VirD5 encoded by the nucleotide sequence encoding vir genesis mediated by at least one mutation in the sequence encoding VirD5.
19. The bacterium of embodiment 18, wherein said at least one mutation in the sequence encoding VirD5 is selected from the group consisting of:
(a) at least one nucleotide insertion;
(b) at least one nucleotide deletion;
(c) an insertion-deletion (indel);
(d) an inversion;
(e) at least one nucleotide substitution; and
(f) any combination of (a) to (e);
   wherein optionally the insertion or deletion is a frame shift insertion or deletion.
20. The bacterium of embodiment 19, wherein the said at least one mutation in the sequence encoding VirD5 is at least one nonsense or missense nucleotide substitution.
21. The bacterium of embodiment 19, wherein said at least one mutation in the sequence encoding VirD5 is an insertion and the inserted sequence encodes a selectable marker.
22. The bacterium of any preceding embodiment, wherein said reduction or destruction of the expression of VirD5 encoded by the nucleotide sequence encoding vir genes is mediated by expression of a silencing RNA targeting VirD5.
23. The bacterium of any preceding embodiment, wherein the nucleotide sequence encoding vir genes is a plasmid, such as Vir-helper plasmid, a Ti plasmid or a Ri plasmid.
24. The bacterium of any preceding embodiment, wherein the plant cell is a banana cell and at least one target sequence includes ACO or PPO, preferably ACO1 or PPO2.
25. The bacterium of any preceding embodiment, wherein the at least one mutation in at least one target sequence in a plant cell includes at least one mutation that results in a selectable trait in the plant cell, optionally wherein the selectable trait is herbicide resistance.
26. The bacterium of any preceding embodiment, wherein the at least one mutation in at least one target sequence in a plant cell includes at least one mutation in at least one acetolactate synthase (ALS) gene, wherein the at least one mutation in the ALS gene provides resistance to an ALS inhibitor.
27. The bacterium of embodiment 26, wherein the ALS gene is the acetolactate synthase 1 (ALS1) gene or the acetolactate synthase 2 (ALS2) gene in banana and wherein the plant cell is a banana cell.
28. The bacterium of embodiment 26 or 27 wherein the at least one mutation in the ALS gene is a substitution that introduces a substitution in the encoded amino acid sequence, preferably at Pro-187 in banana ALS1 or Pro-181 in ALS2, most preferably Pro187Ser in ALS1 or Pro181Ser in ALS2.
29. The bacterium of any one of embodiments 26-28, wherein the ALS inhibitor is a sulfonylurea, imidazolinone, triazolopyrimidine, pyrimidinyl oxybenzoate, or sulfonylamino carbonyl triazolinones, preferably wherein the ALS inhibitor is chlorsulfuron.
30. The bacterium of any one of embodiments 25-29, wherein the T-DNA sequence encodes at least one site-specific DNA-editing agent operable to introduce at least one mutation in an additional target sequence.
31. The bacterium of any of the preceding embodiments, wherein the T-DNA sequence encodes:
a) at least one CRISPR-associated endonuclease or modified CRISPR-associated endonuclease,
b) a first guide RNA specific to a first target sequence, and
c) a second guide RNA specific to a second target sequence, wherein the at least one endonuclease is operable to introduce at least one mutation into the first target sequence and is operable to introduce at least one mutation into the second target sequence.
32. The bacterium of embodiment 31, wherein the at least one modified CRISPR-associated endonuclease is one or more base editors operable to introduce at least one mutation into the first target sequence and operable to introduce at least one mutation into the second target sequence.
33. The bacterium of embodiment 31, wherein (a) the T-DNA sequence also encodes a donor template operable to introduce at least one mutation via homology-dependent repair (HDR) or non-homologous end-joining (NHEJ) into the second target sequence; or (b) the T-DNA sequence also encodes a first donor template operable to introduce at least one mutation via homology-dependent repair (HDR) or non-homologous end-joining (NHEJ) into the first target sequence and a second donor template operable to introduce at least one mutation via homology-dependent repair (HDR) or non-homologous end-joining (NHEJ) into the second target sequence.
34. The bacterium of embodiment 31, wherein the at least one modified CRISPR-associated endonuclease is one or more prime editors and the first guide RNA is a first pegRNA specific to a first target sequence and operable to introduce at least one mutation into the first target sequence and the second guide RNA is a second pegRNA specific to a second target sequence and operable to introduce at least one mutation into the second target sequence.
35. The bacterium of embodiment 31, wherein the at least one CRISPR-associated endonuclease or modified CRISPR-associated endonuclease comprise at least two different endonucleases, and wherein a first endonuclease is operable to introduce at least one mutation into the first target sequence, and a second endonuclease is operable to introduce at least one mutation into the second target sequence.
36. The bacterium of embodiment 35, wherein the first endonuclease is not operable to introduce any mutation into the second target sequence and/or the second endonuclease is not operable to introduce any mutation into the first target sequence.
37. The bacterium of embodiment 36, wherein the first and second endonucleases recognise different PAM sequences.
38. The bacterium of embodiment 35, wherein the first and the second endonuclease are both operable to introduce at least one mutation into the first target sequence and/or are both operable to introduce at least one mutation into the second target sequence.
39. The bacterium of embodiment 38, wherein the first and second endonucleases recognise the same PAM sequence.
40. The bacterium of embodiment 38 or 39, wherein:
(i) the first endonuclease is a base editor and the second endonuclease is a prime editor and the second guide RNA is a pegRNA, or wherein the first endonuclease is a prime editor and the first guide RNA is a pegRNA and the second endonuclease is a base editor; or
(ii) the first endonuclease is a base editor and the second endonuclease is a CRISPR-associated endonuclease such as Cas9, or wherein the second endonuclease is a CRISPR-associated endonuclease such as Cas9 and the first endonuclease is a base editor.
41. The bacterium of any of embodiments 31-40, wherein the at least one mutation introduced into the first target sequence results in a selectable trait in the plant cell, optionally wherein the selectable trait is herbicide resistance, optionally wherein the at least one mutation includes at least one mutation in at least one acetolactate synthase (ALS) gene, wherein the at least one mutation in the ALS gene provides resistance to an ALS inhibitor.
42. The bacterium of any preceding embodiment, wherein the nucleotide sequence encoding vir genes does not include a T-DNA sequence and wherein the T-DNA is encoded by an additional plasmid.
43. The bacterium of embodiment 42, wherein the additional plasmid encodes one or more additional elements selected from the group consisting of: a plant selectable marker, a bacterial selectable marker, a reporter gene, and at least one bacterial origin of replication.
44. The bacterium of any preceding embodiment, wherein the plant cell is selected from the group consisting of: cell of a suspension culture (such as an Embryonic Cell Suspension), embryogenic cell, cell of a meristematic region, cell of a callus tissue, leave cell, root cell, shoot cell, somatic cell, flower cell, pollen cell, microspore, protoplast, and a combination thereof.
45. The bacterium of any preceding embodiment, wherein the plant cell is of banana, coffee, or rice.
46. The bacterium of embodiment 45, wherein the banana is selected from the group consisting of: Musa acuminata, Musa balbisiana, Musa itinerans, autotriploid Musa acuminata ‘Cavendish’, and autotriploid Musa acuminata ‘Gros Michel’.
47. A method of generating a plant, plant part, plant cell or population thereof comprising at least one mutation in at least one target sequence, the method comprising contacting a plant, plant part or plant cell with the bacterium of any one of the preceding embodiments, optionally wherein the method further comprises regenerating said cell or plant part to obtain a whole plant.
48. The method of embodiment 47, wherein the at least one mutation in at least one target sequence includes:
a. at least one mutation in a first target sequence which results in a selectable trait in the plant, plant part or plant cell, optionally wherein the selectable trait is herbicide resistance, optionally wherein the first target sequence is an ALS gene and optionally wherein the mutation provides herbicide resistance to ALS inhibitors; and
b. at least one mutation in a second gene.
49. The method of embodiment 47, wherein at least one of the mutations in a target sequence confers a selectable trait to the plant cell, plant part or plant; optionally wherein the selectable trait is herbicide resistance; optionally wherein at least one of the target sequences is in an ALS gene and wherein the mutation provides herbicide resistance to ALS inhibitors.
50. The method of any one of embodiments 47-49, wherein the genome of the plant, plant part or plant cell that is generated does not comprise any integrated T-DNA sequence.
51. The method of any one of embodiments 47-50, further comprising selecting at least one plant cell, plant part or plant that comprises at least one mutation in the target sequence or sequences and does not comprise any integrated T-DNA sequence in its genome.
52. The method of embodiment 51, wherein said selection comprises genotyping.
53. The method of any one of embodiments 48-52, wherein the method further comprises selecting a cell, plant part or plant having the selectable trait.
54. The method of embodiment 53, wherein the selectable trait is herbicide resistance and selecting is by selecting cells, plant parts or plants which are herbicide resistant; optionally wherein the target sequence is an ALS gene and selecting is by selecting cells, plant parts or plants which are resistant to ALS inhibitors.
55. The method of any one of embodiments 48-54, wherein at least 40%, optionally at least 55%, preferably at least 65% of the plant cells, plant parts or plants in the population resulting from the method do not comprise integrated T-DNA in their genome and comprise a mutation in the second gene.
56. The method of embodiment 47, wherein the method comprises:
a) introducing at least one mutation in a first target sequence that results in a selectable trait in the plant cell, optionally wherein the selectable trait is herbicide resistance,
b) introducing at least one mutation into a second target sequence,
c) selecting a plant, plant part, plant cell or population thereof that comprises the selectable trait, optionally by treating with a herbicide,
   wherein the selected plant, plant part, plant cell or population comprises or is enriched for the mutation in the second target sequence, and optionally wherein the selected plant, plant part, plant cell or population does not comprise integrated T-DNA in their genome, or is enriched for plants, plant parts or plant cells that do not comprise integrated T-DNA in their genome.
57. The method of any one of embodiments 47-56, further comprising generating at least one plant embryo, plant part or plant from the cell, plant part or plant selected.
58. The method of any one of embodiments 47-57, wherein said plant, plant part or plant cell is banana, coffee, or rice.
59. The method of embodiment 58, wherein said plant, plant part or plant cell is of a banana cultivar selected from the group consisting of Musa acuminata, Musa balbisiana, Musa itinerans, autotriploid Musa acuminata ‘Cavendish’, and autotriploid Musa acuminata ‘Gros Michel’.
60. A plant, plant part, plant cell or population thereof generated by the method according to any of embodiments 47-59.
61. The plant, plant part or plant cell population of embodiment 60, wherein at least 40%, optionally at least 55%, preferably at least 65% of the plant cells, plant parts or plants in the population resulting from the method do not comprise integrated T-DNA in their genome and comprise a mutation in the second gene.

In alternative aspects that may be combined with any embodiments herein, the invention provides a bacterium capable of transferring nucleotide sequences to a plant cell, comprising:
(a) a nucleotide sequence encoding vir genes, wherein the expression and/or activity of VirD5 is reduced or destroyed, and
(b) a T-DNA sequence encoding at least one DNA-editing agent, morphogene, antibiotic resistance gene, phosphite converting enzyme or RNAi construct. A preferred phosphite converting enzyme is PtxD. Preferred antibiotic resistance genes are NptII, HptII, Bla, Bar. Preferred morphogenes are WUS, BBM, LEAFY COTYLEDON1, LEAFY COTYLEDON2, Lec1.

### BRIEF DESCRIPTION OF THE FIGURES

FIG. 1—virD5 operon structure from: virA and virG activate the Ti plasmid repABC operon, elevating plasmid copy number in response to wound-released chemical signals. (Hongbaek Cho, Stephen C. Winans. Proceedings of the National Academy of Sciences. 2005, 102 (41) 14843-14848; DOI: 10.1073/pnas.0503458102)

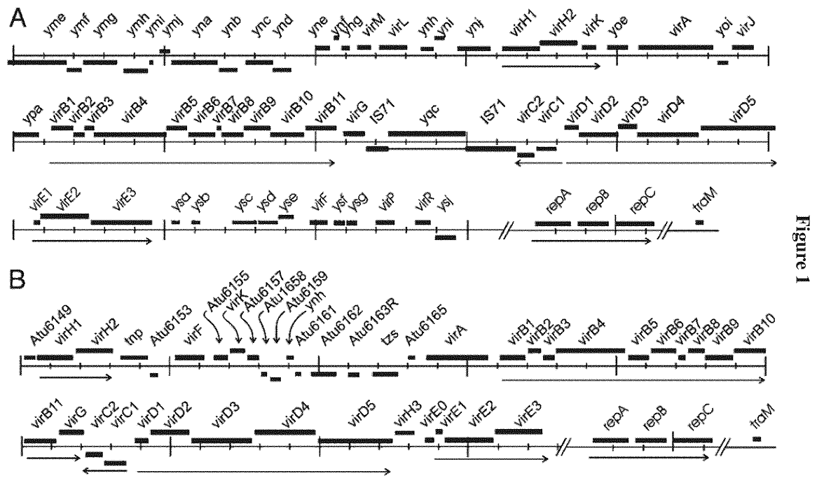

FIG. 2—Left upper panel depicts the nature of the virD5 mutagenic construct, with a 3′ and 5′ 500 bp fragment of virD5 sequence flanking a spectinomycin resistance cassette. The lower left panel shows the primer sequences used to validate the genotype of the EHA105virD5 mutant strain. Right panel is a DNA agarose gel (1%) with PCR fragments obtained from total genomic DNA from EHA105 and EHA105virD5 strains of Agrobacterium using oligos 569 and 571, and 569 and 570.

FIG. 3—Banana ECS expressing mcherry fluorescence assessed 10 days post-transformation with Agrobacterium strains (AGL1, EHA105 and EHA105virD5) carrying pMol_0630 (mcherry, cytidine base editor targeting ALS1).

FIG. 4—mCherry fluorescence in 2 month-old banana embryos transformed with WT Agrobacterium AGL1 carrying pMol_0630 (mcherry, cytidine base editor targeting ALS1), selected using 25 ug/L chlorsulfuron (CSF). Magnification 20×, bright field (BF) exposure 10 ms, fluorescence exposure 200 ms. Highlighted in boxes are non-fluorescent embryos.

FIG. 5—mCherry fluorescence in 2 month-old banana embryos transformed with WT Agrobacterium EHA 105 carrying pMol_0630 (mcherry, cytidine base editor targeting ALS1), selected using 25 ug/L chlorsulfuron (CSF). Magnification 20×, bright field (BF) exposure 10 ms, fluorescence exposure 200 ms. Highlighted in boxes are non-fluorescent embryos.

FIG. 6—mCherry fluorescence in 2 month-old banana embryos transformed with Agrobacterium EHA 105virD5 carrying pMol_0630 (mcherry, cytidine base editor targeting ALS1), selected using 25 ug/L chlorsulfuron (CSF). Magnification 20×, bright field (BF) exposure 10 ms, fluorescence exposure 200 ms

FIG. 7—T-DNA detection in 39 banana plantlets transformed with pMOL_0630; 7 from Agrobacterium strain AGL1, 11 from EHA105 and 20 from EHA105virD5, plus 1 WT shoot. Low ΔCt values (<25) indicate presence of T-DNA genes in the plant tissue, strongly indicating T-DNA integration (transgenic plant tissue). A threshold (>25 cycles) was set based on WT controls for each individual primer set. Data are normalized using a housekeeping gene to account for minor DNA concentration differences. High/absent ΔCt value (marked with *) indicates absence of T-DNA in the tissue (non-transgenic).

FIG. 8—Collated data for presence of T-DNA and ALS1 editing in banana plants transformed with Agrobacterium AGL1, EHA105 or EHA105virD5 carrying pMol_0630 (mcherry, cytidine base editor targeting ALS1), selected using 25 ug/L chlorsulfuron (CSF).

FIG. 9—Summary of editing and transgenesis detected in banana plants transformed with Agrobacterium EHA105 or EHA105virD5 carrying pMol_0926 (cytidine base editor targeting ALS1 and PPO2), and pMOL_0931 (cytidine base editor targeting ALS2 and ACO1), all selected using 25 ug/L chlorsulfuron (CSF). Representation of the proportion of plants genotyped in example 4 that were found to have the following genotype: non-transgenic and edited in both ALS and a trait gene (Non-transgenic co-edited); non-transgenic and non-edited in the trait gene (Non-transgenic WT); transgenic and co-edited in ALS and a trait gene (Transgenic co-edited); or transgenic but not edited in the trait gene (Transgenic WT). The trait gene is either ACO1 or PPO2 depending on the construct used to transform each regenerated plant.

FIG. 10—Embryos recovered from banana plants transformed with Agrobacterium EHA105 or EHA105virD5 carrying pMOL_1936, all selected using CSF. Editing in the ALS2 locus was assessed using amplicon Sanger sequencing to detect the presence of HDR donor integration using primers annealing to the genomic region around ALS2. None of the EHA105 embryos showed evidence of editing in the ALS2 locus, but 1 out of 16 EHA105virD5 embryos showed evidence of HDR donor integration.

FIG. 11—T-DNA detection in banana embryo transformed with EHA105virD5 which showed evidence of HDR donor integration compared with T-DNA detection in banana embryo transformed with EHA105. Low ΔCt values (<27) indicate presence of T-DNA genes in the plant tissue, strongly indicating T-DNA integration (transgenic plant tissue). A threshold (>27 cycles) was set based on WT controls for each individual primer set. Data are normalized using a housekeeping gene to account for minor DNA concentration differences. High/absent ΔCt value (marked with *) indicates absence of T-DNA in the tissue (non-transgenic).

### LIST OF SEQUENCES

SEQ ID NO: 1-ACO1 from Musa acuminata (Ma01_g11540.1)
SEQ ID NO: 2-ACO from Musa acuminata (Ma07_g19730.1)
SEQ ID NO: 3-ACS from Musa acuminata (Ma04_g31490.1)
SEQ ID NO: 4-ACS from Musa acuminata (Ma04_g35640.1)
SEQ ID NO: 5-ACS from Musa acuminata (Ma09_g19150.1)
SEQ ID NO: 6-PPO2 from Musa acuminata (Ma07_g03540)
SEQ ID NO: 7-F1_D5_F oligonucleotide for amplifying EHA105 virD5 gene sequence
SEQ ID NO: 8-F1_D5_R oligonucleotide for amplifying EHA105 virD5 gene sequence
SEQ ID NO: 9-F2_D5_F oligonucleotide for amplifying spectinomycin resistance cassette
SEQ ID NO: 10-F2_D5_R oligonucleotide for amplifying spectinomycin resistance cassette
SEQ ID NO: 11-F3_D5_F oligonucleotide for amplifying EHA105 virD5 gene sequence
SEQ ID NO: 12-F3_D5_R oligonucleotide for amplifying EHA105 virD5 gene sequence
SEQ ID NO: 13-569 oligonucleotide for screening for insertion in virD5 loci
SEQ ID NO: 14-570 oligonucleotide for screening for insertion in virD5 loci
SEQ ID NO: 15-571 oligonucleotide for screening for insertion in virD5 loci
SEQ ID NO: 16-G0390 oligonucleotide for qPCR of ACTIN
SEQ ID NO: 17-G0391 oligonucleotide for qPCR of ACTIN
SEQ ID NO: 18-G0327 oligonucleotide for qPCR of mCherry
SEQ ID NO: 19-G0328 oligonucleotide for qPCR of mCherry
SEQ ID NO: 20-G0418 oligonucleotide for qPCR of nCas9 5′
SEQ ID NO: 21-G0419 oligonucleotide for qPCR of nCas9 5′
SEQ ID NO: 22-G0422 oligonucleotide for qPCR of nCas9 3′
SEQ ID NO: 23-G0423 oligonucleotide for qPCR of nCas9 3′
SEQ ID NO: 24-GO117 oligonucleotide for sequencing of ALS1
SEQ ID NO: 25-GO118 oligonucleotide for sequencing of ALS1
SEQ ID NO: 26-GO163 oligonucleotide for sequencing of PPO2
SEQ ID NO: 27-GO164 oligonucleotide for sequencing of PPO2
SEQ ID NO: 28-3022 oligonucleotide for sequencing of ACO1
SEQ ID NO: 29-1757 oligonucleotide for sequencing of ACO1
SEQ ID NO: 30-33-exemplary primers for destruction or reduction of activity or expression of VirD5
SEQ ID NO: 34-GO975 oligonucleotide for sequencing ALS2
SEQ ID NO: 35-GO976 oligonucleotide for sequencing ALS2
SEQ ID NO: 36-G0534 oligonucleotide for qPCR of nCas9 5′
SEQ ID NO: 37-G0535 oligonucleotide for qPCR of nCas9 5′
SEQ ID NO: 38-G1158 oligonucleotide for qPCR of nCas9 3′
SEQ ID NO: 39-G1159 oligonucleotide for qPCR of nCas9 3′
SEQ ID NO: 40-G0989 oligonucleotide for qPCR of TaU6
SEQ ID NO: 41-G0990 oligonucleotide for qPCR of TaU6
SEQ ID NO: 42-G0390 oligonucleotide for qPCR of nptii
SEQ ID NO: 43-G0391 oligonucleotide for qPCR of nptii

### DETAILED DESCRIPTION OF THE INVENTION

The invention provides a bacterium capable of transferring nucleotide sequences to a plant cell, comprising: (a) a nucleotide sequence encoding vir genes, wherein the expression and/or activity of VirD5 is reduced or destroyed, and (b) a T-DNA sequence encoding at least one site-specific DNA-editing agent operable to introduce at least one mutation in at least one target sequence in a plant cell.

### Nucleotide Sequence Encoding Vir Genes

The bacteria of the invention comprise a nucleotide sequence encoding vir genes, and a T-DNA sequence, which enable the bacteria to transfer nucleotide sequences to a plant cell. In certain embodiments, the vir genes are encoded by a Vir-helper plasmid, a Ti plasmid or a Ri plasmid, but they may alternatively be encoded by a different plasmid or replicon or bacterial chromosome. The vir genes may be present in a virulence region (vir region) of the nucleotide sequence. In certain embodiments, the nucleotide sequence encoding vir genes also comprises a conserved DNA region known as the repABC gene cassette. The Ti or Ri plasmid described herein may be a modified version thereof, e.g. containing the vir genes that originated from the Ti or Ri plasmid, but lacking other features present in the original Ti or Ri plasmid (such as the T-DNA). According to some embodiments, the T-DNA sequence and the nucleotide sequence encoding the vir genes are present on two separate vectors, such as, but not limited to, a T-Binary vector and a ‘vir helper plasmid’ of a T-Binary system.

### T-DNA Sequence

The bacteria of the invention also comprise a T-DNA sequence. As used herein, a “T-DNA sequence” is a specific nucleotide sequence that forms the transfer DNA which is transferred into host plant cells. In certain embodiments, the nucleotide sequence encoding the vir genes also comprises the T-DNA sequence. In certain embodiments, the nucleotide sequence encoding the vir genes does not include a T-DNA sequence, and the T-DNA sequence is encoded by an additional plasmid. The additional plasmid may encode one or more additional elements selected from the group consisting of: a plant selectable marker, a bacterial selectable marker, a reporter gene, and at least one bacterial origin of replication. The T-DNA sequence may comprise left and right borders comprising conserved 25 bp imperfect repeats sequences. It is the sequence between the borders that is transferred during transformation.

In certain embodiments, the T-DNA sequence also encodes one or more additional elements, such as, but not limited to, selectable markers. Suitable selectable markers include antibiotic resistance genes, phosphite converting enzymes, morphogenes, and RNAi constructs. A preferred phosphite converting enzyme is PtxD. Preferred antibiotic resistance genes are NptII, HptII, Bla, Bar. Preferred morphogenes are WUS, BBM, LEAFY COTYLEDON1, LEAFY COTYLEDON2, Lec1. Examples of antibiotic selection markers that can be used are, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes. Further preferred markers include: mutant psbA that provides triazine-resistance, in particular G264S and I219V; and mutant enolpyruvylshikimate-3-phosphate synthase (EPSPS) that confer resistance to EPSP synthase inhibitors, in particular tryptophan 102 mutations, alanine 103 mutations and proline 106 mutations.

In preferred embodiments, the T-DNA sequence also encodes a marker that allows cells expressing the T-DNA sequence to be identified. In certain embodiments the marker is a fluorescent protein, which is shown to be particularly effective in the Examples, such as mCherry, mTurquoise 2, GFP, such as sfGFP or pH-tdGFP, Gamillus, mNeonGreen, mEYPF, mCitrine, Citrine, or TagRFP. Preferably the marker is mCherry.

The nucleotide sequences used in the invention will generally comprise a promoter. A promoter is a DNA sequence that is capable of controlling (initiating) transcription in a cell. As used herein, the “promoter” used to drive gene expression on the T-DNA is plant-expressible, i.e. capable of inducing, conferring, activating or enhancing expression in a plant cell, tissue or organ. This includes promoters of plant origin [e.g., T-DNA gene promoters, developmental-specific promoters, tissue specific promoters (e.g., mesophyll-specific promoters), seed-specific promoters, constitutively active promoters (e.g., Ubil, Uepl, or Actl), or organ-specific promoters (e.g., stem-, leaf-, root-, tuber-, stolon-, tricorne-, ovule-, anther-, pollen-, pollen tube-, sepal-, or pistil-specific promoters)], as well as promoters of non-plant origin that are capable of directing transcription in a plant cell (e.g promoters of viral or bacterial origin, such as the CaMV35S promoter). The promoter can include a constitutive promoter, or the promoter can include an inducible promoter. Examples of constitutive promoters are Cauliflower mosaic virus (CaMV) 35S promoter, a nopaline synthase promoter, or an octopine synthase promoter. Examples of tissue specific or inducible promoters are a napin promoter, a phaseolin promoter, a PTA29 promoter, a PTA26 promoter, a PTA13 promoter, an XVE estradiol-inducible promoter, or an ethanol-inducible promoter. Examples of promoters useful in the invention include, but are not limited to, Actin, CANV 35S, CaMV19S, GOS2. A preferred promoter is CsVMV. The promoter may be a Pol3 promoter. The nucleotide sequences of the agents to be expressed may be optimised for plant expression. Examples of such sequence modifications include, but are not limited to, an altered G/C content to more closely approach that typically found in banana, and the removal of codons atypically found in the plant species commonly (referred to as codon optimisation). In certain embodiments, the promoter is operably linked to the coding sequence. A promoter that is “operably linked” to a structural coding sequence can effectively control expression of the structural coding sequence. Thus, a structural coding sequence is “operably linked” to a promoter in a cell when RNA polymerase is able to transcribe the coding sequence into RNA. In certain embodiments, multiple promoters are used.

### Additional Plasmid

In certain embodiments, the nucleotide sequence encoding vir genes does not include a T-DNA cassette and the T-DNA is encoded by an additional plasmid.

Systems in which T-DNA and vir genes are located on separate replicons are called T-DNA binary systems. The additional plasmid may be otherwise known as a T-DNA binary vector. Examples of binary vectors are pICSL4723, pBIN19, pBHOI, pBinAR, pGPTV, pCAMBIA, pBIB-HYG, pBecks, pGreen or pPZP (Hajukiewicz, P. et al., Plant Molecular Biology, 25, 989 (1994), and Hellens et al. Trends in Plant Science 5, 446 (2000)).

In certain embodiments, the additional plasmid encodes one or more additional elements selected from the group consisting of: a plant selectable marker, a bacterial selectable marker, a reporter gene, and at least one bacterial origin of replication.

A suitable plant selectable marker may be an antibiotic selection marker. Examples of antibiotic selection markers that can be used are, neomycin phosphotransferase II (nptII) and hygromycin phosphotransferase (hpt). Additional marker genes which can be used include gentamycin acetyltransferase (accC3) resistance and bleomycin and phleomycin resistance genes. Further preferred markers include: mutant psbA that provides triazine-resistance, in particular G264S and I219V; and mutant enolpyruvylshikimate-3-phosphate synthase (EPSPS) that confer resistance to EPSP synthase inhibitors, in particular tryptophan 102 mutations, alanine 103 mutations and proline 106 mutations. The plant selectable marker may a fluorescent protein, such as mCherry, mTurquoise 2, GFP, such as sfGFP or pH-tdGFP, Gamillus, mNeonGreen, mEYPF, mCitrine, Citrine, or TagRFP.

A bacterial selectable marker ensures that bacteria which comprise the additional plasmid can be selected for, so plants are only transformed with bacteria which have the additional plasmid (and hence the T-DNA). Suitable markers may include an antibiotic selection marker. Examples of antibiotic selection markers include resistance to one or more of ampicillin, carbenicillin, chloramphenicol, gentamicin, G418, kanamycin, nalidixic acid, novobiocin, rifampicin, spectinomycin, streptomycin, tetracycline, trimethoprim and zeocin. In certain embodiments, the selectable marker is spectinomycin resistance.

Reduction or Destruction of VirD5 Expression and/or Activity

The expression and/or activity of VirD5 encoded by the nucleotide sequence encoding vir genes, preferably a Vir-helper plasmid, of the bacteria of the invention is reduced or destroyed. In certain embodiments, the expression and/or activity of VirD5 encoded by the nucleotide sequence encoding the vir genes is reduced. As used herein, the term “reduced” in the context of expression of VirD5 can refer to a reduction in expression of VirD5 by 10%, 20%, 30%, 40%, 50%, 60%, 70%, 80%, 90%, or completely. As used herein, the term “reduced” in the context of activity of VirD5 can refer to a reduction in activity of VirD5 by 10%, 20%, 30%, 40%, 50%, 60%, 70%, 80%, 90%, or completely. In preferred embodiments, the expression and/or activity of VirD5 encoded by the nucleotide sequence encoding the vir genes is destroyed.

In certain embodiments, the reduction or destruction of the expression and/or activity of VirD5 encoded by the nucleotide sequence encoding the vir genes is mediated by at least one mutation in the sequence encoding VirD5. Said at least one mutation in the sequence encoding VirD5 may be selected from the group consisting of: (a) at least one nucleotide insertion; (b) at least one nucleotide deletion; (c) an insertion-deletion (indel); (d) an inversion; (e) at least one nucleotide substitution; and (f) any combination of (a) to (e); wherein optionally the insertion or deletion is a frame shift insertion or deletion. In certain embodiments, said at least one mutation in the sequence encoding VirD5 is at least one nonsense or missense nucleotide substitution. In certain embodiments, said at least one mutation in the sequence encoding VirD5 is at least one nucleotide substitution that introduces a substitution in the encoded amino acid sequence that reduced or destroys activity. In certain embodiments, said at least one mutation in the sequence encoding VirD5 is an insertion and the inserted sequence encodes a selectable marker. The selectable marker may aid selection of bacteria that have the selectable marker inserted in the VirD5 sequence. Suitable markers may include an antibiotic selection marker. Examples of antibiotic selection markers include resistance to one or more of ampicillin, carbenicillin, chloramphenicol, gentamicin, G418, kanamycin, nalidixic acid, novobiocin, rifampicin, spectinomycin, streptomycin, tetracycline, trimethoprim and zeocin. In certain embodiments, the selectable marker is spectinomycin resistance.

In certain embodiments, the mutation that reduces or destroys VirD5 expression or activity is in the endogenous VirD5 gene and the bacterium does not comprise any exogenously provided VirD5 or an exogenously provided sequence encoding VirD5. As used herein, the term “endogenous” means native to the genome of the bacterium.

In certain embodiments, the reduction or destruction of the expression of VirD5 encoded by the nucleotide sequence encoding the vir genes is mediated by expression of a silencing RNA targeting VirD5. As used herein, “silencing RNA” refers to any RNA molecule that has the ability to reduce or destroy the expression of VirD5 encoded by the nucleotide sequence encoding the vir genes. As used herein, “silencing RNA” includes, but is not limited to, non-coding RNA, such as a small interfering RNA (siRNA), a short hairpin RNA (shRNA), a microRNA (miRNA), a Piwi-interacting RNA (piRNA), a phased small interfering RNA (phasiRNA), a trans-acting siRNA (tasiRNA), a transfer RNA (tRNA), a small nuclear RNA (snRNA), a small nucleolar RNA (snoRNA), a long non-coding RNA (lncRNA), a ribosomal RNA (rRNA), a repeat-derived RNA, an autonomous transposable RNA, a non-autonomous transposable RNA, and an antisense RNA.

In certain embodiments, destruction or reduction of activity or expression of VirD5 is achieved by introducing mutations using the following primers:

Forward1
(SEQ ID NO: 30)
GCCTTTCACATTGGAATCAT
Reverse1
(SEQ ID NO: 31)
ATGGGAAGACCTCGTTGTCA
Forward2
(SEQ ID NO: 32)
AAGGTCGTCCACGATACTTT
Reverse2
(SEQ ID NO: 33)
GGCACTGCTGTCAAGAAATC

In certain embodiments, an in-frame deletion in VirD5 is used. Such a deletion may be generated by obtaining upstream and downstream fragments using primers with complementary sequences and then generating a single fragment in which VirD5 is substantially or completely deleted. The single fragment can then be transferred to cells, such as by electroporation.

In certain embodiments, VirD5 is deleted in its entirety.

### DNA-Editing Agents

The bacteria of the invention comprise a T-DNA sequence encoding at least one site-specific DNA-editing agent operable to introduce at least one mutation in at least one target sequence in a plant cell.

In certain embodiments, the site-specific DNA editing agent comprises an endonuclease selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease.

In certain embodiments, the site-specific DNA-editing agent comprises a CRISPR-associated endonuclease or a modified CRISPR-associated endonuclease and the T-DNA sequence also encodes one or more guide RNAs specific to the at least one target sequence in a plant cell. In certain embodiments, the site-specific DNA-editing agent comprises a CRISPR-associated endonuclease or a modified CRISPR-associated endonuclease selected from the group consisting of: a base editor, a prime editor, a Cas9 endonuclease, or an endonuclease selected from the group consisting of SpCas9, xCas9, SpCas9-NG, SaCas9, AsCpf1, LbCpf1, CjCas9, NmCas9, StCas9, TdCas9, eSpCas9, HypaCas9, Cas9-SpRY/SpG, Cas4-Cas1-Cas2 complex and MAD7.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site. Endonucleases allow for precision genetic engineering of eukaryotic genomes, such as plant genomes. In some embodiments, the endonuclease is inactivated and catalytically dead, such as in dCas9, as discussed further below.

As used herein, “meganucleases” are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, but the number of such naturally occurring meganucleases is limited. In order to overcome this challenge, mutagenesis and high throughput screening methods have been used to create meganuclease variants that recognize unique sequences. For example, various meganucleases have been fused to create hybrid enzymes that recognize a new sequence. Alternatively, DNA-interacting amino acids of the meganuclease can be altered to design sequence specific meganucleases (see e.g., U.S. Pat. No. 8,021,867). Meganucleases can be designed using the methods described in e.g. Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514. Alternatively, meganucleases with site-specific cutting characteristics can be obtained using commercially available technologies e.g. Precision Biosciences' Directed Nuclease Editor™ genome editing technology.

“Zinc finger nucleases” (or “ZFNs”) and “transcription-activator like effector nucleases” (or “TALENs”), as used herein, have proven to be effective at producing targeted double-stranded breaks (see Christian M, Cermak T, Doyle E L, et al. Targeting DNA double-strand breaks with TAL effector nucleases. Genetics. 2010; 186 (2): 757-761. doi: 10.1534/genetics.110.120717). ZFNs and TALENs restriction endonuclease technology utilizes a non-specific DNA cutting enzyme linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally, FokI has the advantage of requiring dimerization to have nuclease activity, which means that the specificity increases, because each nuclease partner recognizes a unique DNA sequence. To enhance this effect, FokI nucleases have been engineered that can only function as heterodimers and have increased catalytic activity. Such nucleases avoid the possibility of unwanted homodimer activity and increase specificity of the double-stranded break. Thus, to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerise to create a double-strand break. Repair of these double-stranded breaks through the non-homologous end-joining (or NHEJ) pathway often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have been successfully generated in cell culture by using two pairs of nucleases simultaneously (Carlson D F, Fahrenkrug S C, Hackett P B. Targeting DNA With Fingers and TALENs. Mol Ther Nucleic Acids. 2012; 1 (1): e3. Published 2012-1-24. doi: 10.1038/mtna.2011.5). In addition, when a fragment of DNA with homology to the targeted region is introduced in conjunction with the nuclease pair, the double-strand break or double-stranded break can be repaired via homologous recombination (HR) to generate specific modifications (Urnov, F., Miller, J., Lee, Y. et al. Highly efficient endogenous human gene correction using designed zinc-finger nucleases. Nature 435, 646-651 (2005). https://doi.org/10.1038/nature03556). Although the nuclease portions of both ZFNs and TALENs have similar properties, the difference between these engineered nucleases is in their DNA recognition peptide. ZFNs rely on Cys2-His2 zinc fingers, and TALENs on TALEs. Both of these DNA recognizing peptide domains have the characteristic that they are naturally found in combinations in their proteins. Cys2-His2 Zinc fingers are typically found in repeats that are 3 bp apart, and are found in diverse combinations in a variety of nucleic acid interacting proteins. TALEs, on the other hand, are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, for example, modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from, for example, Sangamo Biosciences™ (Richmond, CA). Methods for designing and obtaining TALENs are described in Reyon et al. Nature Biotechnology (2012) 30 (5): 460-465; Miller et al. Nature Biotechnology (2011) 29:143-148; Cermak et al. Nucleic Acids Research (2011) 39 (12): e82, and Zhang et al. Nature Biotechnology (2011) 29 (2): 149-153. A recently developed web-based program named “Mojo Hand” was introduced by Mayo Clinic for designing TAL and TALEN constructs for genome editing applications (can be accessed through www.talendesign.org).

As used herein, “homing endonucleases” are double-stranded DNases that have large, asymmetric recognition sites (12 to 40 base pairs (bp)) and coding sequences that are usually embedded in either introns or inteins (Belfort, M. and Roberts, R. J. (1997) Nucleic Acids Research, 25, 3379-3388). Introns are spliced out of precursor RNAs, while inteins are spliced out of precursor proteins (Dujon, B. et al. (1989) Gene, 82, 115-118; Perler, F. B. et al. (1994) Nucleic Acids Research, 22, 1125-1127). Homing endonucleases are named using conventions similar to those of restriction endonucleases with intron-encoded endonucleases containing the prefix, “I-” and intein endonucleases containing the prefix, “PI-” (Belfort, M. and Roberts, R. J. (1997) Nucleic Acids Research, 25, 3379-3388; Roberts, R. J. et al. (2003) Nucleic Acids Research, 31, 1805-1812). Homing endonuclease recognition sites are rare. For example, an 18-base pair (bp) recognition sequence will occur only once in every 7×10¹⁰ base pairs of random sequence. However, unlike restriction endonucleases, homing endonucleases tolerate some sequence degeneracy within their recognition sequence (Gimble, F. S. and Wang, J. (1996) Journal of Molecular Biology, 263, 163-180; Argast, M. G. et al. (1998) Journal of Molecular Biology, 280, 345-353). That is, single base changes do not abolish cleavage but reduce its efficiency to variable extents. As a result, their observed sequence specificity is typically in the range of 10 to 12 base pairs.

As used herein, a “CRISPR-associated endonuclease” (or “Cas”) refers to an endonuclease having an RNA-guided polynucleotide-editing activity and is one of the components of the CRISPR/Cas system for genome editing, which uses at least one additional component, a “guide RNA” (gRNA). In some embodiments of the invention, the “CRISPR-associated endonuclease” is a “Cas9 endonuclease” (or “Cas9”). According to some embodiments, the “CRISPR-associated endonuclease” may be any Cas9 known in the art, such as, but not limited to, SpCas9, SaCas9, FnCas9, NmCas9, StlCas9, BlatCas9 (Shota Nakade, Takashi Yamamoto & Tetsushi Sakuma (2017), Cas9, Cpf1 and C2c1/2/3-What's next?, Bioengineered, 8:3, 265-273, and references therein). In other embodiments, the “CRISPR-associated endonuclease” may be Cpf1, such as, but not limited to, AsCpf1 or LbCpf1 (Shota Nakade, Takashi Yamamoto & Tetsushi Sakuma (2017), Cas9, Cpf1 and C2c1/2/3—What's next?, Bioengineered, 8:3, 265-273, and references therein).

As used herein, a “modified CRISPR-associated endonuclease” (or “modified Cas”) refers to a Cas in which the catalytic domain has been altered and/or which are fused to additional domain. According to some embodiments, a “modified Cas” refers to a Cas which contains inactive catalytic domains (dead Cas, or dCas) and has no nuclease activity while still being able to bind to DNA based on gRNA specificity. According to some embodiments, a “modified Cas” refers to a Cas which has a nickase activity (“nCas9”), thus inducing a single strand break. In some embodiments, the modified CRISPR-associated endonuclease is a “modified Cas9 endonuclease”, possibly a catalytically inactive Cas9 (or “dCas9”) or a nickase Cas9 (“nCas9”). The dCas can be utilised as a platform for DNA transcriptional regulators to activate or repress gene expression by fusing the inactive enzyme to known regulatory domains. For example, the binding of dCas alone to a target sequence in genomic DNA can interfere with gene transcription. There are a number of publicly available tools available to help choose and/or design target sequences as well as lists of bioinformatically determined unique gRNAs for different genes in different species such as the Feng Zhang lab's Target Finder, the Michael Boutros lab's Target Finder “E-CRISP”, the RGEN Tools: “Cas-OFFinder”, the CasFinder: Flexible algorithm for identifying specific Cas9 targets in genomes and the CRISPR Optimal Target Finder.

Modified versions of the Cas enzyme containing a single inactive catalytic domain, either RuvC- or HNH-, are called ‘nickases’. With only one active nuclease domain, the Cas nickase cuts only one strand of the target DNA, creating a single-strand break or “nick”. A single-strand break or single-stranded break, or nick, is mostly repaired by single strand break repair mechanism involving proteins such as but not only, PARP (sensor) and XRCCI/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas nickase are treated as a double-strand break, in what is often referred to as a “double nick” CRISPR system. A double-nick which is basically non-parallel DSB can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas nickase to create a double-nick by designing two gRNAs with target sequences in close proximity and on opposite strands of the genomic DNA would decrease off-target effect as either gRNA alone will result in nicks that are not likely to change the genomic DNA, even though these events are not impossible.

In certain embodiments, a modified CRISPR-associated endonuclease is a base editor or a prime editor.

In certain embodiments, the site-specific DNA-editing agent comprises a base editor and the T-DNA sequence also encodes one or more guide RNAs specific to the target sequence or sequences. Base editing is a genome editing approach that uses components from CRISPR systems together with other enzymes to directly install point mutations into cellular DNA or RNA without making double-stranded DNA breaks. In particular, modified Cas, such as dCas9 or nCas9, can be used together with other enzymes (generally as a fusion protein) for base-editing. DNA base editors comprise a catalytically disabled nuclease fused to a nucleobase deaminase enzyme and, in some cases, a DNA glycosylase inhibitor. RNA base editors achieve analogous changes using components that target RNA. Base editors directly convert one base or base pair into another, enabling the efficient installation of point mutations in non-dividing cells without generating excess undesired editing by-products (Rees and Liu (2018), “Base Editing: Precision Chemistry on the Genome and Transcriptome of Living Cells”, Nature Reviews Genetics, 19 (12): 770-788). In certain embodiments, the base editor is a fusion polypeptide comprising a modified CRISPR-associated endonuclease and a deaminase moiety. In preferred embodiments, the modified CRISPR-associated endonuclease is nCas9 or dCas9 and the deaminase moiety is a cytidine deaminase moiety or an adenine deaminase moiety. In some embodiments, the base editor may optionally comprise a DNA glycosylase inhibitor. A preferred base editor is a fusion comprising nCas9 (D10A), cytidine deaminase APOBEC and uracil glycosylase inhibitor. Exemplary suitable base editors are provided in Zong et al. Nat Biotechnol. 2017, 35 (5): 438-440). In certain embodiments, the base editor has both adenine and cytidine deaminase activity and is a dual-deaminase base editor (Grünewald, et al., 2020, Nature Biotechnology, 38:861-864). Base editors contemplated include APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VQR-ABE, VRER-ABE, and SaKKH-ABE (Rees and Liu (2018), “Base Editing: Precision Chemistry on the Genome and Transcriptome of Living Cells”, Nature Reviews Genetics, 19 (12): 770-788, and references therein). Further contemplated base editors include modified Cas12 or Cas13, such as dCas12, dCas13, dCas12a, dCas12b, dCas13a, dCas13b, dCas13c, or dCas13d.

In certain embodiments, the site-specific DNA-editing agent comprises a prime editor and the T-DNA sequence also encodes one or more guide RNAs that are pegRNAs specific to the target sequence or sequences. Prime editing is a genome editing approach that can generate targeted insertions, deletions and point mutations. Prime editing uses a Cas9 nickase fused to a reverse transcriptase, which is programmed with a pegRNA that specifies the target site and the desired edit. In certain embodiments, the prime editor is a fusion polypeptide comprising a modified CRISPR-associated endonuclease, preferably nCas9, and a reverse transcriptase. The prime editor can be selected from the group consisting of PE2, PE2-VQR, PE2-VRQR, PE2-VRER, PE2-NG, PE2-SpG, PE2-SpRY, PE2*, PE3, PE3b, PE3b-CaMV, PE3b-retron, PE4, PE5, and PE+serine recombinase-Bxb1 integrase (Lin et al., Nat Biotechnol 38, 582-585 (2020), Anzalone et al., Nature 576, 149-157 (2019), Chen et al., Cell. 2021, 184 (22): 5635-5652). Derivatives of these prime editors may also be used.

Drag-and-Drop Genome Insertion without DNA Cleavage with CRISPR-Directed Integrases
Eleonora I. Ioannidi, Matthew T. N. Yarnall, Cian Schmitt-Ulms, Rohan N. Krajeski, Justin Lim, Lukas Villiger, Wenyuan Zhou, Kaiyi Jiang, Nathaniel Roberts, Liyang Zhang, Christopher A. Vakulskas, John A. Walker I I, Anastasia P. Kadina, Adrianna E. Zepeda, Kevin Holden, Jonathan S. Gootenberg, Omar O. Abudayyeh
bioRxiv 2021.11.01.466786; doi: https://doi.org/10.1101/2021.11.01.466786
Anzalone, A. V., Gao, X. D., Podracky, C. J. et al. Programmable deletion, replacement, integration and inversion of large DNA sequences with twin prime editing. Nat Biotechnol (2021). https://doi.org/e10.1038/s41587-021-01133-w).

In certain embodiments, the endonuclease is a prime-recombinase fusion.

In certain embodiments, the site-specific DNA-editing agent comprises a CRISPR-associated endonuclease or a modified CRISPR-associated endonuclease and the T-DNA sequence also encodes one or more guide RNAs specific to the target sequence or sequences and at least one donor template operable to introduce the at least one mutation via homology-dependent repair (HDR) or non-homologous end-joining (NHEJ).

Preferably, the endonuclease and any guide RNAs and donor templates are encoded by the T-DNA, the T-DNA is transferred to the plant cell and then the gene editing machinery is transiently expressed to mediate the introduction of mutations. In certain embodiments, the gene editing machinery is expressed by the bacterial cell and forms a ribonucleoprotein complex that is delivered to the plant cell.

In certain embodiments, the T-DNA sequence comprises at least one donor template operable to introduce the at least one mutation via homology dependent repair (HDR) or non-homologous end-joining (NHEJ). The T-DNA sequence may comprise at least one donor template which is flanked by sequences which can be recognized by guide RNA, also known as guide RNA sites or CRISPR-Cas9 sites. Preferably, the T-DNA sequence encodes a CRISPR-associated endonuclease or a modified CRISPR-associated endonuclease and one or more guide RNAs which are specific to the target sequence and specific to the guide RNA sites which flank the donor template, thereby resulting in donor release upon expression of the T-DNA sequence.

### Co-Editing

The examples demonstrate that the bacterium of the invention is effective for co-editing two or more target sequences at the same time. This allows for effective generation of plants carrying two or more specific mutations. Furthermore, a bacterium operable to introduce mutations in two or more target sequences enables highly efficient selection of gene-edited non-transgenic plants. This is because plants edited at the first target sequence are likely to also have been edited at the second sequence. For example, if one mutation introduces a selectable trait, this can be used to select plants that are enriched for the second mutation.

In certain embodiments, the T-DNA sequence encodes: (a) at least one CRISPR-associated or modified CRISPR-associated endonuclease, (b) a first guide RNA specific to a first target sequence, and (c) a second guide RNA specific to a second target sequence, wherein the at least one endonuclease is operable to introduce at least one mutation into the first target sequence and is operable to introduce at least one mutation into the second target sequence.

In certain embodiments, the at least one modified CRISPR-associated endonuclease is one or more base editors operable to introduce at least one mutation into the first target sequence and operable to introduce at least one mutation into the second target sequence.

In certain embodiments, the T-DNA sequence encodes: (a) at least one CRISPR-associated or modified CRISPR-associated endonuclease, (b) a first guide RNA specific to a first target sequence, and (c) at least one additional guide RNA specific to at least one additional target sequence other than the first target sequence, wherein the at least one endonuclease is operable to introduce at least one mutation into the first target sequence and is operable to introduce at least one mutation into the at least one additional target sequence.

In certain embodiments, the at least one modified CRISPR-associated endonuclease is one or more base editors operable to introduce at least one mutation into the first target sequence and operable to introduce at least one mutation into the at least one additional target sequence. According to certain embodiments, the same base editors is operable to introduce at least one mutation into the first target sequence and operable to introduce at least one mutation into the at least one additional target sequence via guide RNA sequences directed specifically at the first target sequence and the at least one additional target sequence.

In certain embodiments, (a) the T-DNA sequence also encodes a donor template operable to introduce at least one mutation via homology-dependent repair (HDR) or non-homologous end-joining (NHEJ) into the second target sequence; or (b) the T-DNA sequence also encodes a first donor template operable to introduce at least one mutation via homology-dependent repair (HDR) or non-homologous end-joining (NHEJ) into the first target sequence and a second donor template operable to introduce at least one mutation via homology-dependent repair (HDR) or non-homologous end-joining (NHEJ) into the second target sequence.

In certain embodiments, the at least one modified CRISPR-associated endonuclease is one or more prime editors and the first guide RNA is a first pegRNA specific to a first target sequence and operable to introduce at least one mutation into the first target sequence and the second guide RNA is a second pegRNA specific to a second target sequence and operable to introduce at least one mutation into the second target sequence.

In certain embodiments, the at least one CRISPR-associated endonuclease or modified CRISPR-associated endonuclease comprise at least two different endonucleases, wherein a first endonuclease is operable to introduce at least one mutation into the first target sequence, and a second endonuclease is operable to introduce at least one mutation into the second target sequence. The first endonuclease may not be operable to introduce any mutation into the second target sequence and/or the second endonuclease is not operable to introduce any mutation into the first target sequence. The first endonuclease may form a ribonucleoprotein complex with a first guide RNA specific for a first target sequence, and the second endonuclease may form a ribonucleoprotein complex with a second guide RNA specific for a second target sequence. For example, an endonuclease may be used that incorporates a specific type of guide RNA. For example, Cas9 uses both crRNA and trRNA to form an sgRNA, whilst Cas12a only requires a crRNA. Therefore, in certain embodiments, the first endonuclease is Cas9 or a modified Cas9 and the first guide RNA is a sgRNA, and the second endonuclease is Cas12a or a modified Cas12a and the second guide RNA is a crRNA, or the first endonuclease is Cas12a or a modified Cas12a and the first guide RNA is a crRNA, and the second endonuclease is Cas9 or a modified Cas9 and the second guide RNA is a sgRNA. Alternatively, or in combination, the first and second endonucleases may recognise different PAM sequences. In such embodiments the first and second endonucleases may be separately selected from SpCas9, CjCas9 and Cas12a.

In further embodiments, the first endonuclease may form a ribonucleoprotein complex with a sgRNA specific for a first target sequence and a second endonuclease may be a prime editor that forms a ribonucleoprotein complex with a pegRNA specific for a second target sequence. In further embodiments, the first endonuclease may be a prime editor that forms a ribonucleoprotein complex with a pegRNA specific for a first target sequence and the second endonuclease may form a ribonucleoprotein complex with a sgRNA specific for a second target sequence.

Accordingly, in some embodiments, the first and second endonucleases may recognise different PAM sequences. In some embodiments, the first and the second endonuclease are both operable to introduce at least one mutation into the first target sequence and/or are both operable to introduce at least one mutation into the second target sequence. The first and second endonucleases may recognise the same PAM sequence. In some embodiments, (i) first endonuclease is a base editor and the second endonuclease is a prime editor and the second guide RNA is a pegRNA, or wherein the first endonuclease is a prime editor and the first guide RNA is a pegRNA and the second endonuclease is a base editor; or (ii) the first endonuclease is a base editor and the second endonuclease is a CRISPR-associated endonuclease such as Cas9, or wherein the first endonuclease is a CRISPR-associated endonuclease such as Cas9 and the second endonuclease is a base editor. In embodiments employing a CRISPR-associated endonuclease such as Cas9, a donor template is generally used that is operable to introduce the at least one mutation via homology-dependent repair (HDR) or non-homologous end-joining (NHEJ).

In preferred embodiments, the at least one mutation introduced into the first target sequence results in a selectable trait in the plant cell. The selectable trait may be herbicide resistance, optionally wherein the at least one mutation includes at least one mutation in at least one acetolactate synthase (ALS) gene, wherein the at least one mutation in the ALS gene provides resistance to an ALS inhibitor. Without wishing to be bound by theory or mechanism, the selectable trait enables selection of cells, plant parts or plants edited in the second target sequence or in the at least one additional target sequence.

### Guide RNAs

The T-DNA may optionally encode one or more guide RNAs specific to the target sequence or sequences.

The term “guide RNA” as used herein refer to a polynucleotide which facilitates the specific targeting of a CRISPR-associated endonuclease or a modified CRISPR-associated endonuclease to a target sequence such as a genomic or episomal sequence in a cell. According to some embodiments, guide RNAs can be chimeric/uni-molecular (comprising a single RNA molecule, also referred to as single guide RNA or sgRNA) or modular (comprising more than one separate RNA molecule, typically a crRNA and tracrRNA which may be linked, for example by duplexing). According to some embodiments, a guide RNA is a sgRNA, which is an RNA molecule which includes both the tracrRNA and crRNA (and a connecting loop). The sgRNA comprises a nucleotide sequence encoding the target homologous sequence (crRNA) and the endogenous bacterial RNA that links the crRNA to the Cas nuclease (tracrRNA) in a single chimeric transcript. This region of the crRNA, known as the variable region, confers the cutting specificity of the associated endonuclease, and is typically 20 nucleotides in length. The guide RNA/Cas complex is recruited to the target sequence by the base-pairing between the gRNA sequence and the complement genomic DNA. For successful binding of some Cas molecules, such as Cas9, the genomic target sequence must also contain the correct Protospacer Adjacent Motif (PAM) sequence immediately following the target sequence. The binding of the guide RNA/Cas complex localises the Cas to the genomic target sequence. Wild type Cas9 can cut both strands of the DNA causing a double-strand or double-stranded break. Variants of Cas9 such as nickases, only cut a single strand. Guide sequences for base editors (nickase or catalytically dead Cas9 fused to a deaminase) target the deaminase to a specific region of the genome where specific nucleotides are edited in a window of activity within the sgRNA sequence. Just as with ZFNs and TALENs, the double-stranded breaks produced by CRISPR/Cas can be repaired by HR (homologous recombination) or NHEJ (non-homologous end-joining), and are susceptible to specific sequence modification during DNA repair. The Cas nuclease has two functional domains: RuvC and HNH, each cutting a different DNA strand. When both of these domains are active, the Cas causes double strand breaks in the genomic DNA. A significant advantage of CRISPR/Cas is the high efficiency of this system coupled with the ability to easily create synthetic guide RNAs. This creates a system that can be readily modified to target different genomic sites and/or to target different modifications at the same site. Additionally, protocols have been established which enable simultaneous targeting of multiple genes. The majority of cells carrying the mutation present biallelic mutations in the targeted genes. However, apparent flexibility in the base-pairing interactions between the guide RNA sequence and the genomic DNA target sequence allows imperfect matches to the target sequence to be cut by Cas.

According to some embodiments, a guide RNA is a prime editing RNA (pegRNA). The pegRNA is capable of capable of identifying the target site and providing the new genetic information to replace the target DNA nucleotides. The pegRNA may comprise an extended sgRNA sequence comprising the desired edit, a primer binding site and a reverse transcriptase template sequence. During genome editing, the primer binding site allows the 3′ end of the nicked DNA strand to hybridize to the pegRNA, while the reverse transcriptase template serves as a template for the synthesis of edited genetic information.

In certain embodiments, the sequence encoding one or more guide RNAs specific to the at least one target sequence in a plant cell may be operably linked to a Pol3 promoter. Examples of Pol3 promoters include, but are not limited to, AtU6-29, AtU626, AtU3B, AtU3d, and TaU6.

### Donor Template

As used herein, the term “donor oligonucleotide” or “donor template” refers to exogenous nucleotides, i.e. externally introduced into the plant cell to generate a precise change in the genome. The donor oligonucleotides may be synthetic. Preferably the donor oligonucleotides are encoded by the T-DNA sequence. In certain embodiments, the donor oligonucleotides are RNA oligonucleotides. In certain embodiments, the donor oligonucleotides are DNA oligonucleotides. In certain embodiments, the donor oligonucleotides comprise single-stranded donor oligonucleotides (ssODN), double-stranded donor oligonucleotides (dsODN), double-stranded DNA (dsDNA), double-stranded DNA-RNA duplex (DNA-RNA duplex) double-stranded DNA-RNA hybrid, single-stranded DNA-RNA hybrid, single-stranded DNA (ssDNA), double-stranded RNA (dsRNA), single-stranded RNA (ssRNA). In certain embodiments, the donor oligonucleotides are provided in a non-expressed vector format or oligo. In certain embodiments, the donor oligonucleotides comprise a DNA donor plasmid (e.g. circular or linearized plasmid). According to some embodiments, the donor template is encoded as part of a pegRNA which is used with a prime editor (e.g. at least part of the primer sequence of the pegRNA), optionally wherein the pegRNA is encoded by the T-DNA sequence.

In some embodiments, the donor templates introduce only minimal changes to the target sequence. In certain embodiments, the donor templates comprise 1-40, such as 5-40, 5-30, 5-20, 3-40, 3-30, 3-20, 5-15, 3-10, 10-30, or 10-20 nucleotide additions, deletions and substitutions relative to the target sequence.

According to some embodiments the donor template is designed to introduce a precise change in the genome through the Homology Dependant Repair (HDR) mechanism. Any appropriate design for HDR donor templates may be used. In certain embodiments, the donor templates comprise homology arms, such as two homology arms each of 10-1000 nucleotides in length, such as 100-1000, 200-1000, 200-800, 300-800, 300-700, 400-600 nucleotides. According to some embodiments the HDR donor template is released from a T-DNA. Donor flanking guide RNA sites may be encoded on the DNA delivered to the plant cell nucleus, to facilitate the donor release from the T-DNA.

According to other embodiments the donor template is designed to introduce a precise change in the genome through the Non Homologous End Joining (NHEJ) mechanism. Any appropriate design for NHEJ donor templates may be used. NHEJ donors do not require homology arms, as the donor is inserted in the genome at the site of a double stranded break. This mechanism requires the NHEJ donor to be released from a T-DNA, and therefore requires donor flanking guide RNA sites encoded on the DNA delivered to the plant cell nucleus.

In certain embodiments, the templates comprise chemical modifications, such as phosphorothioate modifications. Modifications could prevent ligation of linear donor and/or enhance its stability. According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides. According to a specific embodiment, the donor oligonucleotides comprising the ssODN (e.g. ssDNA or ssRNA) comprise about 200-500 nucleotides. According to further embodiments the donor oligonucleotides comprising the ssODN (e.g. ssDNA or ssRNA) comprise about 300-500 nucleotides or about 400-500 nucleotides. According to a specific embodiment, the donor oligonucleotides comprising the dsODN (e.g. dsDNA or dsRNA) comprise about 250-5000 nucleotides.

In certain embodiments, the donor templates are 40-200 nucleotides in length, such as 50-180, 60-150, 60-120, 70-120, 70-110, 80-110 or 80-100 nucleotides.

### Bacteria

A bacterium of the invention is capable of transferring nucleotide sequences to a plant cell nucleus. In certain embodiments, this capability may be achieved by transferring virulence genes and T-DNA to a bacteria that cannot otherwise transfer DNA to plants. Alternatively, the bacterium may be able to transfer nucleotide sequences to a plant if they naturally comprise virulence genes. Bacteria for use in the invention preferably comprise a type-IV secretion system.

In certain embodiments, the bacterium is of the genus Agrobacterium, the genus Rhizobium, the genus Ensifer, the genus Allorhizobium, the genus Neorhizobium, or the genus Shinella. These taxonomy groups are accurate at the date of filing. They may be subject to change over time if revisions to the taxonomy groups are made. Homologs of Agrobacterium's virulence proteins are found in some symbiotic plant-associated bacterial species belonging to the Rhizobium genus. For example, Rhizobium etli, encodes a complete set of virulence proteins and is able to mediate transfer and integration of DNA into a host-plant cell genome when provided with a T-DNA. Preferably, the bacterium is of the genus Agrobacterium. In some embodiments, the bacterium is selected from the group consisting of: Agrobacterium tumefaciens, Agrobacterium fabrum str. C58, Agrobacterium genomosp, Agrobacterium sp. S2/73, Agrobacterium sp. 13-2099-1-2, Agrobacterium sp. NCPPB 925, Agrobacterium rhizogenes, Agrobacterium salinitolerans, Agrobacterium vitis, Agrobacterium arsenijevicii, Agrobacterium deltaense, Agrobacterium larrymoorei, Rhizobium sp. AB2/73, Rhizobium sp. 16-488-2b, Rhizobium sp. 16-488-2a, Rhizobium sp. 16-449-1b, Rhizobium sp. L58/93, Rhizobium sp. L245/93, Rhizobium sp. E27B/91, Rhizobium sp. K1/93, Rhizobium sp. BK007, Rhizobium tumorigenes, Rhizobium skierniewicense, Rhizobium lusitanum, Neorhizobium sp. NCHU2750, Neorhizobium galegae, Ensifer sp. YR511, and Ensifer adhaerens. In some embodiments, the bacterium is selected from the group consisting of: Ensifer adhaerens, Ensifer adhaerens OV14, Ensifer alkalisoli, Ensifer aridi, Ensifer glycinis, Ensifer psoraleae, Ensifer sesbaniae, Ensifer shofinae, Ensifer sojae CCBAU 05684, Ensifer sp. 1H6, Ensifer sp. AP48, Ensifer sp. BR816, Ensifer sp. LC11, Ensifer sp. LC13, Ensifer sp. LC14, Ensifer sp. LC163, Ensifer sp. LC384, Ensifer sp. LC499, Ensifer sp. LC54, Ensifer sp. LCM 4579, Ensifer sp. M14, Ensifer sp. MMN_5, Ensifer sp. NM-2, Ensifer sp. OV372, Ensifer sp. Root1252, Ensifer sp. Root127, Ensifer sp. Root1298, Ensifer sp. Root1312, Ensifer sp. Root142, Ensifer sp. Root231, Ensifer sp. Root258, Ensifer sp. Root278, Ensifer sp. Root31, Ensifer sp. Root423, Ensifer sp. Root558, Ensifer sp. Root74, Ensifer sp. Root954, Ensifer sp. T2_8, Ensifer sp. USDA 6670, Ensifer sp. WSM1721, Ensifer sp. YR511, Ensifer sp. ZNC0028.

In preferred embodiments, the bacterium is Agrobacterium tumefaciens. The Agrobacterium tumefaciens bacterium may be derived from Agrobacterium tumefaciens strain EHA105 or Agrobacterium tumefaciens strain AGL1.

### Target Sequence and Mutations Therein

The site-specific DNA-editing agent is operable to introduce at least one mutation in at least one target sequence in a plant cell.

A “mutation”, as used herein, can mean at least one nucleotide insertion, at least one nucleotide deletion, an insertion-deletion (indel), an inversion, at least one nucleotide substitution, or any combination of the foregoing. The modification can result in a frameshift, a missense mutation, loss-of-function mutation, a nonsense mutation or a gain-of-function mutation in the target sequence. The agents operable to introduce the desired mutation can be designed according to the type of mutation that is required, using standard techniques.

In certain embodiments, the guide RNAs or pegRNAs are specific to the at least one target sequence.

The at least one target sequence may be in a gene. The at least one target sequence may be in a locus that encodes a silencing RNA.

In certain embodiments, the at least one target sequence may encode a protein that confers herbicide resistance, confers sensitivity to a pest, confers sensitivity to a pathogen, or is involved in stress tolerance, yield, growth rate, or yield quality.

The phrase “stress tolerance” as used herein refers to the ability of a plant to endure a biotic or abiotic stress without suffering a substantial alteration in metabolism, growth, productivity and/or viability. The phrase “abiotic stress” as used herein refers to the exposure of a plant, plant cell, or the like, to a non-living (“abiotic”) environmental, physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO2), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation. The phrase “biotic stress” as used herein refers to the exposure of a plant, plant cell, or the like, to a living (“biotic”) organism, yet including viruses, that has an adverse effect on metabolism, growth, development, propagation, yield or survival of the plant (collectively, “growth”). Biotic stress can be caused by, for example, bacteria, viruses, fungi, parasites, beneficial and harmful insects, weeds, and cultivated or native plants. The phrase “yield” or “plant yield” as used herein refers to increased plant growth (growth rate), increased crop growth, increased biomass, and/or increased plant product production (including grain, fruit, seeds, etc.). As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission. According to one embodiment, the pest is an invertebrate organism. Exemplary pests include, but are not limited to, insects, nematodes, snails, slugs, spiders, caterpillars, scorpions, mites, ticks, fungi, and the like. Identification of plant or pathogen target genes to be mutated may be achieved using any method known in the art such as by routine bioinformatics analysis.

In certain embodiments, the mutation in the target sequence may inactivate the gene or reduce its expression or activity.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, SK., Bae, E K., Lee, H. et al. Trees (2018) 32:823.), and increased drought tolerance in tomato by downregulation of SlbZIP38 (Pan Y et al. Genes 2017, 8, 402; doi: 10.3390/genes8120402. Any method known in the art for assessing increased yield may be used in accordance with the present invention. Exemplary methods of assessing increased yield include, but are not limited to, reduced DST expression in rice as described in Ar-Rafi Md. Faisal, et al, AJPS>Vol. 8 No. 9, August 2017 DOI: 10.4236/ajps.2017.89149; and downregulation of BnFTA in canola resulted in increased yield as described in Wang Y et al., Mol Plant. 2009 January; 2 (1): 191-200.doi: 10.1093/mp/ssn088. Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation (2017) 1,14-25. Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S Y et al. Rice (N Y). 2015; 8: 36; and reduce OMT levels in many plants, which result in altered lignin accumulation, increase the digestibility of the material for industry purposes as described in Verma S R and Dwivedi U N, South African Journal of Botany Volume 91, March 2014, Pages 107-125.

In certain embodiments, the at least one target sequence may encode an RNA or protein involved in a plant trait or a fruit trait, such as, but not limited to, ripening, metabolite levels, starch content, sugar content etc. In certain embodiments, the at least one target sequence may encode an RNA or a protein involved in fruit sweetness, fruit sugar content, fruit flavour, fruit ripening control, water stress tolerance, heat stress tolerance, or salt tolerance.

In some embodiments, the target sequence encodes a protein involved in fruit ripening, such as a protein involved in the production of ethylene. Wherein the plant is banana, in some embodiments, the target sequence may encode ACO (1-Aminocyclopropane-1-Carboxylic Acid Oxidase) and/or ACS (ACC-synthase). ACO and ACS are involved in the production of ethylene. In certain embodiments, desired mutations reduce expression or activity of ACO and/or ACS, which may delay ripening of the banana fruit. In preferred embodiments, a desired mutation introduces a stop codon in the sequence encoding ACO or ACS or both. In preferred embodiments, a target sequence is ACO1 (for example SEQ ID NO: 1, or a sequence having at least 90, 95, 98 or 99% sequence identity to SEQ ID NO: 1). In certain embodiments, the target is ACO and the target sequence is selected from SEQ ID NO: 1 and SEQ ID NO: 2, or sequence having at least 90, 95, 98 or 99% sequence identity to SEQ ID NO: 1 or SEQ ID NO: 2. In certain embodiments, a target sequence is ACS and the target sequence is selected from SEQ ID NO: 3, SEQ ID NO: 4 and SEQ ID NO: 5, or a sequence having at least 90, 95, 98 or 99% sequence identity to SEQ ID NO: 3, SEQ ID NO: 4 or SEQ ID NO: 5. Exemplary target sequences are also provided in WO 2018/220581.

In certain embodiments, the target sequence encodes a protein involved in fruit browning. Wherein the plant is banana, in some embodiments, the target sequence may encode PPO (polyphenol oxidase). PPO is the enzyme thought to be responsible for browning in banana. In preferred embodiments, the target sequence is PPO2 (for example, SEQ ID NO: 6, or a sequence having at least 90, 95, 98 or 99% sequence identity to SEQ ID NO: 6). Exemplary target sequences are also provided in WO 2018/220581. In certain embodiments, a desired mutation reduces expression or activity of PPO, which may delay ripening of the banana fruit. In preferred embodiments, a desired mutation introduces a stop codon in the sequence encoding PPO.

Optionally multiple sequences may be targeted for mutation (e.g. any combination of ACO, ACS and PPO genes could be targeted simultaneously) and, for each sequence target, one or a plurality of mutations could be introduced. The present invention can be used to mutate a great range of different genes to achieve various effects. The invention is particularly useful for modulation of endogenous genes to provide improved traits and to protect organisms against different biotic and abiotic stresses such as e.g. cancer, viruses, insects, fungi, nematodes, heat, drought, starvation etc. Preferably, the desired mutation reduces the expression of the gene or the activity of the expressed protein. Preferably, the mutations is the introduction of a stop codon.

In certain embodiments, the at least one mutation in at least one target sequence in a plant cell includes at least one mutation that results in a selectable trait in the plant cell, optionally wherein the selectable trait is herbicide resistance.

In some embodiments, the at least one mutation in at least one target sequence in a plant cell includes at least one mutation that results in a selectable trait in the plant cell. A “selectable trait” as used herein refers to a trait that can be selected for, for example when exposing the plant to a particular compound or exposing the plant to a particular environment, or when there is a physical property of the edit in the plant that can be observed. Such selection enables plants that have the desired mutation to be identified. Preferably, the selectable trait allows transient selection, i.e. selection is only able to occur when certain temporary conditions are present, such as exposing the plant to a particular compound. In preferred embodiments, the selectable trait is herbicide resistance. Examples of other selectable traits includes antibiotic resistance and phosphite selection. In certain embodiments, the “selectable trait” is a phenotypic outcome that can serve as a marker for transformation, for example by visual selection. For example, transient PDS gene editing will generate albino plants that can be selected.

In certain embodiments, the target sequence is an acetolactate synthase (ALS) gene. The at least one mutation in the ALS gene may provide resistance to an ALS inhibitor. The ALS gene may be acetolactate synthase 1 (ALS1) gene or the acetolactate synthase 2 (ALS2) gene in banana, wherein the plant cell is a banana cell. The at least one mutation in the ALS gene may be a substitution that introduces a substitution in the encoded amino acid sequence, preferably at Pro-187 in banana ALS1 or Pro-181 in ALS2, most preferably Pro187Ser in ALS1 or Pro181Ser in ALS2. In certain embodiments, the ALS inhibitor is a sulfonylurea, imidazolinone, triazolopyrimidine, pyrimidinyl oxybenzoate, or sulfonylamino carbonyl triazolinones, preferably wherein the ALS inhibitor is chlorsulfuron. Other examples of ALS inhibitors include a pyrimidinylthiobenzoate, a sulfonylaminocarbonyltriazolinone and a sulfonylurea. Preferred imidazolinones include imazamethabenz-methyl, imazamox, imazapic, imazapyr, imazaquin and imazethapyr. Preferred pyrimidinylthiobenzoates include bispyribac-sodium and pyrithiobac-sodium. Preferred sulfonylaminocarbonyltriazolinones include flucarbazone-sodium and propoxycarbazone-sodium. Preferred sulfonylureas include bensulfuron-methyl, chlorimuron-ethyl, chlorsulfuron, foramsulfuron, halosulfuron-methyl, mesosulfuron-methyl, metsulfuron-methyl, nicosulfuron, primisulfuron-methyl, prosulfuron, rimsulfuron, sulfometuron-methyl, sulfosulfuron, thifensulfuron-methyl, triasulfuron, tribenuron-methyl, trifloxysulfuron-sodium and triflusulfuron-methyl. Preferred triazolopyrimidines include cloransulam-methyl, diclosulam, florasulam, flumetsulam, penoxsulam and pyroxsulam. The ALS mutation(s) introduced may provide resistance to any one or more ALS inhibitors, for example a single mutation could provide resistance against a plurality of inhibitors and multiple mutations could provide resistance against different inhibitors.

### Plants

In some embodiments, the plant cell is of banana, coffee, or rice.

In preferred embodiments, the plant cell is of banana. As used herein, the term “banana” refers to a plant of the genus Musa, including plantains. These include Musa acuminata (e.g. Musa acuminata banksia, Musa acuminata Calcutta, and Musa acuminata DH-Pahang), Musa balbisiana, Musa itinerans, and autotriploid Musa acuminata ‘Cavendish’ and ‘Gros Michel’. According to a specific embodiment, banana is autotriploid Musa acuminata ‘Cavendish’. According to a specific embodiment, the banana is the Grand Nain cultivar of Musa acuminata ‘Cavendish’, preferably GN236. Cultivated bananas are infertile autotriploids (AAA) derived from the progenitor species Musa acuminata (genome AA). Additionally, plantains (AAB or ABB) are infertile interspecific allotriploids derived from the hybridisation of Musa acuminata (AA) and Musa balbisiana (genome BB). The triploid nature of cultivated banana and plantain prevents them from producing viable seeds, whereas wild species are diploid and can produce viable seeds. In particular embodiments, the banana plant is triploid. Other ploidies are contemplated, including diploid and tetraploid.

In certain embodiments, the plant cell is of coffee. As used herein, the term “coffee” refers to a plant of the genus Coffea. These include Coffea arabica, Coffea canephora, Coffea liberica, Coffea charrieriana and Coffea eugenioides.

In certain embodiments, the plant cell is of rice. As used herein, the term “rice” refers to a plant of the genus Oryza and Zizania. These include Oryza sativa, Oryza glaberrim, Oryza rufipogon, Oryza barthii and Oryza nivara.

### Plant Cells and Plant Parts

Plant cells include cells from a meristematic region, cells of a callus tissue, leave cells, root cells, shoot cells, somatic cells, flower cells, pollen cells, microspores, seed cells, embryonic cells, embryogenic cells, gametophytic cells, sporophytic cells, and somatic cells, and a combination of the foregoing. Protoplasts can be derived from any plant tissue. According to some embodiments, a plant cell is a cell of a suspension culture, such as of an Embryonic Cell Suspension (ECS).

“Plant parts”, as used herein, include differentiated and undifferentiated tissues including, but not limited to roots (including tubers), rootstocks, stems, scions, shoots, fruits, leaves, pollens, seeds, tumor tissue, and various forms of cells and culture (e.g. single cells, protoplasts, embryos, embryogenic cells, embryonic cells, cells of a meristematic region, cells of a callus tissue, leave cells, root cells, shoot cells, somatic cells, flower cells, pollen cells, microspores, protoplasts, and a combination of the foregoing). The plant tissue may be in plant or in a plant organ, tissue or cell culture. In particular embodiments, the “plant part” is a fruit, a leaf, a root, or plant vasculature (e.g. xylem). Fruit comprises tissues such as fruit flesh and fruit peel. In other embodiments, the “plant part” is a seed. The term “seed” as used herein refers to a unit of reproduction of a flowering plant capable of developing into another such plant.

### Methods

The invention provides a method of generating a plant, plant part, plant cell or population thereof comprising at least one mutation in at least one target sequence, the method comprising contacting a plant, plant part or plant cell with the bacterium of the invention, optionally wherein the method further comprises regenerating said cell or plant part to obtain a whole plant. According to preferred embodiments, the method provides a non-transgenic plant, plant part, plant cell or population thereof comprising at least one mutation in at least one target sequence. According to some embodiments, at least 50%, optionally at least 60%, or 70%, preferably at least 80% of the contacted plants, plant parts or plant cells are non-transgenic and comprise at least one mutation in at least one target sequence.

Preferably, the cells, plants, and plant parts described herein are non-transgenic. According to some embodiments, the methods disclosed herein result in a cell, plant or plant cell which is non-transgenic. In the context of plants and their parts and cells, “transgenic” means within the genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A non-transgenic plant does not comprise such a heterologous polynucleotide within its genome. By contrast, a change made by gene editing, as discussed herein, will be made to an endogenous sequence. As used herein, the term “endogenous” means native to the genome of the plant or plant cell, and at the native position within the genome. As used herein, “heterologous” polynucleotides are from a foreign species. In some preferred embodiments, especially where an endogenous sequence has been gene-edited, the plants of the invention contain no heterologous DNA.

“Contacting” as used herein encompasses transformation, whereby the bacterium introduces nucleic acid constructs into a cell. In preferred embodiments a suspension of Agrobacterium cells is mixed with an ECS. The mixture may then be incubated and then the bacteria suspension can be removed to provide ECS cells that can be cultured. In certain embodiments, a heat shock treatment is applied (for example at 45C for 5 minutes). Suitable methods for banana are described in, for example, Shivani and Tiwari, Scientia Horticulturae, 246:675-685). In alternative embodiments, infiltration into plant leaves is used.

In preferred embodiments, the genome of the plant, plant part or plant cell that is generated does not comprise any integrated T-DNA sequence. In some embodiments, the method further comprises selecting at least one plant cell, plant part or plant that comprises at least one mutation in the target sequence or sequences and does not comprise any integrated T-DNA sequence in its genome. Said selection may comprise genotyping.

In certain embodiments, at least one of the mutations in a target sequence confers a selectable trait to the plant cell, plant part or plant. In certain embodiments, the at least one mutation in at least one target sequence includes: (a) at least one mutation in a first target sequence which results in a selectable trait in the plant, plant part or plant cell; and (b) at least one mutation in a second gene. In some embodiments, the method further comprises selecting a cell, plant part or plant having the selectable trait. Such embodiments may comprise treating the cell, plant part or plant with a selection agent. In preferred embodiments, the selectable trait is herbicide resistance. In said embodiments, said selecting is by selecting cells, plant parts or plants which are herbicide resistant. Such embodiments may comprise treating the cell, plant part or plant with the herbicide. In preferred embodiments, the target sequence or first target sequence is an ALS gene and optionally the mutation provides herbicide resistance to ALS inhibitors. In said embodiments, said selecting is by selecting cells, plant parts or plants which are resistant to ALS inhibitors.

In certain embodiments, the ALS inhibitor for use in the methods of the invention is an imidazolinone, a pyrimidinylthiobenzoate, a sulfonylaminocarbonyltriazolinone, a sulfonylurea, or a triazolopyrimidine. Preferably the ALS inhibitor is a sulfonylurea. Preferred imidazolinones include imazamethabenz-methyl, imazamox, imazapic, imazapyr, imazaquin and imazethapyr. Preferred pyrimidinylthiobenzoates include bispyribac-sodium and pyrithiobac-sodium. Preferred sulfonylaminocarbonyltriazolinones include flucarbazone-sodium and propoxycarbazone-sodium. Preferred sulfonylureas include bensulfuron-methyl, chlorimuron-ethyl, chlorsulfuron, foramsulfuron, halosulfuron-methyl, mesosulfuron-methyl, metsulfuron-methyl, nicosulfuron, primisulfuron-methyl, prosulfuron, rimsulfuron, sulfometuron-methyl, sulfosulfuron, thifensulfuron-methyl, triasulfuron, tribenuron-methyl, trifloxysulfuron-sodium and triflusulfuron-methyl. Preferred triazolopyrimidines include cloransulam-methyl, diclosulam, florasulam, flumetsulam, penoxsulam and pyroxsulam. Most preferably, the ALS inhibitor for use in the methods of the invention is chlorsulfuron. The ALS mutation(s) introduced may provide resistance to any one or more ALS inhibitors, for example a single mutation could provide resistance against a plurality of inhibitors and multiple mutations could provide resistance against different inhibitors.

In preferred embodiments, the method comprises treatment of an embryo, or a population of embryos, such as culturing embryos on embryo development media comprising an ALS inhibitor. “Culturing embryos”, “incubating embryos” and “treating embryos” as used herein encompasses generating embryos from embryogenic cells and incubating developing embryos. In such embodiments, the embryos are preferably incubated on embryo development medium comprising an ALS inhibitor. This means that embryogenic cells are cultured on embryo development medium as they develop into embryos. In certain embodiments, the embryos are cultured until fully developed embryos are observed. In certain embodiments, the embryos are incubated on embryo development medium comprising an ALS inhibitor for 8-20 weeks, such as 8-16, 10-16, 10-14, 12-14 or around 13 weeks. In certain embodiments, the embryos are incubated on embryo development medium comprising an ALS inhibitor at a concentration of 10-80 μg/L, such as 10-70, 15-65, 15-55, 20-55, 20-50, 20-40, 20-30 or around 25 μg/L. In certain embodiments, the embryos are incubated on embryo development medium comprising an ALS inhibitor at a concentration of 10-80 μg/L, such as 20-70, 25-65, 30-60, 40-60, 40-55, 45-55 or around 50 μg/L. In certain embodiments the embryos are incubated on embryo development medium comprising an ALS inhibitor at a concentration of 10-80 μg/L, such as 10-70, 15-65, 15-55, 20-55, 20-50, 20-40, 20-30 or around 25 μg/L or a concentration of 10-80 μg/L, such as 20-70, 25-65, 30-60, 40-60, 40-55, 45-55 or around 55 μg/L, for 8-20 weeks, such as 8-16, 10-16, 10-14, 12-14 or around 13 weeks. In certain embodiments, the embryos or embryogenic cell suspension are allowed to recover following transformation and before ALS inhibitor selection, such as for 1-14, 3-10, 5-10 or around 7 days. Similar methods will also be appropriate for other selectable markers and selection agents.

In certain embodiments, the method comprises treatment of an embryogenic cell suspension. In certain embodiments, the embryogenic cell suspension is incubated in the presence of an ALS inhibitor for at least 3 days, such as at least 5, 10 or 15 days, optionally for less than 30 days, such as less than 25, 20, 15 or 10 days. In certain embodiments, the embryogenic cell suspension is incubated in the presence of an ALS inhibitor at a concentration of at least 500 μg/L, such as at least 1, 2, 3, 4, or 5 mg/L. In certain embodiments, the embryogenic cell suspension is incubated in the presence of an ALS inhibitor at a concentration of at least 500 μg/L, such as at least 1, 2, 3, 4, or 5 mg/L for at least 3 days, such as at least 5, 10 or 15 days, optionally for less than 30 days, such as less than 25, 20, 15 or 10 days. In certain embodiments, the method comprises treating banana embryos with an ALS inhibitor by culturing embryos, for example starting with cells from an ECS, in an embryo development medium comprising an ALS inhibitor. In certain such embodiments, the method comprises subsequently transferring embryos to an embryo development medium that does not comprise an ALS inhibitor. In certain embodiments, the method comprises culturing embryos from a cell suspension, cell population or cell mass in an embryo development medium comprising an ALS inhibitor, observing the development of embryos that are resistant to the ALS inhibitor, and physically separating those embryos from the remaining cells that are less developed or not developed. In certain embodiments, the developed embryos are transferred to an embryo development medium that does not comprise an ALS inhibitor. Similar methods will also be appropriate for other selectable markers and selection agents.

In certain embodiments, the invention comprises selecting at least one plant cell, such as an embryo, a plant tissue or plant that is resistant to treatment with an ALS inhibitor and physically separating them from cells, tissue or plants that are not resistant. In certain embodiments, the at least one selected cell, tissue or plant is transferred to a separate plate or growth medium, preferably without an ALS inhibitor.

In certain embodiments, treatment of cells with an ALS inhibitor, such as culturing embryos in the presence of an ALS inhibitor, provides a population of cells or embryos that will have been edited at an endogenous ALS gene and will also be edited at a target sequence and will comprise a desired mutation. In certain embodiments, at least 1%, 2%, 3%, 4% or 5% of cells or embryos comprise the mutation in the endogenous acetolactate synthase gene and comprise a desired mutation in a target sequence.

In certain embodiments, the method comprises treatment of a plant or plantlet. In certain embodiments, the method comprises spraying a plant or plantlet with an ALS inhibitor. In certain embodiments, spray additionally comprises an adjuvant, such as Silwett L-77. In certain embodiments, the plants or plantlets are sprayed with an ALS inhibitor at a concentration of at least 1 mg/L, such as at least 2, 3, 4 or 5 mg/L or 1-10 mg/L or 2-7 mg/L. In certain embodiments, the plants or plantlets are sprayed approximately once a week. In certain embodiments, the plants or plantlets are sprayed weekly for four weeks. In certain embodiments, the first weekly spraying is at the start of the first week. In certain embodiments, the plants or plantlets are sprayed four times, preferably once a week. In certain embodiments, the method comprises growing plants or plantlets in rooting medium to which an ALS inhibitor has been added. In certain embodiments, the rooting medium comprises an ALS inhibitor at less than 1 mg/L, such as less than 0.5 mg/L or less than 0.1 mg/L, such as 0.01-0.09 or 0.002-0.06 mg/L or about 0.005 mg/L. In certain embodiments the plantlets are grown in ALS inhibitor-containing rooting medium for 2-6 weeks, such as 3-5 weeks, around one month, or 4 weeks. In certain embodiments, the treatment comprises spraying the plants or plantlets and growing them in rooting medium comprising an ALS inhibitor. In certain embodiments, the plants or plantlets are exposed to an ALS inhibitor for 2-6 weeks, such as 3-5 weeks, around one month, or 4 weeks. In certain embodiments, the method comprises culturing embryos in the presence of an ALS inhibitor, preferably using the concentrations and/or time periods set out above, and does not comprise any other treatment with an ALS inhibitor. In such embodiments the method does not comprise treating plants or plantlets with an ALS inhibitor and does not comprise incubating an ECS with an ALS inhibitor. In such embodiments, the ECS following transformation is incubated in a medium that does not comprise an ALS inhibitor and plants and plantlets are grown in a medium that does not comprise an ALS inhibitor.

In certain embodiments the plant cell that is transformed is a protoplast. In preferred embodiments, the plant cell that is transformed is an embryonic cell, such as a cell in an embryogenic cell suspension.

In certain embodiments, at least 40%, optionally at least 55%, preferably at least 65% of the plant cells, plant parts or plants in the population resulting from the method do not comprise integrated T-DNA in their genome and comprise a mutation in the second gene.

In certain embodiments, the method comprises additional selection steps following selection with a selection agent, such as treatment using an ALS inhibitor. In particular, it may be useful to select embryos or plants that do not comprise heterologous DNA integrated into their genome and embryos or plants that are confirmed to comprise the at least one desired mutation in a target sequence. As discussed above, the bacteria and methods of the invention are useful for enriching for such plants and plant cells.

In certain embodiments, the method comprises, following selection using an ALS inhibitor or other selection agent, selecting at least one cell or plant that comprises the at least one desired mutation in a target sequence. The mutation may be at least one nucleotide insertion; at least one nucleotide deletion; at least one nucleotide substitution; and any combination of the foregoing. The selection of at least one cell or plant that comprises the at least one desired mutation in a target sequence may comprise detecting the presence of the mutated genome sequence.

In certain embodiments, the method comprises, following selection using an ALS inhibitor or other selection agent, selecting at least one cell or plant that does not comprise any heterologous DNA, in particular integrated nucleic acid constructs that encode the agents operable to introduce mutations. The selection of such at least one cell or plant may comprise confirming the absence of the nucleic acid construct sequence in the genome of the cell or plant.

This can be achieved using any technique known in the art capable of detecting the modification or editing event. such as, but not limited to, DNA sequencing (e.g. next generation sequencing), electrophoresis, an enzyme-based mismatch-detection assay, and a hybridisation assay such as PCR, RT-PCR, RNase protection, in-situ hybridisation, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing. High-resolution melting analysis is another method of validating the presence of an editing event. Yet another method is the heteroduplex mobility assay. Mutations can also be detected by analysing re-hybridised PCR fragments directly by native polyacrylamide gel electrophoresis (PAGE). This method takes advantage of the differential migration of heteroduplex and homoduplex DNA in polyacrylamide gels. Other methods of validating the presence of editing events are described in length in Zischewski (2017), Biotechnology Advances 1 (1): 95-104.

In preferred embodiments, T-DNA sequence additionally encodes a selectable marker, such as a fluorescent marker, such as mCherry. Such markers may aid in the selection of non-transgenic cells, for example, that do not have the T-DNA sequence integrated into their genome and only transiently expressed the editing agents, because they will not express the selectable marker, as demonstrated in the examples.

Suitable markers may include an antibiotic selection marker. Examples of antibiotic selection markers that can be used are, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes. Further preferred markers include: mutant psbA that provides triazine-resistance, in particular G264S and I219V; and mutant enolpyruvylshikimate-3-phosphate synthase (EPSPS) that confer resistance to EPSP synthase inhibitors, in particular tryptophan 102 mutations, alanine 103 mutations and proline 106 mutations. In preferred embodiments, the selectable marker is a fluorescent protein, which is shown to be particularly effective in the Examples, such as mCherry, mTurquoise 2, GFP, such as sfGFP or pH-tdGFP, Gamillus, mNeonGreen, mEYPF, mCitrine, Citrine, or TagRFP. Preferably the selectable marker is mCherry. The methods of the invention may therefore incorporate a step of detecting such a marker and selecting cells or plants that do not express the marker.

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended embodiments, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

When reference is made herein to particular sequences, these may also encompass sequences that substantially correspond to its complementary sequence as including minor sequence variations, resulting from, e.g. sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, “identifying” can include any technique known in the art capable of detecting the presence of the one or more silencing molecules, such as, but not limited to, DNA sequencing (e.g. next generation sequencing), electrophoresis, an enzyme-based mismatch-detection assay, and a hybridization assay such as PCR, RT-PCR, RNase protection, in-situ hybridization, primer extension, Southern blot, Northern Blot and dot blot analysis.

Unless otherwise defined, all technical and/or scientific terms used herein have the same meaning as commonly understood by one of ordinary skill in the art to which the invention pertains. Although methods and materials similar or equivalent to those described herein can be used, exemplary methods and/or materials are described. The materials, methods, and examples are illustrative only and are not intended to be limiting.

The terms “comprises”, “comprising”, “includes”, “including”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

All publications, patents and patent applications cited herein are hereby incorporated by reference in their entirety.

### EXAMPLES

### Example 1: Generating a virD5 Mutant in Agrobacterium tumefaciens EHA105

This example demonstrates the generation of a mutant virD5 Agrobacterium strain.

To investigate the effect of an Agrobacterium VirD5 strain on plant transformation, and regeneration of non-transgenic banana plants, a mutant of virD5 in Agrobacterium EHA105 was to be generated. Fortunately, the transcriptional unit of virD5 is encoded by an operon (virD1, D2, D3, D4, D5) in which virD5 is the final gene encoded, enabling the disruption of this gene without affecting the expression of downstream genes.

To effectively knock-out the function of the VirD5 protein in A. tumefaciens EHA105 a strategy of disrupting the virD5 gene with a spectinomycin resistance cassette was employed. To generate a mutagenic plasmid construct, 1000 bp of EHA105 virD5 gene sequence was amplified in two 500 bp halves using primers F1_D5_F and F1_D5_R (5′ fragment), and F3_D5_F and F3_D5_R (3′ fragment). These were assembled flanking a spectinomycin resistance cassette (amplified with primers F2_D5_F and F2_D5_F) for bacterial resistance, into the recipient vector pJQ200SK using Clontech in-fusion.

TABLE 1
Primers used in cloning and screening

| Oligo | |
|---|---|
| name | Oligo sequence |
| F1_D5_F | TCGAATTCCTGCAGCCCGGGATGACAGGAAAGTCGAAAGTT (SEQ |
| | ID NO: 7) |
| F1_D5_R | GTTAGCGGGGAGGGATCGATTTCTGCCAGAC (SEQ ID NO: 8) |
| F2_D5_F | GATCCCTCCCCGCTAACGCGGGC (SEQ ID NO: 9) |
| F2_D5_R | ATCCAGGCTGCTTAGTGCATCTAACGCTTGAGT (SEQ ID NO: 10) |
| F3_D5_F | CACTAAGCAGCCTGGATTGCCGATG (SEQ ID NO: 11) |
| F3_D5_R | GAACTAGTGGATCCCCCGGGTCAGCGTTTAAACGCTTTGTCTTGAG |
| | C (SEQ ID NO: 12) |
| 569 | TTAACTCCACCCGTGCGATC (SEQ ID NO: 13) |
| 570 | AATTCGTTTCAGGCGGCATG (SEQ ID NO: 14) |
| 571 | TGCTGGTTGGATTGAAGGTCA (SEQ ID NO: 15) |

TABLE 2
PCR conditions (CloneAmp HiFi PCR Premix)

| | Temperature | Time | Cycles |
|---|---|---|---|
| Denaturation | 98° C. | 10 sec | 30 |
| Annealing | 55° C. | 10 sec | |
| Extension | 72° C. | 10 sec | |
| | 4° C. | Hold | |

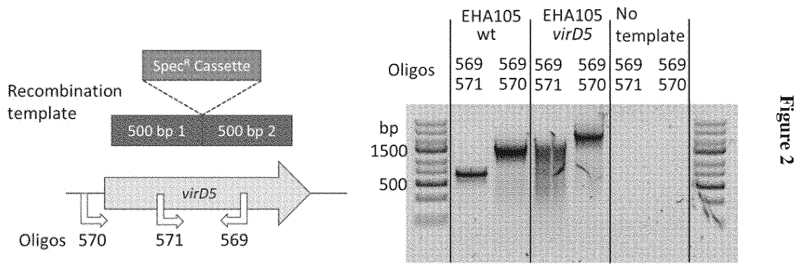

The resultant plasmid (pVIRD5specKO) cannot replicate in Agrobacterium, and so selecting for spectinomycin resistance will require the plasmid to integrate into the Agrobacterium genome via recombination. The plasmid was transferred into WT EHA105 cells by electroporation, and recombination events were selected using spectinomycin (100 μg/ml). Single colonies were picked and grown in LB media without antibiotics to allow secondary recombination to occur. These cells were diluted and plated on minimal media (AB minimal media plus 1.5% w/v agar) supplemented with 20% (w/v) sucrose, and 100 ug/ml spectinomycin. Several resultant colonies were streaked to single colonies on new sucrose/spectinomycin plates. Each new single colony was screened by PCR (primers 569+571 and 569+570) to identify a strain where there was no longer a WT band present in either primer set, indicating a complete insertion of the spectinomycin resistance cassette in every copy of the virD5 loci on the plasmid. This resultant strain was designated EHA105virD5 (FIG. 2).

### Example 2: The EHA105virD5 Strain is not Impaired Relative to WT EHA105 in Transient Expression of Mcherry Following Transformation of Banana ECS Cells

This example demonstrates that the EHA105virD5 strain is not impaired in the expression of genes from the T-DNA in the first few days following Agrobacterium-mediated transformation, termed ‘transient expression’. An mCherry fluorescent marker was used to assess expression 7-days post-transformation. Banana cultivar ECS Grand Nain GN236 was used as the germplasm for these experiments.

Agrobacterium tumefaciens preparation-Agrobacterium tumefaciens AGL1, EHA105 and EHA105virD5 carrying the binary plasmids pMol_0628 and pMol_0630 were streaked from glycerol in LB solid medium supplemented with rifampicin 50 mg/L, kanamycin 100 mg/L and carbenicillin 50 mg/L and incubated at 28° C. for 2 days. On the second day, the bacteria were inoculated in MG/L medium (discussed in Sarkar et al., Methods Mol Biol. 2013; 966:187-204) supplemented with the same antibiotics and incubated overnight at 28° C. Next day, the cultures were spun down at 4000 rpm for 10 minutes to pellet the cells and after they were resuspended in ABIM (Agrobacterium induction media; discussed in Sarkar et al., Methods Mol Biol. 2013; 966:187-204) to induce virulence and incubated overnight in the dark at room temperature. The OD₆₀₀ₙₘ of the bacteria was measured using a NanoDrop2000 and normalised to 0.6 in CS liquid media supplemented with acetosyringone (100 μM).

Banana ECS Agrotransformation—Three-day old cultures of Banana ECS were pelleted and resuspended in CS medium. 0.5 mL of the suspension (approximately 0.1 mL of settled cells of ECS) was split according to the number of transformations into small Eppendorf tubes. The cells were incubated at 45° C. for 5 min and then at room temperature for another 5 min. To this, 1 mL of agrobacterium suspension was added to each banana ECS sample. The mixtures were mixed well, spun down at 1000 rpm for 5 min and then incubated for 25 min at room temperature. The banana cells were pooled, and the bacteria suspension was removed. The cells were used to inoculate CS plates supplemented with acetosyringone 100 μM and incubated for 3 days at room temperature to promote transformation.

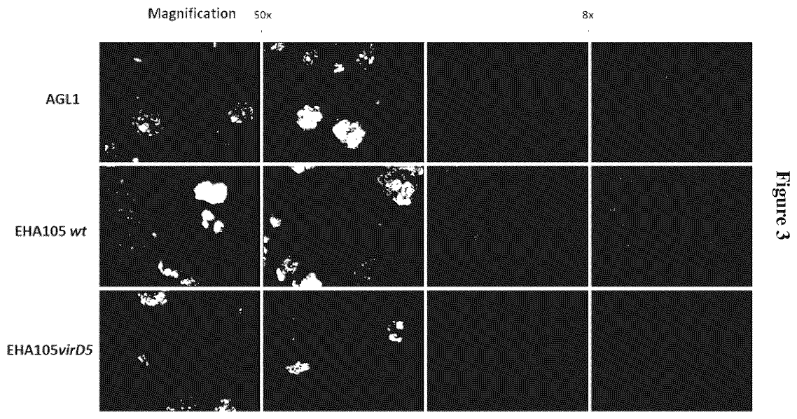

Fluorescence microscopy analysis—Banana ECS were checked for the presence of mCherry transient expression following 3 days of transformation using fluorescent microscopy (FIG. 3). Equal gain was used to take all images, and two magnifications were recorded (8 and 50×).

mCherry expression was observed in both EHA105 WT and EHA105virD5, with individual clusters of transformed cells showing bright expression in both lines, comparable to a control (AGL1). It was concluded that EHA105virD5 is not impaired in transient expression of elements from a T-DNA directly after transformation, relative to WT EHA105.

### Example 3: Transforming Cavendish Banana with the EHA105virD5 Strain Enriches Non-Transgenic Editing Events Compared to WT EHA105

This example demonstrates that the EHA105virD5 strain is capable of enriching populations of regenerated plants to contain high proportions of non-transgenic (genome does not comprise integrated exogenous DNA) gene editing events.

Banana ALS protein (acetolactate synthase) proline to serine (Pro187Ser) mutations in embryos and plants are resistance to the sulfonylurea compound chlorsulfuron. The cytidine base editor APOBEC described in Zong et al. Nat Biotechnol. 2017, 35 (5): 438-440) can be used to enact these changes in the banana genome and can be delivered to banana cells using a T-DNA based delivery system. Crucially, the T-DNA encoded base editor can edit the ALS loci either before, or after integration of T-DNA. Typically, a 10% non-transgenic banana, ALS edited, plant frequency is observed following the use of this system with WT Agrobacterium strains. The inventors determined that if the VirD5 protein is absent from the plant nucleus once T-DNA was present, rapid degradation of the T-DNA would be achieved, and therefore less integration events, and therefor fewer transgenic plants produced from each transformation. Importantly however, the non-transgenic, or transient editing rate was not expected to be affected—leading to an increased ratio of non-transgenic, ALS edited banana plants when the EHA105virD5 strain is used relative to the WT EHA105 strain for transformation.

Transformations were carried out as listed in Example 2 into Cavendish banana cultivar ECS Grand Nain GN236. Embryos and plants were produced from banana ECS material transformed with plasmids pMol_0628 (containing T-DNA expressing APOBEC base editor, non-targeting guide control and mCherry) and pMol_0630 (containing T-DNA expressing APOBEC base editor, ASL1 guide RNA to enact Pro187Ser mutation in banana ALS1 gene and mCherry), both without selection, and with 25 μg/L of chlorsulfuron added to the media during embryo development.

Banana embryos developing and germination—After 3 days, the banana ECS were collected from the previous media and transferred to CS media supplemented with cefotaxime 300 mg/L. One week later, the banana cells were transferred to EDM plates supplemented with cefo (cefotaxime) 300 mg/L (control), or cefo 300 mg/L and CSF 25 μg/L or cefo 300 mg/L and CSF 50 μg/L. The media was refreshed every 2 weeks until embryos developed. Subsequently, the banana embryos were transferred to maturation media without CSF (EMM—embryo maturation medium) for a month and then, they were transferred to germination medium (GM4) without CSF until shoots developed.

Plant development—Embryogenic cell suspensions (ECS) were transformed with an Agrobacterium strain harbouring plasmids encoding the nCas9 machinery and expressing sgRNAs. Agrobacterium transformation is performed according to Kanna et al., Molecular Breeding October 2004, Volume 14, Issue 3, pp 239-252. Embryogenic cells are co-cultivated with Agrobacterium for one to three days, and then transferred to embryo development medium (EDM) containing CSF and then a maturation medium without CSF (relevant media can be found, for example, in Strosse H., R. Domergue, B. Panis, J. V. Escalant and F. Côte, 2003, Banana and plantain 15 embryogenic cell suspensions (A. Vézina and C. Picq, eds). INIBAP Technical Guidelines 8, The International Network for the Improvement of Banana and Plantain, Montpellier, France). The mature embryos are germinated in germination medium (Strosse H., R. Domergue, B. Panis, J. V. Escalant and F. Côte. 2003. Banana and plantain embryogenic cell suspensions (A. Vézina and C. Picq, eds). INIBAP Technical Guidelines 8. The International 20 Network for the Improvement of Banana and Plantain, Montpellier, France) and young shoots are transferred to shoot maturation medium until approximately 1 cm in height. Shoots are transferred to rooting medium for plantlet development.

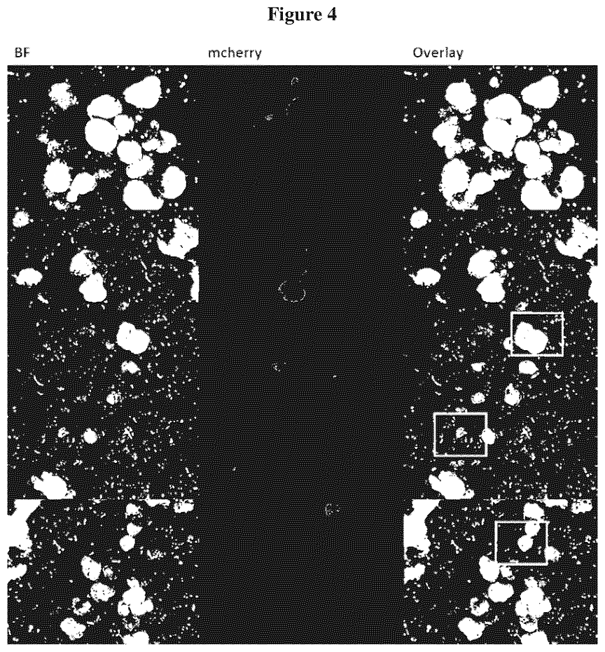

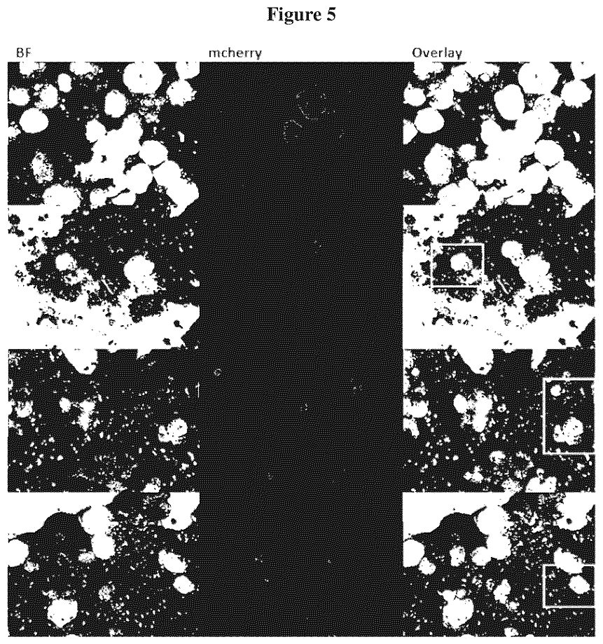

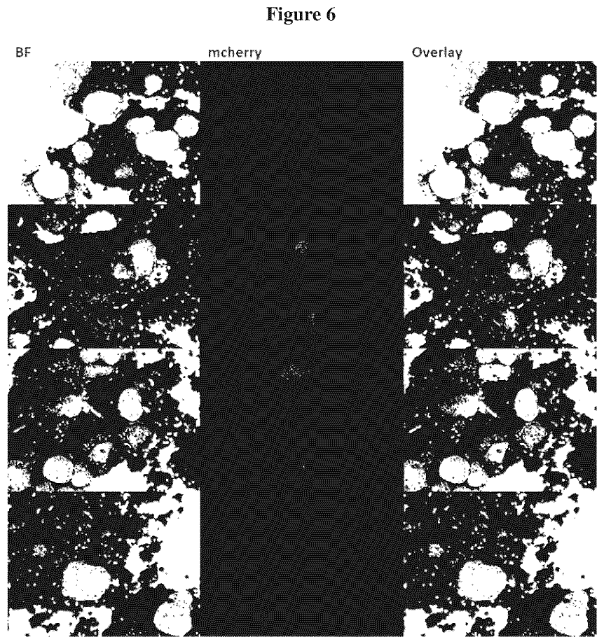

The mcherry fluorescence shown in FIGS. 4 to 6 demonstrates that there is a marked decrease in the ratio of fluorescent (putatively transgenic) and non-fluorescent (putatively non-transgenic) in EHA105virD5 relative to both EHA105 and AGL1 WT strains of Agrobacterium.

The number of CSF resistant embryos which developed following transformation with the three agrobacterium strains was examined (Table 3).

TABLE 3
Embryo frequency of banana ECS transformed using Agrobacterium
strain AGL1, EHA105 and EHA105virD5 with plasmid pMOL_0628
(negative control: mcherry, cytidine base editor with dummy guide)
and pMOL_0630 (mcherry, cytidine base editor targeting ALS1) and
selected on EDM media without selection, or with 25 μg/L chlorsulfuron.

| | No selection | 25 μg/L chlorsulfuron |
|---|---|---|
| AGL1 pMOL_0628 | +++++ | none |
| AGL1 pMOL_0630 | +++++ | +++ |
| EHA105 pMOL_0628 | +++++ | none |
| EHA105 pMOL_0630 | +++++ | +++ |
| EHA105virD5 pMOL_0628 | +++++ | none |
| EHA105virD5 pMOL_0630 | +++++ | + |

Without CSF selection a consistent number of regenerated embryos from all three Agrobacterium strains (AGL1, EHA105 and both resistant and non-resistant plasmid (pMOL_0628 and pMOL_0630) is observed. When CSF selection was applied, the control construct (no ALS sgRNA-pMOL_0628) did not support the regeneration of any CSF resistant, ALS base edited embryos, as expected. Both WT agrobacterium strains (AGL1 and EHA105) supported the regeneration of ˜200 CSF resistant, base edited embryos-however the EHA105virD5 transformed banana cells gave a reduced yield of CSF resistant, base edited embryos. This is consistent with a reduction in transgenic editing in a population of transformed cells using the EHA105virD5 strain. It was hypothesised that the EHA105virD5 strain-derived embryos were mostly ALS edited and non-transgenic.

To support these data, plants from this experiment were reproduced to genotype for ALS gene editing, and for the presence of T-DNA genes.

Plant genotyping—Plant tissue was sampled from leaf tissue in tissue culture, briefly, a 5 mm square piece of leaf was sampled and placed in a Qiagen collection microtubes (Cat. No. 19560). DNA was extracted using an Oktapure automatic platform using LGC sbeadex plant kit.

To detect the presence of transgenes in the banana plants genomic DNA qPCR using primer sets that bind to the T-DNA was used. Primer pairs G0418, G0419 and G0422, G0423 were used to detect the nCas9-PBE fusion, and primer pairs G0327, G0328 were used to detect mCherry. ACTIN was amplified using primers G0390 and G0391 from every sample and the amplification Ct value of this amplicon was used as a control (genomic copy number normalization).

TABLE 4
Primers used for T-DNA gDNA qPCR.

| qPCR primer | |
|---|---|
| target | Oligo name and sequence |
| ACTIN | G0390 ACCGAAGCCCCTCTTAACCC (SEQ ID NO: 16) |
| (normalisation) | G0391 GTATGGCTGACACCATCACC (SEQ ID NO: 17) |
| mCherry | G0327 AGTTCATGCGCTTCAAGGTG (SEQ ID NO: 18) |
| | G0328 ACAGCTTCAAGTAGTCGGGG (SEQ ID NO: 19) |
| nCas9 5′ | G0418 CCTCGTTGCTTGGGGAGTAG (SEQ ID NO: 20) |
| | G0419 GCCATCACCGAGTTCCTCAG (SEQ ID NO: 21) |
| nCas9 3′ | G0422 TCCGCTTCTTCTTTGGGCTC (SEQ ID NO: 22) |
| | G0423 AGGAGGTGGAGGAGGTCATC (SEQ ID NO: 23) |

Reactions were all set up with 5 μl of template DNA. Two WT embryo DNA controls, and a water control, were used. Applied PowerUp SYBR Green master mix was used to perform PCR in the Roche LightCycler96, according to the manufacturer's recommendations.

TABLE 5
PCR conditions for T-DNA gDNA qPCR

| | Temperature | Time | | Cycles | |
|---|---|---|---|---|---|
| UDG activation | 50° C. | 2 | min | Hold | |
| Polymerase activation | 95° C. | 2 | min | Hold | |
| Denaturation | 95° C. | 15 | sec | 40 | Continuous |
| Annealing | 60° C. | 1 | Min | | Acquisition |
| High resolution melting | 95° C. | 1 | min | | |
| | 40° C. | 1 | min | | |
| | 65° C. | 1 | s | | |
| | 97° C. | 1 | s | | |

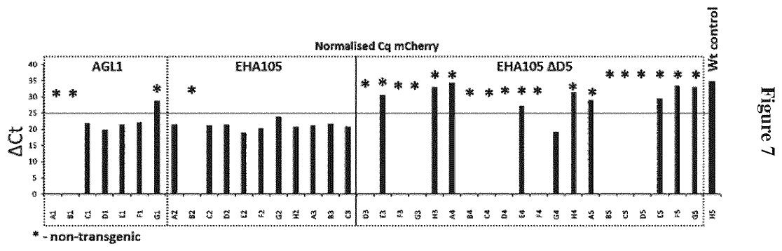

Raw data was analysed in Roche Lightcycler 96 software, and Ct values for each sample were collected, and exported to MS excel. The Ct values for the T-DNA amplicons were normalized using the ACTIN Ct levels. Once normalized, the data was plotted (FIG. 7).

Editing in the ALS1 loci was assessed using amplicon Sanger sequencing to detect the presence of cytidine-base editor induced mutations.

TABLE 6
Sequencing primers used to detect the presence of cytidine-base
editor induced mutations.

| Sequencing primers: | Oligo name and sequence |
|---|---|
| ALS1 (500 bp) | GO117 TCCATCAGGCTCTCACCCG (SEQ ID NO: 24) |
| | GO118 CCACCACCAACATAGAGGACT (SEQ ID NO: 25) |

TABLE 7
PCR conditions used for amplicon generation for Sanger sequencing

| | Temperature | Time | | Cycles |
|---|---|---|---|---|
| Initial Denaturation | 98° C. | 30 | sec | |
| Polymerase activation | 98° C. | 10 | sec | 35 |
| Denaturation | 65° C. | 15 | sec | |
| Annealing/Extension | 72° C. | 30 | sec | |
| Final Extension | 72° C. | 2 | min | |

| | |
|---|---|
| 4° C. | Hold |

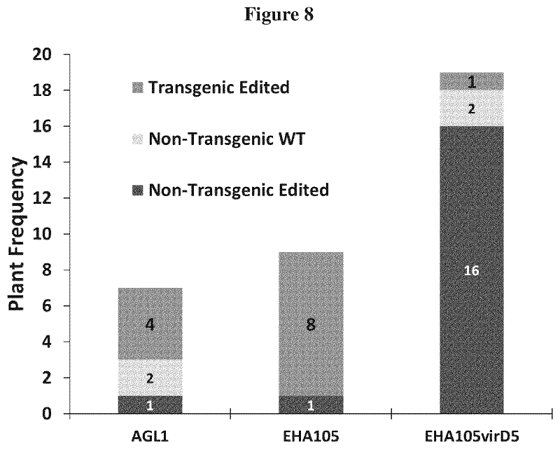

Amplicons were sent to Genewiz for Sanger sequencing. Geneious was used to assess the presence of C-T editing within the sgRNA target in each sample. Data for presence of T-DNA and ALS1 editing are summarised in FIG. 8. The EHA105virD5 transformed plants have a markedly different ratio of non-transgenic editing of ALS1 (84%) than plants transformed with EHA105 (11%) or AGL1 (14%) WT strains.

Hence, the EHA105virD5 strain is capable of enriching populations of regenerated plants to contain high proportions of non-transgenic gene editing-when ALS base editing is used as the means of selection.

### Example 4: The EHA105virD5 Strain of Agrobacterium Facilitates the Enrichment of Non-Transgenic, ALS and Trait Gene Edited Banana Plants

This example demonstrates that the EHA105virD5 strain is capable of enriching populations of regenerated plants to contain high proportions of non-transgenic gene editing events in ALS and a trait gene loci.

Banana ECS transformation, and plant regeneration was performed as detailed in previous examples. Table 8 details the plasmid constructs used in this Example. Plasmid pMOL0628 is a non-resistant mutation control (no ALS editing, but containing the cytidine base editor). Plasmids pMOL0630 and pMOL0632 encode the cytidine base editor and sgRNAs to generate CSF resistant edits in ALS1 and ALS2 respectively. These are control constructs to show CSF resistance and ALS base editing works as expected. The experimental constructs for this example were pMOL0926 (ALS1 base edit sgRNA as in pMOL0630 and a PPO2 sgRNA to generate a nonsense, premature stop codon mutation in MaPPO2) and pMOL0931 (ALS2 base edit sgRNA as in pMOL0632 and an ACO1 sgRNA to generate a nonsense, premature stop codon mutation in MaACO1). Banana line GN236 ECS was transformed using Agrobacterium strain EHA105 WT, and EHA 105virD5, and regenerated plants using 25 ng/mL CSF selection only during embryo development (EDM media).

TABLE 8
Details of transformation constructs and selection used.

| | | | Marker | |
|---|---|---|---|---|
| | | | edit | |
| Line | Plasmid | Description | target | Selection |
| GN_236 | control (no | — | — | 0 |
| | plasmid) | | | |
| | EHA105 | | | |
| GN_236 | pMOL_0628 | mcherry, sgGFP | | CSF 0, 25 ng/ml |
| GN_236 | pMOL_0630 | mcherry, sgALS1 | ALS1 | CSF 0, 25 ng/mL |
| GN_236 | pMol_0632 | mcherry, sgALS2 | ALS2 | CSF 0, 25 ng/ml |
| GN_236 | pMol_0926 | sgALS1, sgPPO2 | ALS1 | CSF 0, 25 ng/ml |
| GN_236 | pMol_0931 | sgALS2, sgACO1 | ALS2 | CSF 0, 25 ng/ml |
| | EHA105virD5 | | | |
| GN_236 | pMOL_0628 | mcherry, sgGFP | | CSF 0, 25 ng/ml |
| GN_236 | pMOL_0630 | mcherry, sgALS1 | ALS1 | CSF 0, 25 ng/ml |
| GN_236 | pMol_0632 | mcherry, sgALS2 | ALS2 | CSF 0, 25 ng/ml |
| GN_236 | pMol_0926 | sgALS1, sgPPO2 | ALS1 | CSF 0, 25 ng/ml |
| GN_236 | pMol_0931 | sgALS2, sgACO1 | ALS2 | CSF 0, 25 ng/ml |

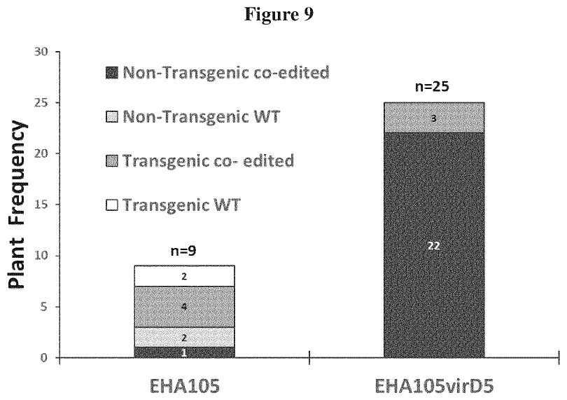

The first 44 plants to regenerate from these transformations were genotyped using the same methods detailed in Example 3, where tissue samples were taken from single leaves, DNA extracted using LGC Oktapure sbeadex technology, amplicons generated from Sanger sequencing for ALS1 and ALS2 as detailed previously and subjected to Sanger sequencing. Transgenic plants were identified using gDNA T-DNA qPCR as detailed in Example 3. Additional Amplicon PCR sequencing was performed for PPO2 and ACO1. Results are shown in FIG. 9. 88% non-transgenic, co-edited (ALS1/2 and trait gene; either ACO1 or PPO2) plant frequency was observed when the EHA 105virD5 strain of Agrobacterium was used. For ALS alone, 84% non-transgenic, ALS edited plant frequency was observed. This appears to be robust and reproducible.

TABLE 9
Sequencing primers used for amplicon PCR sequencing for PPO2 and ACO1

| Sequencing primers: | Oligo name and sequence |
|---|---|
| PPO2 | GO163 AGGTGCCCATGTTCTCGAAG (SEQ ID NO: 26) |
| | GO164 AAGTAGGTCACGTCCCTCCC (SEQ ID NO: 27) |
| ACO1 | 3022 TTGATGCCCACGAGCATTTGA (SEQ ID NO: 28) |
| | 1757 ATCCACTGGCCATCCTTGA (SEQ ID NO: 29) |

TABLE 10
PCR conditions used for amplicon PCR
sequencing for PPO2 and ACO1

| | Temperature | Time | | Cycles |
|---|---|---|---|---|
| Initial Denaturation | 98° C. | 30 | sec | |
| Polymerase | 98° C. | 10 | sec | 35 |
| activation | | | | |
| Denaturation | 65° C. | 15 | sec | |
| Annealing/Extension | 72° C. | 30 | sec | |
| Final Extension | 72° C. | 2 | min | |

| | |
|---|---|
| 4° C. | Hold |

TABLE 11
Collated genotyping data for the first 44 regenerated plants

| | | | | | | | | | % |
|---|---|---|---|---|---|---|---|---|---|
| | | | | | % | | | % | Non- |
| | | | | | Edited | | | Transgenic | transgenic |
| | | | Selection | Total | at | | # | edited | edited |
| | Target | Agro | (ug/L) | plants | ALS | # | Non- | in trait | in trait |
| Plasmid | gene(s) | Strain | CSF | genotyped | Locus | Transgenic | transgenic | gene | gene |
| pMOL | ALS1 | EHA105 | 25 | 3 | 100% | 0/3 | 3/3 | N/A | N/A |
| 0630 | | virD5 | | | | | | | |
| | | EHA105 | 25 | 1 | 100% | 1/1 | 0/1 | N/A | N/A |
| pMOL | ALS2 | EHA105 | 25 | 6 | 50% | 0/6 | 6/6 | N/A | N/A |
| 0632 | | virD5 | | | | | | | |
| | | EHA105 | 25 | 0 | N/A | N/A | N/A | N/A | N/A |
| pMOL | ALS1 | EHA105 | 25 | 9 | 88% | 6/9 | 1/9 | 67% | 11% |
| 0926 | and | | | | | | | | |
| | PPO2 | | | | | | | | |
| pMOL | ALS2 | EHA105 | 25 | 25 | 76% | 3/25 | 22/25 | 67% | 88% |
| 0931 | and | virD5 | | | | | | | |
| | ACO1 | | | | | | | | |

### Example 5: Editing Coffee ALS Using the EHA105virD5 Strain

EHA105virD5 Agrobacterium are used in addition to the regular strain of EHA105 to transform coffee embryogenic cali material using T-DNA plasmids detailed below.

### Example 6: Transforming Cavendish Banana with the EHA105virD5 Strain Allows for Non-Transgenic HDR Editing Events in Embryos

This example demonstrates that the EHA105virD5 strain facilitates the production of non-transgenic (genome does not comprise integrated exogenous DNA) HDR-edited embryos, while reducing the overall number of non-edited embryos. A donor-release HDR strategy was used.

Banana ALS2 protein (acetolactate synthase 2) proline to serine (Pro181Ser) mutations in embryos and plants are resistant to the sulfonylurea compound chlorsulfuron. These changes can be introduced using a donor molecule delivered to banana cells using a T-DNA based delivery system. The donor molecule is 1,088 bp in size and has homology to the ALS2 sequence barring the Pro181Ser change and 11 silent mutations within 71 bp of the Pro181Ser site. Either side of the donor module within the T-DNA are CRISPR-Cas9 sites to facilitate the donor “release” from the T-DNA.

Transformations were carried out as listed in Example 2 into Cavendish banana cultivar ECS Grand Nain GN218. Embryos were produced from banana ECS material transformed with four plasmids: pMOL_0628 (containing T-DNA expressing APOBEC base editor, non-targeting guide control and mCherry); pMOL_0630 (containing T-DNA expressing APOBEC base editor, ALS1 guide RNA to enact Pro197Ser mutation in banana ALS1 gene and mCherry); pMOL_1935 (containing T-DNA expressing mCherry, Cas9 (D10A) nickase, ALS2 P181S donor, 2xsgRNA targeting ALS2); pMOL_1936 (containing T-DNA expressing mCherry, Cas9 (D10A) nickase, ALS2 P181S donor with flanking sgRNA sites, 2xsgRNA targeting ALS2 and the donor flanking sites). All were tested without selection, and with 50 μg/L of chlorsulfuron added to the media during embryo development.

Banana embryos developing and sampling—After 3 days, the banana ECS were collected from the previous media and transferred to CS media supplemented with cefotaxime 300 mg/L. Three weeks later, the banana cells were transferred to EDM plates supplemented with cefo (cefotaxime) 300 mg/L (control), or cefo 300 mg/L and CSF 50 μg/L. The media was refreshed every 2 weeks for 8 weeks before being moved onto EDM plates supplemented with cefo 300 mg/L and CSF 25 μg/L for the final 2 weeks. Subsequently, the banana embryos were counted, sampled and genomic DNA extracted.

Embryo genotyping—Embryos were sampled from tissue culture and placed in Qiagen collection microtubes (Cat. No. 19560). DNA was extracted using an Oktapure automatic platform using LGC sbeadex plant kit.

From CSF selection, 31 embryos were recovered from EHA105 agrobacterium transformations, and 16 embryos were recovered from EHA105VirD5 agrobacterium transformations, both using a donor release strategy.

Editing in the ALS2 locus was assessed using amplicon Sanger sequencing to detect the presence of HDR donor integration using primers annealing to the genomic region around ALS2.

TABLE 12
Sequencing primers used to detect the presence of cytidine-base
editor induced mutations

| Sequencing primers: | Oligo name and sequence |
|---|---|
| ALS2 (1413 bp) | GO975 GACGATACCCCTCCCGTCTA (SEQ ID NO: 34) |
| | GO976 CAGCTCTTTCCTCCAGGTGG (SEQ ID NO: 35) |

TABLE 13
PCR conditions used for amplicon generation for Sanger sequencing

| | Temperature | Time | | Cycles |
|---|---|---|---|---|
| Initial Denaturation | 98° C. | 30 | sec | |
| Denaturation | 98° C. | 10 | sec | 35 |
| Annealing | 68° C. | 15 | sec | |
| Extension | 72° C. | 1 | min | |
| Final Extension | 72° C. | 2 | min | |

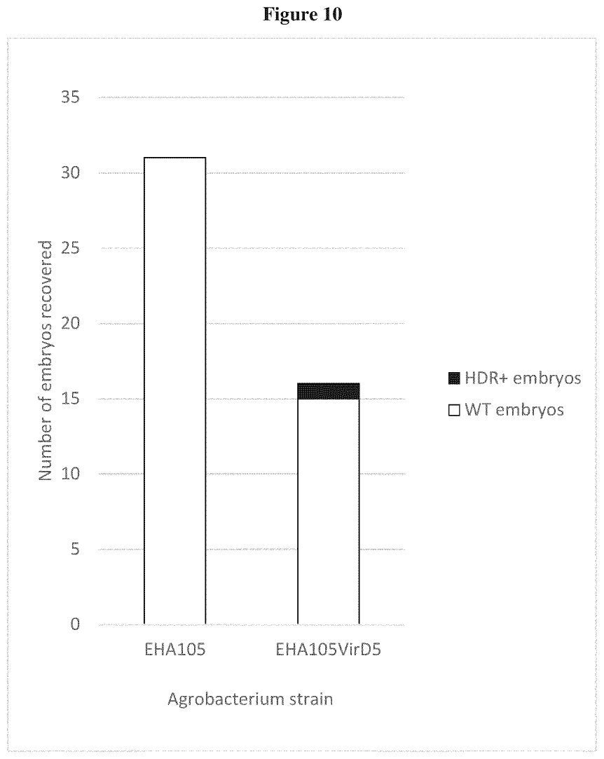

Amplicons were sent to Genewiz for Sanger sequencing. Geneious was used to assess the presence of HDR edits within the ALS2 target region in each sample. None of the 31 EHA105 embryos showed evidence of editing, however evidence of HDR (10/12 specific bases edited) was found in 1 out of 16 EHA105VirD5 embryos, as shown in FIG. 10. Due to the previous evidence for EHA105VirD5 reducing the recovery of transgenic embryos, a qPCR method was used to determine whether the embryo was transgenic.

To detect the presence of transgenes in the banana plants genomic DNA qPCR using primer sets that bind to the T-DNA was used. Primer pairs G0327, G0328 were used to detect mCherry, G0534, G0535, G1158, G1159 were used to detect nCas9 (D10A), G0989 and G0990 were used to detect promotor TaU6, and G1001 and G1002 to detect nptii. ACTIN was amplified using primers G0390 and G0391 from every sample and the amplification Ct value of this amplicon was used as a control (genomic copy number normalization).

TABLE 14
Primers used for T-DNA gDNA qPCR.

| qPCR primer | |
|---|---|
| target | Oligo name and sequence |
| ACTIN | G0390 ACCGAAGCCCCTCTTAACCC (SEQ ID NO: 16) |
| (normalisation) | G0391 GTATGGCTGACACCATCACC (SEQ ID NO: 17) |
| mCherry | G0327 AGTTCATGCGCTTCAAGGTG (SEQ ID NO: 18) |
| | G0328 ACAGCTTCAAGTAGTCGGGG (SEQ ID NO: 19) |
| nCas9 5′ | G0534 GGGCCTCACAAGAGGAGTTC (SEQ ID NO: 36) |
| | G0535 GGTCCTCCCTGTTGAGCTTC (SEQ ID NO: 37) |
| nCas9 3′ | G1158 CGTGTACGGGGACTACAAGG (SEQ ID NO: 38) |
| | G1159 CTTGTCCCACACGATCTCCC (SEQ ID NO: 39) |
| TaU6 | G0989 ACTGCCCTCTAGCTCTCACTG (SEQ ID NO: 40) |
| | G0990 CAGGCGAGGTGGGACTAAAC (SEQ ID NO: 41) |
| nptii | G0390 TTGAACGGCATGATGGCTGG (SEQ ID NO: 42) |
| | G0391 AATTCGGCTAAGCGGCTGTC (SEQ ID NO: 43) |

Reactions were all set up with 1 μl of template DNA. WT embryo DNA controls, and a water control, were used. Applied PowerUp SYBR Green master mix was used to perform PCR in the Roche LightCycler96, according to the manufacturer's recommendations.

TABLE 15
PCR conditions for T-DNA gDNA qPCR

| | Temperature | Time | | Cycles | |
|---|---|---|---|---|---|
| UDG activation | 50° C. | 2 | min | Hold | |
| Polymerase activation | 95° C. | 2 | min | Hold | |
| Denaturation | 95° C. | 15 | sec | 45 | Continuous |
| Annealing | 64° C. | 1 | Min | | Acquisition |
| High resolution melting | 95° C. | 1 | min | | |
| | 40° C. | 1 | min | | |
| | 65° C. | 1 | s | | |
| | 97° C. | 1 | s | | |

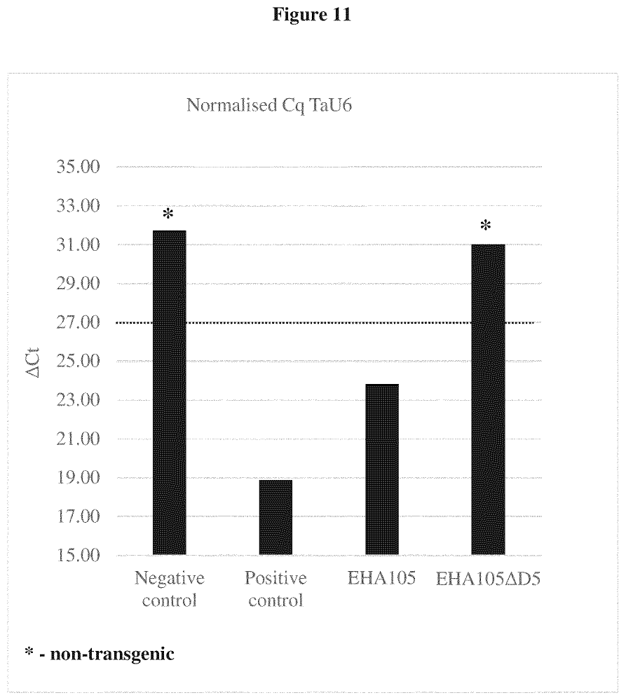

Raw data was analysed in Roche Lightcycler 96 software, and Ct values for each sample were collected, and exported to MS excel. The Ct values for the T-DNA amplicons were normalized using the ACTIN Ct levels. Once normalized, the data were plotted (FIG. 11). Primers amplifying parts of the T-DNA returned values significantly lower than ACTIN genomic control, the results show a non-transgenic, partial-HDR positive embryo using EHA105VirD5.

Further sequences

SEQ ID NO: 1
ACO1-Musa acuminata
>Ma01_g11540.1

| | |
|---|---|
| ggctatatat aagtagcaac gtggagtgac gagtgggaat agacaaagga aatggcgtgc | 60 |
| tccttcccgg tcctcgactt ggagaagctc cgtggagagg agagagagca gtccatggac | 120 |
| ctccttcgtg acgcttgcga gaaatggggc ttctttgagg tgctctcatt cttttacgtg | 180 |
| aacaagttga tgcccacgag catttgaagc taattatcat ggctttccat ttgcttgctg | 240 |
| atgtagctgc tcaaccatgg gatctcgcat gagctgatgg acgaggtgga gaggcggacc | 300 |
| aaagcgcact acgagcaatg caggaagcaa aagttcaaac agttggcgtg caaggctctc | 360 |
| aagagcggac ccgggacgga tgtcaccgac atggactggg agagcacctt cttcctgcgc | 420 |
| catctccccg tctccaacat gtccgacttc ccagacatgg acgaggagta ccggtaccgc | 480 |
| ttcgatttcc ttcgttacag cgcacccccc accaccatcg actgtagtct gccccgacta | 540 |
| accttcgcct tcaggaaggc gatgacggaa ttcgcgacgg ggttagagaa gctggcggag | 600 |
| cgtcttctcg atctgctctg cgagaacctc ggcctggagg agggttacct caagaacgcc | 660 |
| ttctacggat ccaaaggtcc gaactttggc accaaggtga gcaactaccc gccatgccct | 720 |
| cgcccggagc tgatccacgg cctgcgagcc cacaccgacg ccggcggcat catcttgctc | 780 |
| ttccaagacg accgcgtcag cggcctccag cttctcaagg atggccagtg gatcgacgtg | 840 |
| ccgcccatgc accactccat cgtggtcaac ctcggagatc agatagaggt cctctctctc | 900 |
| tctctctctc tctctctttc tctctctgcg ggacaaaaga tcaaacaaca tgagggcgtt | 960 |
| catgcaggtg atcacgaacg gcaagtacaa gagcgtgctg caccgggtgg tggctcggag | 1020 |
| cgacggcaac aggatgtcga tcgcctcctt ctacaacccg agcggcgacg ccgtcatcta | 1080 |
| ccccgcgccc tccctggtcc agaaggaagc ggaggcgtac ccgaggtttg tgttcgagga | 1140 |
| ctatatgaag ctctacgtca cgcaaaagtt tcaagcgaag cagcccaggt ttgaagcaat | 1200 |
| gaaggccacg gtaacagtca atggccaacc tacgcctaca ccttaggaca ccacgacgtc | 1260 |
| tcacgtggag atgccaccat ctattagaat gtggcatcca attgtggaaa taataagcga | 1320 |
| agcactatga acgtggcttt ttttagtctc gagggttatg tcgtcgatcc aattttccac | 1380 |
| ttct | 1384 |

SEQ ID NO: 2
ACO-Musa acuminata
>Ma07_g19730.1

| | |
|---|---|
| acactccaga tagaaagcac aagtgcaatc agggaagaaa gagcgtgtca tggattcctt | 60 |
| tccggttatc gacatggaga agcttttggg aagggagaga ggagaagcca tggagatcct | 120 |
| ccgagatgct tgcgagaaat ggggcttctt tgaggtgctg aagcatacat aactggtttt | 180 |
| gcttctttga actatatata tattgctaaa atgtactatt tgcacatgca atctgtgtgt | 240 |
| agattttaaa ccatggcatc tcacatgacc tcatggatga agtggagaag gtgaacaaag | 300 |
| accagtacaa caaatgcagg gagcaaaagt tcaacgagtt cgccaacaaa gcactggaaa | 360 |
| acgccgactc agaaatcgac cacctcgact gggaaagcac ctttttcctg cgtcatctcc | 420 |
| ccgtctccaa catttctgag atccccgatc ttgatgacca gtataggttg cacgatctga | 480 |
| tcatgatgtc atcttctggc ctggtctttt caccttgctc atcgtttcgt ttcttgggac | 540 |
| gatgactgcg tgcaggaagg cgatgaagga atttgcggca gagatggaga agctggcaga | 600 |
| gcggctgctc gacttgctgg gtgagaacct ggggctggag aaggggtacc tgaagaaagc | 660 |
| cttctctaat ggatccaagg ggccaacctt tgggaccaag gtcagcagct acccgccatg | 720 |
| cccgcgcccg gacctggtga agggcctgag ggcgcacacc gacgccggag gcatcatctt | 780 |
| gctcttccag gacgaccagg tcagcggcct gcagttcctc aaggacggcg agtggctgga | 840 |
| cgtgcccccc atgcgccacg ccatcgtcgt caacctcggc gaccagctcg aggtttgggt | 900 |
| cctctttgct ctcgtttccg ctgcccgtcg tctgtgatgt tgaatgcaac gaggtctgca | 960 |
| ggtaatcacc aatggcaagt acaagagcgt ggtgcaccgc gtggtggctc agactgatgg | 1020 |
| caacaggatg tcgattgcct ccttctacaa ccccgggagc gacgctgtga tcttcccggc | 1080 |
| ccccgctctt gtggagaagg aagcggagga gaagaaggag gtctatccga agttcgtgtt | 1140 |
| cgaggattac atgaagctct acgtcgggca taagttccag gccaaggagc caagattcga | 1200 |
| agccatgaaa gccatggaag cagttgccac ccacccaatc gctacctctt aagtgacagc | 1260 |
| ccccaagtta gtgcatgtcg ctgtacttcg cgttaggaag ctgtcgtcta tgtctatgta | 1320 |
| acccgatgga tgtgtggtat gtacgtgtgt gagccttttc taatgaagca aatcatataa | 1380 |
| tatatatata tatatatata ta | 1402 |

SEQ ID NO: 3
ACS-Musa acuminata
>Ma04_g31490.1

| | |
|---|---|
| atggggattc ccggtgacga gatcctctcc agggtcgcta cgggcgatgg ccacggtgag | 60 |
| aacacctcgt acttcgatgg ctggaaggcc tacgataatg atcctttcca cccgattcat | 120 |
| aatcccaatg gtgtcatcca aatgggactc gcagaaaacc aggtaatgct tgtttctggc | 180 |
| tctgtccatt actttctcct cctcctgctg ctgctgctgc taatgggttt cggtctgcct | 240 |
| ttcctcagct ctgcttggac ttgatgcgag attggatcag gaagaatcca caggcttcta | 300 |
| tatgcaccaa ggagggcgtt tcagagttcg aagccatcgc taacttccag gactaccatg | 360 |
| gcctgccgga cttccgtaag gtaatcaccg tctgcagcca taatgcagct cctcgatccc | 420 |
| ttactcatgc gtgccatgaa cgatgagggc acagttggat cgatatgcgt tgctatagcc | 480 |
| gaaaggtaat gacgcgatca tctatggaaa tgcacaggcc attgccaagt tcatggagaa | 540 |
| agcgagagga ggacgagcca ggttcgaccc ggagcgcata gtgatgagcg gtggagccac | 600 |
| cggagctcaa gaaacgatcg cattttgtct ggccaatccc ggggacgcct tcctcattcc | 660 |
| gacgccatac tacccagcgt acgtatgcct gttgagtcaa cattctgatc tctcaagtaa | 720 |
| ttgcgtcgtc aacttccccg ttcgaacaaa tgttccagcc gaccaatcag tcgtgcaatg | 780 |
| acccaaacga cagtcaaact tttatctgcc tgagcattga ccaaaaccac accattcaac | 840 |
| gtaattgtgg tcatgcaatc cgacactaaa gaacgacatt tggttcttct caggttcgat | 900 |
| cgagacttca ggtggagaac tggagttcag ctcctcccta ttcgctgcca cagtcacgac | 960 |
| aacttcaaga tcaccgaagc cgagcttgct gctgcctacc ggaaggcgcg cgactctaag | 1020 |
| atcagggtta aaggaatact aataaccaac ccgtcgaatc ctctgggcac aaccatggac | 1080 |
| agggagacgc taagaaccct agtaagattc gcgaacgagg aaaggatcca cctagtctgc | 1140 |
| gacgagatct tctccggcac agtcttcgac gggccggaat atgtcagtgt ggcggagata | 1200 |
| ttgcaagagg atccgtcgac ctgcgacgga gacctaatcc acatcgtcta cagcctgtcg | 1260 |
| aaggacctcg gcgtccccgg attccgtgtc ggcatcatat actcgttcaa cgacgcggtg | 1320 |
| gtcagctgcg ctcggaggat gtccagcttc ggactggtct cgacgcagac tcaacgcctg | 1380 |
| cttgcttcca tgctgggaga cgacgacttc accaccgacc tcttggcgga gagcaggagg | 1440 |
| agattaatgc acaggcacag gacgtttact gccggcctcg aaggcgtcgg cattcgttgc | 1500 |
| ttacagagca acgccggact attctgctgg atgagcttga agcctctgct gaaagacgcc | 1560 |
| acggcggagg gcgaggtcga gctgtggcgg gtgatagtga acgaggtgaa gctcaacatc | 1620 |
| tctccggggt cctcgttcca ctgcaccgag ccggggtggt tcagggcgtg ctttgccaac | 1680 |
| atggacgagg agaccatgga gacggccctg cggcggatca ggacgttcgt gcgccgggcg | 1740 |
| aacgacgcag ctactgccgc caagaccaag aagaggtggg acacatcgct tcgcctgagc | 1800 |
| ttgccacgaa ggttcgagga gatgaccgtc ctgacaccgc gtctgatgtc tcctcgctct | 1860 |
| ccgctcgttc aggccgccac ctga | 1884 |

SEQ ID NO: 4
ACS-Musa acuminata
>Ma04_g35640.1

| | |
|---|---|
| gcagcagctg cttctccttc ttctctgctc gcttcagcct tttccggtac gtacctgaga | 60 |
| taacgggtca catgaggatc tacggcgagg agcacccaaa tcagcagatc ctctctcgga | 120 |
| tcgcgaccaa cgacggccat ggcgagaact cctcctactt cgatgggtgg aaggcctacg | 180 |
| agaaggatcc tttccacctc accgacaacc ccacgggggt catccaaatg ggactcgcag | 240 |
| aaaaccaggt tagagttcct tcatggtgat gattaatcgc acatgccttc cgtcaattgc | 300 |
| cactccctgc ggttgctaat ctaatctgta tgtgggtttt gggtctttct ttcctcagct | 360 |
| ttccctcgac ttgatccgag actggatgaa gaagaacccg caggcttcga tctgcaccga | 420 |
| agaaggggtc tcagagttca aagcaattgc caactttcag gactatcatg gcctcccagc | 480 |
| cttccgaaag gtaatgattt caacccaaaa cgcagcgctg cagctgcttg tcctcactgt | 540 |
| ccaagtagct acatacgtcc aatatgataa agctgggact gacagccact tacggcccga | 600 |
| gccctgcctg ctcaccctgg ataagggata agctaatgat ggtgtgattt gctgacacgc | 660 |
| gcaggccatc gcccagttca tggagaaggt gagaggggga cgagccagat ttgacccaga | 720 |
| ccgcatcgtg atgagcggtg gagccaccgg tgctcaggaa accatcgcct tttgcctggc | 780 |
| tgatcctggc gaggccttct tgattccaac gccatattat ccggggtaag tgttcaggtg | 840 |
| tactaatcta ccgagttctt tatccggcag aggatctaat ggcatctgca tggtttccag | 900 |
| attcgatcga gacttcaggt ggaggacagg agttcagctc ctccccattc actgccacag | 960 |
| ttccaacaag ttcaagatca cccaagccgc actggagact gcttacagga aggctcgaaa | 1020 |
| ctcacacatt agagtcaaag gaatactggt gaccaaccca tcgaaccctc tgggcacaac | 1080 |
| catggacaga gagacgctga gaaccctagt cagcttcgtc aacgagaaaa ggatgcactt | 1140 |
| ggtgtgcgac gagatcttct ccggaaccgt cttcgacaag ccgagttacg tgagcgtctc | 1200 |
| cgaggtgatc gaagacgatc cctactgcga cagggatctg attcacatcg cctacagcct | 1260 |
| ctccaaggac ctgggcgtcc ctggcttccg cgtcggcgtc atatactcct acaacgacgc | 1320 |
| cgtggtcagc tgcgcgagga agatgtcgag ctttggactg gtctcgtcgc agacgcagca | 1380 |
| cctgctcgct tccatgttgg gagacgagga gttcaccacg agtttcttag cgacgagccg | 1440 |
| gacgaggttg tgcgggcggc gcagggtctt tacggacggc ctcaagcgag tcgggattca | 1500 |
| ttgcttggac ggcaacgcgg ggctgttctg ctggatggac ttgaggccgt tgctgaagga | 1560 |
| agcgacggtg gaggcggagc tccggctgtg gcgggtgatc atcaacgacg tgaagctcaa | 1620 |
| catctcgccg gggtcgtcct tccactgctc ggagccgggg tggttcaggg tatgcttcgc | 1680 |
| caacatggac gacacggcca tgaagatagc gctgaggagg atcgagagtt tcgtgtaccg | 1740 |
| ggagaacgac gccgctgtgc aggcgaagaa caagaggagg tgggacgaag cgctgcggct | 1800 |
| gagcttgcct cgtcggaggt tcgaggatcc gaccatcatg acaccacatc tgatgtctcc | 1860 |
| ccactcgcct ctcgttcaag ccgccacctg aaacatcgac agcggcgtgt ctgatgtcaa | 1920 |
| cgaaggttaa ttaccgtctg atatgttgca catttctttg ttctttggat tatttatttt | 1980 |
| tttttttttg ggaaaaatgg gttgaatgtt cccactaagt tatattagat tgttgttcgg | 2040 |
| tctcattcat gttataggaa acgaggatag aattgcttgc ctctctcttt cttttatata | 2100 |
| tggaaatatg ttacaattgg cctaagctta tttgatgaca ttaatttcac aagacaaagc | 2160 |
| cttctaatta atgtttcgga ccaaatgcag gagctcacta catacatttg ttacacttca | 2220 |
| tatgttcaaa attagtccag tttaccggtg actcagtttt aaaggttata aatggttctg | 2280 |
| attcaagtac ttatctttgg ttctgttaat tggttcaaac cgaatcgatt ttaatttaaa | 2340 |
| caatattaat ttaattaaat tttttaattg gtttaaatcg attaatcaaa tcagttgatc | 2400 |
| agggaaaata ttattgatgt cttactcaac tcgatatggt ctatactcac gtgcgtagga | 2460 |
| atgtccgaga tgtctctgag ataaaaacat cgtgctttcg tgat | 2504 |

SEQ ID NO: 5
ACS-Musa acuminata
>Ma09_g19150.1

| | |
|---|---|
| tagctcgtgt tctcccttct ccccaggctt cccagtactc gcctaagatc gtaacgtcgg | 60 |
| caatggggct ccacgttgat gaacactcaa attacaatgt cctctccagc atcgcaacga | 120 |
| gcgatggcca cggggagaac tcctcatact tcgatggctg gaaggcctac gataatgatc | 180 |
| ctttccaccc catcgacaat cctcaggggg tcatccaaat gggacttgca gaaaaccagg | 240 |
| taaatgctgt ttcacaacta gttcggtaat tatggtagtt ttttcatggc ctatggccaa | 300 |
| aaatatgcct tccgtattct cctactactt ggaatgctaa cgggtgctgc gttttcctta | 360 |
| tctcagctct gcctggactt gatgcagcag tggatcaagc agaacccaca ggcttccatt | 420 |
| tgcaccggcg agggcgtttc cgagtttaag gacgtcgcga acttccaaga ctaccacggc | 480 |
| ctgccagact tccgaaaggt aataaccatc acagtgcagc tctttagtta gtccttatca | 540 |
| tgtcataaac tgtggaccct cgagaataga ttacatcact cagataaaag atgtgcgcat | 600 |
| tatgactcac gtacatgagt ccagaacttg tatctacttg taacgacgtc aagaggattc | 660 |
| tggaaatggt gcctgctggg ctagggacaa cctcactaga ttgctttgct gtttctgaaa | 720 |
| ggctaatgat gtgatttgtg gaaacacgca ggcgattgct aggttcatgg ggaaagcgag | 780 |
| aggaggagga gctacgttcg acccggagcg cattgtaatg agcggcggag ccaccggagc | 840 |
| tcaggaaacc atcgcatttt gtctagcgaa tcctggggag gccttcctga ttccaacgcc | 900 |
| atattatcca gggtacgtag acctatccta catcaagatt ttatgtttta tgtatatttc | 960 |
| acagtgacac taatctgttt taaagaaaac tgtttgagga tgagccgatc gaactacgga | 1020 |
| ggcaacatta atataatcca gcttactggt ataaccaaaa aattagtagt caatatttgc | 1080 |
| catcgcacga ctgtgacgtc gacaagacag tctcagtata ttatatttct taattaataa | 1140 |
| cgctacacca aaaccataac cgacctaccg gccgcttgag gtttctgcac tctccggcct | 1200 |
| cattatggat ctatcggttg atatatatat atatatatat gacagcgatt tcacatttcc | 1260 |
| tgcagcttcg atcgagactt tcggtggaga actggagttc aactcctccc tattcagtgc | 1320 |
| cacagcttcg acaacttcaa gatcaccgaa cccgcgctag ttactgccta tcaaagggca | 1380 |
| caaacagcta acatcagggt taaaggaatc ctggtaacca acccttcaaa ccctctgggt | 1440 |
| acaaccttgg acagagagac actgagaacc ttagtgagct tcgccaacga gaaacggatc | 1500 |
| cacttggtgt gcgacgagat attctcgggc accgtcttcg acaagcctac ctacgtcagc | 1560 |
| gtctccgaga tcgtggaaga ggaaccatac tacgacaggg acctaattca catcgtctac | 1620 |
| agtctgtcca aggatctcgg cgtccctgga ttccgcgtgg gtgtcattta ctcgtacaat | 1680 |
| gatgcagtgg tcagctgtgc tcggaagatg tccagctttg gactggtctc gactcaaacg | 1740 |
| cagcacctac tggcttccat gctgggagat gatgacttca caaccaaatt tttggcggag | 1800 |
| agcaggagga gattgtcgcg caggcacaaa tattttactg ctggcctcca cagagttgat | 1860 |
| atcaaatgtt tggagagcaa tgcggggcta ttctgctgga tgaacttgac gcatctgcta | 1920 |
| aacgaagcca cggtggaggc ggaactcaag ctgtggcgag tgataattaa ggaggtgaag | 1980 |
| ctcaacattt caccggggtc ttcgttccac tgctctgagc cggggtggtt cagggtgtgc | 2040 |
| ttcgccaaca tggacgataa caccatggaa accgcattga agaggatcag gaagtttgtg | 2100 |
| tcccccggga atcacactgc ggctgcgcaa gccaagaaga agaacaagag gtgggacgcg | 2160 |
| gcgctccgcc taagtttgcc tcgtcggttc gaggaactga gcatcatgac acctcgcctc | 2220 |
| atgtctcctc actcgcccct tgttcaggcc gccaactgat ggtgatggat gagcgtgggc | 2280 |
| gatattaacc gacg | |

SEQ ID NO: 6
PPO2-Musa acuminata
>Ma07_g03540

| | |
|---|---|
| atggccggcc ttccttattc agctcctcac cctgccacca tctccgcttc ctccaactcc | 60 |
| tttgcatgcc ccttccgcag caaggggctt gtcttcccct accctaccag aagagcactc | 120 |
| catgttcgtc ccaacatcgc atgcaaggca ggcgaggagc acgagatcgc tgctaaggtc | 180 |
| gaccgacgcg acgtactcgt gggcctcggt gggctctgcg gagccgccgc tggccttggc | 240 |
| gggttcgata aagccgccct cgctaacccc attcaggccc ctgatctctc caagtgcggc | 300 |
| cctgccgacc tccccaccgg cgtgccagtc gtcaactgct gcccgcccta ccgtcccggt | 360 |
| gcgaagattg tggatttcaa gcggccgtcg ccgtcctccc cactccgcgt ccgccccgcc | 420 |
| gcccacttgg ttgaccccga gtacctggcc aagtacaaga aggccatcga gctcatgaag | 480 |
| gcgctcccgg ccgatgaccc tcgcaacttc atgcagcagg ccgacgtcca ctgcgcctac | 540 |
| tgcgacggcg cttacgacca gatcggcttc cccaaccttg agatccaagt ccacaacagc | 600 |
| tggctcttct tcccctggca ccgcttgtac ctctacttca acgagaggat cctcggcaag | 660 |
| ctcatcggcg acgacacctt ctcgctccct ttctggaact gggacgcacc cggcggaatg | 720 |
| atgctgcctt cgatctacgc cgatccttcg tcacccctct acgacaaact tcgcgacgcc | 780 |
| aagcaccaac ctcctgtcct tgtcgacctc gactacaatg gaaccgaccc aaccttcccc | 840 |
| gacgcccagc aaatcgatca caacctcaag atcatgtacc gccaagtctt ctccaacggc | 900 |
| aagacgccgt tgctgttctt aggctcagct taccgtgccg gtgaacagcc taaccctggc | 960 |
| gcgggctccg tcgagaacat gccgcacaac aacgtgcact tgtggaccgg cgaccgcacc | 1020 |
| cagcccaact tcgagaacat gggcaccttc tacgccgcgg cgcgcgaccc catcttcttc | 1080 |
| gcccaccacg ccaacatcga ccgcatgtgg tacctgtgga agaagctcag caggaagcac | 1140 |
| caggacttca atgactcgga ctggctcaaa gcttccttcc ttttctacga cgagaacgcc | 1200 |
| gacttagttc gggtcacggt caaggactgc ttggagaccg attggctgcg ctacacgtac | 1260 |
| caagacgtga agatcccatg ggtgaacgcc cgaccgactc ccaagctcgc caaggcgagg | 1320 |
| aaagccgcca gcagttcgct gaaagccacc gcggaggtgc agttccctgt gacgctggaa | 1380 |
| tccccggtca aagcgacggt gaagaggccc aaggtgggga ggagcggcaa ggagaaggaa | 1440 |
| gatgaggagg agatactcat agtggagggg atcgagttcg accgcgacta cttcatcaag | 1500 |
| ttcgacgtct tcgtgaacgc gacggagggc gacggcatca cggccggggc cagcgagttc | 1560 |
| gccggcagct tcgtgaacgt cccgcacaag cacaagcacc gcaaggatga gaataagctg | 1620 |
| aagacgaggc tgtgtcttgg aatcaccgac ctgctcgagg acatcggcgc ggaggacgac | 1680 |
| gacagcgtgc tcgtcaccat cgtgccgaag gcgggcaaag gaaaggtgtc cgtcggcggt | 1740 |
| cttcggattg acttttccaa g | 1761 |

1. A bacterium capable of transferring nucleotide sequences to a plant cell, comprising:
(c) a nucleotide sequence encoding vir genes, wherein the expression and/or activity of VirD5 is reduced or destroyed, and
(d) a T-DNA sequence encoding at least one site-specific DNA-editing agent operable to introduce at least one mutation in at least one target sequence in a plant cell.

2. The bacterium of claim 1, wherein the site-specific DNA editing agent comprises an endonuclease selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease.

3. The bacterium of claim 1, wherein the site-specific DNA-editing agent comprises a CRISPR-associated endonuclease or a modified CRISPR-associated endonuclease and the T-DNA sequence also encodes one or more guide RNAs specific to the at least one target sequence in a plant cell.

4. The bacterium of claim 1, wherein the site-specific DNA-editing agent comprises a CRISPR-associated endonuclease or a modified CRISPR-associated endonuclease selected from the group consisting of: a base editor, a prime editor, a Cas9 endonuclease, or an endonuclease selected from the group consisting of SpCas9, xCas9, SpCas9-NG, SaCas9, AsCpf1, LbCpf1, CjCas9, NmCas9, StCas9, TdCas9, eSpCas9, HypaCas9, Cas9-SpRY/SpG, Cas4-Cas1-Cas2 complex and MAD7.

5. The bacterium of claim 1, wherein the site-specific DNA-editing agent comprises a base editor and the T-DNA sequence also encodes one or more guide RNAs specific to the at least one target sequence in a plant cell.

6. The bacterium of claim 1, wherein the site-specific DNA-editing agent comprises a prime editor and the T-DNA sequence also encodes one or more guide RNAs that are pegRNAs specific to the at least one target sequence in a plant cell.

7. The bacterium of claim 1, wherein the site-specific DNA-editing agent comprises an endonuclease, such as a CRISPR-associated endonuclease, a modified CRISPR-associated endonuclease, a transcription activator-like effector nuclease or a zinc finger nuclease, and the T-DNA sequence also encodes at least one donor template operable to introduce the at least one mutation via homology-dependent repair (HDR) or non-homologous end-joining (NHEJ), and optionally encodes one or more guide RNAs specific to the at least one target sequence in a plant cell.

8. The bacterium of any preceding claim, wherein the bacterium is of the genus Agrobacterium, the genus Rhizobium, or the genus Ensifer, optionally wherein the bacterium is selected from the group consisting of: Agrobacterium tumefaciens, Agrobacterium fabrum str. C58, Agrobacterium genomosp, Agrobacterium sp. S2/73, Agrobacterium sp. 13-2099-1-2, Agrobacterium sp. NCPPB 925, Agrobacterium rhizogenes, Agrobacterium salinitolerans, Agrobacterium vitis, Agrobacterium arsenijevicii, Agrobacterium deltaense, Agrobacterium larrymoorei, Rhizobium sp. AB2/73, Rhizobium sp. 16-488-2b, Rhizobium sp. 16-488-2a, Rhizobium sp. 16-449-1b, Rhizobium sp. L58/93, Rhizobium sp. L245/93, Rhizobium sp. E27B/91, Rhizobium sp. K1/93, Rhizobium sp. BK007, Rhizobium tumorigenes, Rhizobium skierniewicense, Rhizobium lusitanum, Neorhizobium sp. NCHU2750, Neorhizobium galegae, Ensifer sp. YR511, and Ensifer adhaerens.

9. The bacterium of any preceding claim, wherein the expression and/or activity of VirD5 encoded by the nucleotide sequence encoding vir genes is destroyed.

10. The bacterium of any preceding claim, wherein said reduction or destruction of the expression and/or activity of VirD5 encoded by the nucleotide sequence encoding vir genesis mediated by at least one mutation in the sequence encoding VirD5.

11. The bacterium of claim 10, wherein said at least one mutation in the sequence encoding VirD5 is selected from the group consisting of:
(a) at least one nucleotide insertion;
(b) at least one nucleotide deletion;
(c) an insertion-deletion (indel);
(d) an inversion;
(e) at least one nucleotide substitution; and
(f) any combination of (a) to (e);
wherein optionally the insertion or deletion is a frame shift insertion or deletion.

12. The bacterium of any preceding claim, wherein the nucleotide sequence encoding vir genes is a plasmid, such as a Vir-helper plasmid, a Ti plasmid or a Ri plasmid.

13. The bacterium of any preceding claim, wherein the plant cell is a banana cell and at least one target sequence includes ACO or PPO, preferably ACO1 or PPO2.

14. The bacterium of any preceding claim, wherein the at least one mutation in at least one target sequence in a plant cell includes at least one mutation that results in a selectable trait in the plant cell, optionally wherein the selectable trait is herbicide resistance.

15. The bacterium of any preceding claim, wherein the at least one mutation in at least one target sequence in a plant cell includes at least one mutation in at least one acetolactate synthase (ALS) gene, wherein the at least one mutation in the ALS gene provides resistance to an ALS inhibitor, optionally wherein the ALS gene is the acetolactate synthase 1 (ALS1) gene or the acetolactate synthase 2 (ALS2) gene in banana and wherein the plant cell is a banana cell.

16. The bacterium of claim 14 or 15, wherein the T-DNA sequence encodes at least one site-specific DNA-editing agent operable to introduce at least one mutation in an additional target sequence.

17. The bacterium of any of the preceding claims, wherein the T-DNA sequence encodes:
(a) at least one CRISPR-associated endonuclease or modified CRISPR-associated endonuclease,
(b) a first guide RNA specific to a first target sequence, and
(c) a second guide RNA specific to a second target sequence,
wherein the at least one endonuclease is operable to introduce at least one mutation into the first target sequence and is operable to introduce at least one mutation into the second target sequence.

18. The bacterium of claim 17, wherein:
(A) the at least one modified CRISPR-associated endonuclease is one or more base editors operable to introduce at least one mutation into the first target sequence and operable to introduce at least one mutation into the second target sequence; or
(B) (a) the T-DNA sequence also encodes a donor template operable to introduce at least one mutation via homology-dependent repair (HDR) or non-homologous end-joining (NHEJ) into the second target sequence; or (b) the T-DNA sequence also encodes a first donor template operable to introduce at least one mutation via homology-dependent repair (HDR) or non-homologous end-joining (NHEJ) into the first target sequence and a second donor template operable to introduce at least one mutation via homology-dependent repair (HDR) or non-homologous end-joining (NHEJ) into the second target sequence; or
(C) the at least one modified CRISPR-associated endonuclease is one or more prime editors and the first guide RNA is a first pegRNA specific to a first target sequence and operable to introduce at least one mutation into the first target sequence and the second guide RNA is a second pegRNA specific to a second target sequence and operable to introduce at least one mutation into the second target sequence; or
(D) the at least one CRISPR-associated endonuclease or modified CRISPR-associated endonuclease comprise at least two different endonucleases, and wherein a first endonuclease is operable to introduce at least one mutation into the first target sequence, and a second endonuclease is operable to introduce at least one mutation into the second target sequence.

19. The bacterium of claim 17 or 18, wherein the at least one mutation introduced into the first target sequence results in a selectable trait in the plant cell, optionally wherein the selectable trait is herbicide resistance, optionally wherein the at least one mutation includes at least one mutation in at least one acetolactate synthase (ALS) gene, wherein the at least one mutation in the ALS gene provides resistance to an ALS inhibitor.

20. A method of generating a plant, plant part, plant cell or population thereof comprising at least one mutation in at least one target sequence, the method comprising contacting a plant, plant part or plant cell with the bacterium of any one of the preceding claims, optionally wherein the method further comprises regenerating said cell or plant part to obtain a whole plant.

21. The method of claim 20, wherein the at least one mutation in at least one target sequence includes:
c. at least one mutation in a first target sequence which results in a selectable trait in the plant, plant part or plant cell, optionally wherein the selectable trait is herbicide resistance, optionally wherein the first target sequence is an ALS gene and optionally wherein the mutation provides herbicide resistance to ALS inhibitors; and
d. at least one mutation in a second gene.

22. The method of claim 20, wherein at least one of the mutations in a target sequence confers a selectable trait to the plant cell, plant part or plant; optionally wherein the selectable trait is herbicide resistance; optionally wherein at least one of the target sequences is in an ALS gene and wherein the mutation provides herbicide resistance to ALS inhibitors.

23. The method of any one of claims 20-22, wherein:
(a) the genome of the plant, plant part or plant cell that is generated does not comprise any integrated T-DNA sequence; and/or
(b) the method further comprises selecting at least one plant cell, plant part or plant that comprises at least one mutation in the target sequence or sequences and does not comprise any integrated T-DNA sequence in its genome, optionally wherein said selection comprises genotyping; and/or
(c) the method further comprises selecting a cell, plant part or plant having the selectable trait,
optionally wherein the selectable trait is herbicide resistance and selecting is by selecting cells, plant parts or plants which are herbicide resistant; optionally wherein the target sequence is an ALS gene and selecting is by selecting cells, plant parts or plants which are resistant to ALS inhibitors; and/or
(d) at least 40%, optionally at least 55%, preferably at least 65% of the plant cells, plant parts or plants in the population resulting from the method do not comprise integrated T-DNA in their genome and comprise a mutation in the second gene.

24. The method of claim 20, wherein the method comprises:
d) introducing at least one mutation in a first target sequence that results in a selectable trait in the plant cell, optionally wherein the selectable trait is herbicide resistance,
e) introducing at least one mutation into a second target sequence,
f) selecting a plant, plant part, plant cell or population thereof that comprises the selectable trait, optionally by treating with a herbicide,
wherein the selected plant, plant part, plant cell or population comprises or is enriched for the mutation in the second target sequence, and optionally wherein the selected plant, plant part, plant cell or population does not comprise integrated T-DNA in their genome, or is enriched for plants, plant parts or plant cells that do not comprise integrated T-DNA in their genome.

25. The method of any one of claims 20-24, further comprising generating at least one plant embryo, plant part or plant from the cell, plant part or plant selected.

26. The method of any one of claims 20-25, wherein said plant, plant part or plant cell is banana, coffee, or rice, optionally wherein said plant, plant part or plant cell is of a banana cultivar selected from the group consisting of Musa acuminata, Musa balbisiana, Musa itinerans, autotriploid Musa acuminata ‘Cavendish’, and autotriploid Musa acuminata ‘Gros Michel’.

27. A plant, plant part, plant cell or population thereof generated by the method according to any of claims 20-26,
optionally wherein at least 40%, optionally at least 55%, preferably at least 65% of the plant cells, plant parts or plants in the population resulting from the method do not comprise integrated T-DNA in their genome and comprise a mutation in the second gene.


SEQUENCE LISTING (43 sequences)

SEQ ID NO: 1 - DNA, 1384 bp, Musa acuminata
  source 1..1384: mol_type = genomic DNA
ggctatatataagtagcaacgtggagtgacgagtgggaatagacaaaggaaatggcgtgctccttcccggtcctcgacttggagaagctccgtggagaggagagagagcagtccatggacctccttcgtgacgcttgcgagaaatggggcttctttgaggtgctctcattcttttacgtgaacaagttgatgcccacgagcatttgaagctaattatcatggctttccatttgcttgctgatgtagctgctcaaccatgggatctcgcatgagctgatggacgaggtggagaggcggaccaaagcgcactacgagcaatgcaggaagcaaaagttcaaacagttggcgtgcaaggctctcaagagcggacccgggacggatgtcaccgacatggactgggagagcaccttcttcctgcgccatctccccgtctccaacatgtccgacttcccagacatggacgaggagtaccggtaccgcttcgatttccttcgttacagcgcaccccccaccaccatcgactgtagtctgccccgactaaccttcgccttcaggaaggcgatgacggaattcgcgacggggttagagaagctggcggagcgtcttctcgatctgctctgcgagaacctcggcctggaggagggttacctcaagaacgccttctacggatccaaaggtccgaactttggcaccaaggtgagcaactacccgccatgccctcgcccggagctgatccacggcctgcgagcccacaccgacgccggcggcatcatcttgctcttccaagacgaccgcgtcagcggcctccagcttctcaaggatggccagtggatcgacgtgccgcccatgcaccactccatcgtggtcaacctcggagatcagatagaggtcctctctctctctctctctctctctctttctctctctgcgggacaaaagatcaaacaacatgagggcgttcatgcaggtgatcacgaacggcaagtacaagagcgtgctgcaccgggtggtggctcggagcgacggcaacaggatgtcgatcgcctccttctacaacccgagcggcgacgccgtcatctaccccgcgccctccctggtccagaaggaagcggaggcgtacccgaggtttgtgttcgaggactatatgaagctctacgtcacgcaaaagtttcaagcgaagcagcccaggtttgaagcaatgaaggccacggtaacagtcaatggccaacctacgcctacaccttaggacaccacgacgtctcacgtggagatgccaccatctattagaatgtggcatccaattgtggaaataataagcgaagcactatgaacgtggctttttttagtctcgagggttatgtcgtcgatccaattttccacttct

SEQ ID NO: 2 - DNA, 1402 bp, Musa acuminata
  source 1..1402: mol_type = genomic DNA
acactccagatagaaagcacaagtgcaatcagggaagaaagagcgtgtcatggattcctttccggttatcgacatggagaagcttttgggaagggagagaggagaagccatggagatcctccgagatgcttgcgagaaatggggcttctttgaggtgctgaagcatacataactggttttgcttctttgaactatatatatattgctaaaatgtactatttgcacatgcaatctgtgtgtagattttaaaccatggcatctcacatgacctcatggatgaagtggagaaggtgaacaaagaccagtacaacaaatgcagggagcaaaagttcaacgagttcgccaacaaagcactggaaaacgccgactcagaaatcgaccacctcgactgggaaagcacctttttcctgcgtcatctccccgtctccaacatttctgagatccccgatcttgatgaccagtataggttgcacgatctgatcatgatgtcatcttctggcctggtcttttcaccttgctcatcgtttcgtttcttgggacgatgactgcgtgcaggaaggcgatgaaggaatttgcggcagagatggagaagctggcagagcggctgctcgacttgctgggtgagaacctggggctggagaaggggtacctgaagaaagccttctctaatggatccaaggggccaacctttgggaccaaggtcagcagctacccgccatgcccgcgcccggacctggtgaagggcctgagggcgcacaccgacgccggaggcatcatcttgctcttccaggacgaccaggtcagcggcctgcagttcctcaaggacggcgagtggctggacgtgccccccatgcgccacgccatcgtcgtcaacctcggcgaccagctcgaggtttgggtcctctttgctctcgtttccgctgcccgtcgtctgtgatgttgaatgcaacgaggtctgcaggtaatcaccaatggcaagtacaagagcgtggtgcaccgcgtggtggctcagactgatggcaacaggatgtcgattgcctccttctacaaccccgggagcgacgctgtgatcttcccggcccccgctcttgtggagaaggaagcggaggagaagaaggaggtctatccgaagttcgtgttcgaggattacatgaagctctacgtcgggcataagttccaggccaaggagccaagattcgaagccatgaaagccatggaagcagttgccacccacccaatcgctacctcttaagtgacagcccccaagttagtgcatgtcgctgtacttcgcgttaggaagctgtcgtctatgtctatgtaacccgatggatgtgtggtatgtacgtgtgtgagccttttctaatgaagcaaatcatataatatatatatatatatatatata

SEQ ID NO: 3 - DNA, 1884 bp, Musa acuminata
  source 1..1884: mol_type = genomic DNA
atggggattcccggtgacgagatcctctccagggtcgctacgggcgatggccacggtgagaacacctcgtacttcgatggctggaaggcctacgataatgatcctttccacccgattcataatcccaatggtgtcatccaaatgggactcgcagaaaaccaggtaatgcttgtttctggctctgtccattactttctcctcctcctgctgctgctgctgctaatgggtttcggtctgcctttcctcagctctgcttggacttgatgcgagattggatcaggaagaatccacaggcttctatatgcaccaaggagggcgtttcagagttcgaagccatcgctaacttccaggactaccatggcctgccggacttccgtaaggtaatcaccgtctgcagccataatgcagctcctcgatcccttactcatgcgtgccatgaacgatgagggcacagttggatcgatatgcgttgctatagccgaaaggtaatgacgcgatcatctatggaaatgcacaggccattgccaagttcatggagaaagcgagaggaggacgagccaggttcgacccggagcgcatagtgatgagcggtggagccaccggagctcaagaaacgatcgcattttgtctggccaatcccggggacgccttcctcattccgacgccatactacccagcgtacgtatgcctgttgagtcaacattctgatctctcaagtaattgcgtcgtcaacttccccgttcgaacaaatgttccagccgaccaatcagtcgtgcaatgacccaaacgacagtcaaacttttatctgcctgagcattgaccaaaaccacaccattcaacgtaattgtggtcatgcaatccgacactaaagaacgacatttggttcttctcaggttcgatcgagacttcaggtggagaactggagttcagctcctccctattcgctgccacagtcacgacaacttcaagatcaccgaagccgagcttgctgctgcctaccggaaggcgcgcgactctaagatcagggttaaaggaatactaataaccaacccgtcgaatcctctgggcacaaccatggacagggagacgctaagaaccctagtaagattcgcgaacgaggaaaggatccacctagtctgcgacgagatcttctccggcacagtcttcgacgggccggaatatgtcagtgtggcggagatattgcaagaggatccgtcgacctgcgacggagacctaatccacatcgtctacagcctgtcgaaggacctcggcgtccccggattccgtgtcggcatcatatactcgttcaacgacgcggtggtcagctgcgctcggaggatgtccagcttcggactggtctcgacgcagactcaacgcctgcttgcttccatgctgggagacgacgacttcaccaccgacctcttggcggagagcaggaggagattaatgcacaggcacaggacgtttactgccggcctcgaaggcgtcggcattcgttgcttacagagcaacgccggactattctgctggatgagcttgaagcctctgctgaaagacgccacggcggagggcgaggtcgagctgtggcgggtgatagtgaacgaggtgaagctcaacatctctccggggtcctcgttccactgcaccgagccggggtggttcagggcgtgctttgccaacatggacgaggagaccatggagacggccctgcggcggatcaggacgttcgtgcgccgggcgaacgacgcagctactgccgccaagaccaagaagaggtgggacacatcgcttcgcctgagcttgccacgaaggttcgaggagatgaccgtcctgacaccgcgtctgatgtctcctcgctctccgctcgttcaggccgccacctga

SEQ ID NO: 4 - DNA, 2504 bp, Musa acuminata
  source 1..2504: mol_type = genomic DNA
gcagcagctgcttctccttcttctctgctcgcttcagccttttccggtacgtacctgagataacgggtcacatgaggatctacggcgaggagcacccaaatcagcagatcctctctcggatcgcgaccaacgacggccatggcgagaactcctcctacttcgatgggtggaaggcctacgagaaggatcctttccacctcaccgacaaccccacgggggtcatccaaatgggactcgcagaaaaccaggttagagttccttcatggtgatgattaatcgcacatgccttccgtcaattgccactccctgcggttgctaatctaatctgtatgtgggttttgggtctttctttcctcagctttccctcgacttgatccgagactggatgaagaagaacccgcaggcttcgatctgcaccgaagaaggggtctcagagttcaaagcaattgccaactttcaggactatcatggcctcccagccttccgaaaggtaatgatttcaacccaaaacgcagcgctgcagctgcttgtcctcactgtccaagtagctacatacgtccaatatgataaagctgggactgacagccacttacggcccgagccctgcctgctcaccctggataagggataagctaatgatggtgtgatttgctgacacgcgcaggccatcgcccagttcatggagaaggtgagagggggacgagccagatttgacccagaccgcatcgtgatgagcggtggagccaccggtgctcaggaaaccatcgccttttgcctggctgatcctggcgaggccttcttgattccaacgccatattatccggggtaagtgttcaggtgtactaatctaccgagttctttatccggcagaggatctaatggcatctgcatggtttccagattcgatcgagacttcaggtggaggacaggagttcagctcctccccattcactgccacagttccaacaagttcaagatcacccaagccgcactggagactgcttacaggaaggctcgaaactcacacattagagtcaaaggaatactggtgaccaacccatcgaaccctctgggcacaaccatggacagagagacgctgagaaccctagtcagcttcgtcaacgagaaaaggatgcacttggtgtgcgacgagatcttctccggaaccgtcttcgacaagccgagttacgtgagcgtctccgaggtgatcgaagacgatccctactgcgacagggatctgattcacatcgcctacagcctctccaaggacctgggcgtccctggcttccgcgtcggcgtcatatactcctacaacgacgccgtggtcagctgcgcgaggaagatgtcgagctttggactggtctcgtcgcagacgcagcacctgctcgcttccatgttgggagacgaggagttcaccacgagtttcttagcgacgagccggacgaggttgtgcgggcggcgcagggtctttacggacggcctcaagcgagtcgggattcattgcttggacggcaacgcggggctgttctgctggatggacttgaggccgttgctgaaggaagcgacggtggaggcggagctccggctgtggcgggtgatcatcaacgacgtgaagctcaacatctcgccggggtcgtccttccactgctcggagccggggtggttcagggtatgcttcgccaacatggacgacacggccatgaagatagcgctgaggaggatcgagagtttcgtgtaccgggagaacgacgccgctgtgcaggcgaagaacaagaggaggtgggacgaagcgctgcggctgagcttgcctcgtcggaggttcgaggatccgaccatcatgacaccacatctgatgtctccccactcgcctctcgttcaagccgccacctgaaacatcgacagcggcgtgtctgatgtcaacgaaggttaattaccgtctgatatgttgcacatttctttgttctttggattatttatttttttttttttgggaaaaatgggttgaatgttcccactaagttatattagattgttgttcggtctcattcatgttataggaaacgaggatagaattgcttgcctctctctttcttttatatatggaaatatgttacaattggcctaagcttatttgatgacattaatttcacaagacaaagccttctaattaatgtttcggaccaaatgcaggagctcactacatacatttgttacacttcatatgttcaaaattagtccagtttaccggtgactcagttttaaaggttataaatggttctgattcaagtacttatctttggttctgttaattggttcaaaccgaatcgattttaatttaaacaatattaatttaattaaattttttaattggtttaaatcgattaatcaaatcagttgatcagggaaaatattattgatgtcttactcaactcgatatggtctatactcacgtgcgtaggaatgtccgagatgtctctgagataaaaacatcgtgctttcgtgat

SEQ ID NO: 5 - DNA, 2294 bp, Musa acuminata
  source 1..2294: mol_type = genomic DNA
tagctcgtgttctcccttctccccaggcttcccagtactcgcctaagatcgtaacgtcggcaatggggctccacgttgatgaacactcaaattacaatgtcctctccagcatcgcaacgagcgatggccacggggagaactcctcatacttcgatggctggaaggcctacgataatgatcctttccaccccatcgacaatcctcagggggtcatccaaatgggacttgcagaaaaccaggtaaatgctgtttcacaactagttcggtaattatggtagttttttcatggcctatggccaaaaatatgccttccgtattctcctactacttggaatgctaacgggtgctgcgttttccttatctcagctctgcctggacttgatgcagcagtggatcaagcagaacccacaggcttccatttgcaccggcgagggcgtttccgagtttaaggacgtcgcgaacttccaagactaccacggcctgccagacttccgaaaggtaataaccatcacagtgcagctctttagttagtccttatcatgtcataaactgtggaccctcgagaatagattacatcactcagataaaagatgtgcgcattatgactcacgtacatgagtccagaacttgtatctacttgtaacgacgtcaagaggattctggaaatggtgcctgctgggctagggacaacctcactagattgctttgctgtttctgaaaggctaatgatgtgatttgtggaaacacgcaggcgattgctaggttcatggggaaagcgagaggaggaggagctacgttcgacccggagcgcattgtaatgagcggcggagccaccggagctcaggaaaccatcgcattttgtctagcgaatcctggggaggccttcctgattccaacgccatattatccagggtacgtagacctatcctacatcaagattttatgttttatgtatatttcacagtgacactaatctgttttaaagaaaactgtttgaggatgagccgatcgaactacggaggcaacattaatataatccagcttactggtataaccaaaaaattagtagtcaatatttgccatcgcacgactgtgacgtcgacaagacagtctcagtatattatatttcttaattaataacgctacaccaaaaccataaccgacctaccggccgcttgaggtttctgcactctccggcctcattatggatctatcggttgatatatatatatatatatatgacagcgatttcacatttcctgcagcttcgatcgagactttcggtggagaactggagttcaactcctccctattcagtgccacagcttcgacaacttcaagatcaccgaacccgcgctagttactgcctatcaaagggcacaaacagctaacatcagggttaaaggaatcctggtaaccaacccttcaaaccctctgggtacaaccttggacagagagacactgagaaccttagtgagcttcgccaacgagaaacggatccacttggtgtgcgacgagatattctcgggcaccgtcttcgacaagcctacctacgtcagcgtctccgagatcgtggaagaggaaccatactacgacagggacctaattcacatcgtctacagtctgtccaaggatctcggcgtccctggattccgcgtgggtgtcatttactcgtacaatgatgcagtggtcagctgtgctcggaagatgtccagctttggactggtctcgactcaaacgcagcacctactggcttccatgctgggagatgatgacttcacaaccaaatttttggcggagagcaggaggagattgtcgcgcaggcacaaatattttactgctggcctccacagagttgatatcaaatgtttggagagcaatgcggggctattctgctggatgaacttgacgcatctgctaaacgaagccacggtggaggcggaactcaagctgtggcgagtgataattaaggaggtgaagctcaacatttcaccggggtcttcgttccactgctctgagccggggtggttcagggtgtgcttcgccaacatggacgataacaccatggaaaccgcattgaagaggatcaggaagtttgtgtcccccgggaatcacactgcggctgcgcaagccaagaagaagaacaagaggtgggacgcggcgctccgcctaagtttgcctcgtcggttcgaggaactgagcatcatgacacctcgcctcatgtctcctcactcgccccttgttcaggccgccaactgatggtgatggatgagcgtgggcgatattaaccgacg

SEQ ID NO: 6 - DNA, 1761 bp, Musa acuminata
  source 1..1761: mol_type = genomic DNA
atggccggccttccttattcagctcctcaccctgccaccatctccgcttcctccaactcctttgcatgccccttccgcagcaaggggcttgtcttcccctaccctaccagaagagcactccatgttcgtcccaacatcgcatgcaaggcaggcgaggagcacgagatcgctgctaaggtcgaccgacgcgacgtactcgtgggcctcggtgggctctgcggagccgccgctggccttggcgggttcgataaagccgccctcgctaaccccattcaggcccctgatctctccaagtgcggccctgccgacctccccaccggcgtgccagtcgtcaactgctgcccgccctaccgtcccggtgcgaagattgtggatttcaagcggccgtcgccgtcctccccactccgcgtccgccccgccgcccacttggttgaccccgagtacctggccaagtacaagaaggccatcgagctcatgaaggcgctcccggccgatgaccctcgcaacttcatgcagcaggccgacgtccactgcgcctactgcgacggcgcttacgaccagatcggcttccccaaccttgagatccaagtccacaacagctggctcttcttcccctggcaccgcttgtacctctacttcaacgagaggatcctcggcaagctcatcggcgacgacaccttctcgctccctttctggaactgggacgcacccggcggaatgatgctgccttcgatctacgccgatccttcgtcacccctctacgacaaacttcgcgacgccaagcaccaacctcctgtccttgtcgacctcgactacaatggaaccgacccaaccttccccgacgcccagcaaatcgatcacaacctcaagatcatgtaccgccaagtcttctccaacggcaagacgccgttgctgttcttaggctcagcttaccgtgccggtgaacagcctaaccctggcgcgggctccgtcgagaacatgccgcacaacaacgtgcacttgtggaccggcgaccgcacccagcccaacttcgagaacatgggcaccttctacgccgcggcgcgcgaccccatcttcttcgcccaccacgccaacatcgaccgcatgtggtacctgtggaagaagctcagcaggaagcaccaggacttcaatgactcggactggctcaaagcttccttccttttctacgacgagaacgccgacttagttcgggtcacggtcaaggactgcttggagaccgattggctgcgctacacgtaccaagacgtgaagatcccatgggtgaacgcccgaccgactcccaagctcgccaaggcgaggaaagccgccagcagttcgctgaaagccaccgcggaggtgcagttccctgtgacgctggaatccccggtcaaagcgacggtgaagaggcccaaggtggggaggagcggcaaggagaaggaagatgaggaggagatactcatagtggaggggatcgagttcgaccgcgactacttcatcaagttcgacgtcttcgtgaacgcgacggagggcgacggcatcacggccggggccagcgagttcgccggcagcttcgtgaacgtcccgcacaagcacaagcaccgcaaggatgagaataagctgaagacgaggctgtgtcttggaatcaccgacctgctcgaggacatcggcgcggaggacgacgacagcgtgctcgtcaccatcgtgccgaaggcgggcaaaggaaaggtgtccgtcggcggtcttcggattgacttttccaag

SEQ ID NO: 7 - DNA, 41 bp, synthetic construct
  source 1..41: mol_type = other DNA
tcgaattcctgcagcccgggatgacaggaaagtcgaaagtt

SEQ ID NO: 8 - DNA, 31 bp, synthetic construct
  source 1..31: mol_type = other DNA
gttagcggggagggatcgatttctgccagac

SEQ ID NO: 9 - DNA, 23 bp, synthetic construct
  source 1..23: mol_type = other DNA
gatccctccccgctaacgcgggc

SEQ ID NO: 10 - DNA, 33 bp, synthetic construct
  source 1..33: mol_type = other DNA
atccaggctgcttagtgcatctaacgcttgagt

SEQ ID NO: 11 - DNA, 25 bp, synthetic construct
  source 1..25: mol_type = other DNA
cactaagcagcctggattgccgatg

SEQ ID NO: 12 - DNA, 47 bp, synthetic construct
  source 1..47: mol_type = other DNA
gaactagtggatcccccgggtcagcgtttaaacgctttgtcttgagc

SEQ ID NO: 13 - DNA, 20 bp, synthetic construct
  source 1..20: mol_type = other DNA
ttaactccacccgtgcgatc

SEQ ID NO: 14 - DNA, 20 bp, synthetic construct
  source 1..20: mol_type = other DNA
aattcgtttcaggcggcatg

SEQ ID NO: 15 - DNA, 21 bp, synthetic construct
  source 1..21: mol_type = other DNA
tgctggttggattgaaggtca

SEQ ID NO: 16 - DNA, 20 bp, synthetic construct
  source 1..20: mol_type = other DNA
accgaagcccctcttaaccc

SEQ ID NO: 17 - DNA, 20 bp, synthetic construct
  source 1..20: mol_type = other DNA
gtatggctgacaccatcacc

SEQ ID NO: 18 - DNA, 20 bp, synthetic construct
  source 1..20: mol_type = other DNA
agttcatgcgcttcaaggtg

SEQ ID NO: 19 - DNA, 20 bp, synthetic construct
  source 1..20: mol_type = other DNA
acagcttcaagtagtcgggg

SEQ ID NO: 20 - DNA, 20 bp, synthetic construct
  source 1..20: mol_type = other DNA
cctcgttgcttggggagtag

SEQ ID NO: 21 - DNA, 20 bp, synthetic construct
  source 1..20: mol_type = other DNA
gccatcaccgagttcctcag

SEQ ID NO: 22 - DNA, 20 bp, synthetic construct
  source 1..20: mol_type = other DNA
tccgcttcttctttgggctc

SEQ ID NO: 23 - DNA, 20 bp, synthetic construct
  source 1..20: mol_type = other DNA
aggaggtggaggaggtcatc

SEQ ID NO: 24 - DNA, 19 bp, synthetic construct
  source 1..19: mol_type = other DNA
tccatcaggctctcacccg

SEQ ID NO: 25 - DNA, 21 bp, synthetic construct
  source 1..21: mol_type = other DNA
ccaccaccaacatagaggact

SEQ ID NO: 26 - DNA, 20 bp, synthetic construct
  source 1..20: mol_type = other DNA
aggtgcccatgttctcgaag

SEQ ID NO: 27 - DNA, 20 bp, synthetic construct
  source 1..20: mol_type = other DNA
aagtaggtcacgtccctccc

SEQ ID NO: 28 - DNA, 21 bp, synthetic construct
  source 1..21: mol_type = other DNA
ttgatgcccacgagcatttga

SEQ ID NO: 29 - DNA, 19 bp, synthetic construct
  source 1..19: mol_type = other DNA
atccactggccatccttga

SEQ ID NO: 30 - DNA, 20 bp, synthetic construct
  source 1..20: mol_type = other DNA
gcctttcacattggaatcat

SEQ ID NO: 31 - DNA, 20 bp, synthetic construct
  source 1..20: mol_type = other DNA
atgggaagacctcgttgtca

SEQ ID NO: 32 - DNA, 20 bp, synthetic construct
  source 1..20: mol_type = other DNA
aaggtcgtccacgatacttt

SEQ ID NO: 33 - DNA, 20 bp, synthetic construct
  source 1..20: mol_type = other DNA
ggcactgctgtcaagaaatc

SEQ ID NO: 34 - DNA, 20 bp, synthetic construct
  source 1..20: mol_type = other DNA
gacgatacccctcccgtcta

SEQ ID NO: 35 - DNA, 20 bp, synthetic construct
  source 1..20: mol_type = other DNA
cagctctttcctccaggtgg

SEQ ID NO: 36 - DNA, 20 bp, synthetic construct
  source 1..20: mol_type = other DNA
gggcctcacaagaggagttc

SEQ ID NO: 37 - DNA, 20 bp, synthetic construct
  source 1..20: mol_type = other DNA
ggtcctccctgttgagcttc

SEQ ID NO: 38 - DNA, 20 bp, synthetic construct
  source 1..20: mol_type = other DNA
cgtgtacggggactacaagg

SEQ ID NO: 39 - DNA, 20 bp, synthetic construct
  source 1..20: mol_type = other DNA
cttgtcccacacgatctccc

SEQ ID NO: 40 - DNA, 21 bp, synthetic construct
  source 1..21: mol_type = other DNA
actgccctctagctctcactg

SEQ ID NO: 41 - DNA, 20 bp, synthetic construct
  source 1..20: mol_type = other DNA
caggcgaggtgggactaaac

SEQ ID NO: 42 - DNA, 20 bp, synthetic construct
  source 1..20: mol_type = other DNA
ttgaacggcatgatggctgg

SEQ ID NO: 43 - DNA, 20 bp, synthetic construct
  source 1..20: mol_type = other DNA
aattcggctaagcggctgtc